SEQUENCE LISTING (6 sequences)

SEQ ID NO: 1 - protein, 297 aa, unidentified
  source 1..297: note = sequence of Paenibacillus sp. A1
MATGFYVSGNKLYDSTGKPFVMRGVNHGHSWFKNDLNTAIPAIAKTGANTVRIVLSNGSLYTKDDLNAVKNIINVVNQNKMIAVLEVHDATGKDDYNSLDAAVNYWISIKEALIGKEDRVIVNIANEWYGTWNGSAWADGYKKAIPKLRNAGIKNTLIVDAAGWGQFPQSIVDYGQSVFAADSQKNTVFSIHMYEYAGKDAATVKANMENVLNKGLALIIGEFGGYHTNGDVDEYAIMRYGQEKGVGWLAWSWYGNSSGLNYLDMATGPNGSLTSFGNTVVNDTYGIKNTSQKAGIF

SEQ ID NO: 2 - protein, 298 aa, synthetic construct
MATGFYVSGNKLYDSTGKEFVMRGVNHGHTWFKNDLNEAIPAIAKTGANTVRIVLSNGVQYTRDDLDAVKNIINVVNQNKMIAVLEVHDATGKDDYDSLDAAINYWISIKEALIGKEDRVIVNIANEWMGTWNGSAWADGYKKAIPKLRNAGIKNTLIVDAAGWGQYPQSIVDYGQSVFAADSLKNTVFSIHMYEYAGKDAATVKANMENVLNKGLALIIGEFGCYHVNGDVDELAIMRYGQELGVGWLAWSWYGNSDGLRYLDMATGPNGSLTSFGNTVVNDTYGIKNTSQKAGIFQ

SEQ ID NO: 3 - protein, 297 aa, synthetic construct
ATGFYVSGTKLYDSTGKPFVMRGVNHSHTWFKNDLNAAIPAIAKTGANTVRIVLSNGVQYTRDDVNSVKNIISLVNQNKMIAVLEVHDATGKDDYASLDAAINYWISIKDALIGKEDRVIVNIANEWYGTWNGSAWADGYKQAIPKLRNAGIKNTLIVDAAGWGQYPQSIVDYGQSVFAADSLKNTVFSIHMYEYAGGTDAMVKANMEGVLNKGLPLIIGEFGGQHTNGDVDELAIMRYGQQKGVGWLAWSWYGNNSDLSYLDLATGPNGSLTTFGNTVVNDTNGIKATSKKAGIFQ

SEQ ID NO: 4 - protein, 298 aa, synthetic construct
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVF

SEQ ID NO: 5 - protein, 309 aa, synthetic construct
ANSGFYVSGTTLYDANGNPFVMRGINHGHAWYKDQATTAIEGIANTGANTVRIVLSDGGQWTKDDIHTVRNLISLAEDNHLVAVPEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFTGGGSDGGTSP

SEQ ID NO: 6 - protein, 309 aa, synthetic construct
ANSGFYVSGTTLYDANGNPFVMRGINHGHTWYKDQATTAIEGIANTGANTVRIVLSDGGQWTKDDIHTVRNLISLAEDNHLVAVLEVHDATGQDDIASLNRAVDYWIEMRSALIGKEKTVIINIANEWFGSWEGDPWARGYKQAIPRLRNAGLNHTLMVDAAGWGQFPQSIHDYGREVFNADPQRNTMFSIHMYEYAGGTARQVRTNIDGVLNQDLALVIGEFGHRHTNGDVDEATIMSYSEQRGVGWLAWSWKGNGPEFEYLDLSNDWAGNNLTAWGNTIVHGPYGIRETSRPVTVFTGGGSDGGTSP

(19) United States
(12) Patent Application Publication
(10) Pub. No.: US 2026/0337589 A1
(43) Pub. Date: Oct. 29, 2026

(54) MANNANASE VARIANTS AND METHODS OF USE

(71) Applicant: Danisco US Inc., Palo Alto, CA (US)

(72) Inventors: Brandon J. Burkhart, Chalfont, PA (US); Taya Feldman, Burlingame, CA (US); Thomas P. Graycar, Pacifica, CA (US); Brian James Paul, Wilmington, DE (US); Jahnavi Chandra Prasad, Melrose, MA (US); Yixin Ren, West Chester, PA (US)

(21) Appl. No.: 19/107,322

(22) PCT Filed: Aug. 29, 2023
(86) PCT No.: PCT/US2023/073055

§ 371 Date: Feb. 27, 2025

Related U.S. Application Data
(60) Provisional application No. 63/374,387, filed on Sep. 2, 2022.

(51) Int. Cl. C12N 9/24; C11D 3/386

(52) U.S. Cl. CPC C12N 9/2494; C11D 3/38636; C12Y 302/01078

(57) ABSTRACT

Disclosed herein is one or more mannanase variant, polynucleotides encoding the mannanases, compositions containing the mannanases, and methods of use thereof, including one or more mannanase variant that has improved stability compared to one or more reference mannanase. Compositions containing mannanases are suitable for use as detergents and for cleaning fabrics and hard surfaces, as well as in a variety of other industrial applications.

### CROSS-REFERENCE TO RELATED APPLICATIONS

The present application is a 371 of International Application No. PCT/US2023/073055, filed Aug. 29, 2023, which claims the benefit of U.S. Provisional Patent Application No. 63/374,387, filed Sep. 2, 2022, which are incorporated in their entirety by reference.

Disclosed herein is one or more mannanase variant, polynucleotides encoding the mannanases, compositions containing the mannanases, and methods of use thereof, including one or more mannanase variant that has improved stability compared to one or more reference mannanase. Compositions containing mannanases are suitable for use as detergents and for cleaning fabrics and hard surfaces, as well as in a variety of other industrial applications.

### REFERENCE TO SEQUENCE LISTING SUBMITTED ELECTRONICALLY

The content of the sequence listing electronically submitted with the application as an XML file (Name: 20221207_NB41943USPSP_SequenceListing; Size: 7,939 bytes; Created: Dec. 7, 2022) forms part of the application and is hereby incorporated herein by reference in its entirety.

### BACKGROUND

Mannanase enzymes, including endo-β-mannanases, have been employed in detergent cleaning compositions for the removal of gum stains by hydrolyzing mannans. A variety of mannans are found in nature, such as, for example, linear mannan, glucomannan, galactomannan, and glucogalactomannan. Each such mannan is comprised of polysaccharides that contain a β-1,4-linked backbone of mannose residues that may be substituted up to 33% with glucose residues (Yeoman et al., Adv Appl Microbiol, 70:1, 2010, Elsevier). In galactomannans or glucogalactomannnans, galactose residues are linked in alpha-1,6-linkages to the mannan backbone (Moreira and Filho, Appl Microbiol Biotechnol, 79:165, 2008). Therefore, hydrolysis of mannan to its component sugars requires endo-1,4-β-mannanases that hydrolyze the backbone linkages to generate short chain manno-oligosaccharides that are further degraded to monosaccharides by 1,4-β-mannosidases.

Although mannanases, such as, for example, endo-β-mannanases have been known in the art of industrial enzymes, there remains a need for improved mannanase variants.

### BRIEF SUMMARY

Variants, compositions and methods disclosed herein relate to a recombinant mannanase, or a recombinant polypeptide generated through conventional molecular biology techniques (see, e.g., Sambrook et al, Molecular Cloning: Cold Spring Harbor Laboratory Press).

In one embodiment, mannanase variants are provided, where the mannanase variants comprise one or more amino acid substitutions at one or more positions selected from 32, 72, 161 and 172 wherein the amino acid positions of the variant are numbered by correspondence with the amino acid sequence of SEQ ID NO: 1, and wherein the variant has at least 80% identity to the amino acid sequence of SEQ ID NO: 1.

In one embodiment, the mannanase variant is a variant comprising an amino acid substitution selected from the group consisting of 19D, 32Y, 34D, 72V, 93Q, 131S, 136P, 139R, 161G, 172F, 225N/Q, 259D, 261D/E and 276W, wherein the amino acid positions of the variant are numbered by correspondence with the amino acid sequence of SEQ ID NO: 1, and wherein the variant has at least 80% identity to the amino acid sequence of SEQ ID NO: 1.

In one embodiment, the mannanase variant is a variant comprising two or more amino acid substitutions selected from the group consisting of 19D, 32Y, 72V, 93Q, 131S, 136P, 139R, 161G, 168T, 172F, 225N/Q, 259D/E, 261D/E and 276W, wherein the amino acid positions of the variant are numbered by correspondence with the amino acid sequence of SEQ ID NO: 1, and wherein the variant has at least 80% identity to the amino acid sequence of SEQ ID NO: 1.

In one embodiment, the mannanase variant is a variant comprising amino acid substitutions selected from the group consisting of 19D-276W, 32Y-259D, 93Q-276W, 131S-276W, 136P-276W, 139R-276W, 161G-276W, 225N/Q-276W, 259D/E-276W, and 261D/E-276W.

In one embodiment, the mannanase variant is a variant comprising amino acid substitutions selected from the group consisting of 19D-131S-276W, 32Y-261D-276W, 32Y-259D-276W, 32Y-172F-259D, 168T-259D-276W, 259D-261E-276W, and 259Q-261E-276W.

In one embodiment, the mannanase variant is a variant comprising amino acid substitutions selected from the group consisting of Y061W-G259D-R261E-F276W, Y061W-T062E-G259D-R261E-F276W, Y061W-V228T-G259D-R261E-F276W, V228T-G259D-R261E-F276W, F032Y-G259D-R261E-F276W, F032Y-Y061W-Y167F-P168S-G259D-R261E-F276W, F032Y-Y061W-G259D-R261E-F276W, F032Y-Y061W-T062E-G259D-R261E-F276W, F032Y-T062E-R261D-F276W, F032Y-T062E-G259D-F276W, T062E-G259D-R261E-F276W, V059S-Q060L-G259D-R261E-F276W, V059S-Q060L-Y061W-G259D-R261E-F276W, V059S-Q060L-T062E-G259D-R261E-F276W, T062E-V228T-G259D-R261E-F276W, F032Y-T062E-G259D-R261E-F276W, F032Y-T062E-G259D-R261E-F276W-T284E, E019D-A068S-T131S-F276W, F032Y-T062E-G259D-F276W-T284E, F032Y-T062E-V228T-G259D-F276W-T284E, F032Y-T062E-I072V-G259D-F276W-T284E, F032Y-T062E-A068S-G259D-F276W-T284E, F032Y-Y061W-T062E-G259D-F276W-T284E, F032Y-V059S-Q060L-T062E-G259D-F276W-T284E, N010T-F032Y-T062E-G259D-F276W-T284E, F032Y-T062E-Y167F-P168S-V228T-G259D-F276W-T284E, F032Y-T062E-I072V-V228T-G259D-F276W-T284E, F032Y-T062E-I072V-Y167F-P168S-G259D-F276W-T284E, F032Y-T062E-A068S-V228T-G259D-F276W-T284E, F032Y-Y061W-T062E-V228T-G259D-F276W-T284E, F032Y-Y061W-T062E-Y167F-P168S-G259D-F276W-T284E, F032Y-Y061W-T062E-1072V-G259D-F276W-T284E, F032Y-Y061W-T062E-A068S-G259D-F276W-T284E, F032Y-V059S-Q060L-T062E-V228T-G259D-F276W-T284E, F032Y-V059S, Q060L-T062E-Y167F-P168S-G259D-F276W-T284E, F032Y-V059S-Q060L-T062E-I072V-G259D-F276W-T284E, F032Y-V059S-Q060L-T062E-A068S-G259D-F276W-T284E, F032Y-V059S-Q060L-Y061W-T062E-G259D-F276W-T284E, N010T-F032Y-T062E-I072V-G259D-F276W-T284E, N010T-F032Y-T062E-A068S-G259D-F276W-T284E, N010T-F032Y-Y061W-T062E-G259D-F276W-T284E, N010T-F032Y-V059S-Q060L-T062E-G259D-F276W-T284E, F032Y-T062E-I072V-Y167F-P168S-V228T-G259D-F276W-T284E, F032Y-T062E-A068S-Y167F-P168S-V228T-G259D-F276W-T284E, F032Y-T062E-A068S-I072V-Y167F-P168S-G259D-F276W-T284E, F032Y-Y061W-T062E-Y167F-P168S-V228T-G259D-F276W-T284E, F032Y-Y061W-T062E-I072V-V228T-G259D-F276W-T284E, F032Y-Y061W-T062E-1072V-Y167F-P168S-G259D-F276W-T284E, F032Y-Y061W-T062E-A068S-V228T-G259D-F276W-T284E, F032Y-Y061W-T062E-A068S-Y167F-P168S-G259D-F276W-T284E, F032Y-Y061W-T062E-A068S-I072V-G259D-F276W-T284E, F032Y-V059S-Q060L-T062E-Y167F-P168S-V228T-G259D-F276W-T284E, F032Y-V059S-Q060L-T062E-I072V-V228T-G259D-F276W-T284E, F032Y-V059S-Q060L-T062E-1072V-Y167F-P168S-G259D-F276W-T284E, F032Y-V059S-Q060L-T062E-A068S-V228T-G259D-F276W-T284E, F032Y-V059S-Q060L-T062E-A068S-Y167F-P168S-G259D-F276W-T284E, F032Y-V059S-Q060L-T062E-A068S-1072V-G259D-F276W-T284E, F032Y-V059S-Q060L-Y061W-T062E-V228T-G259D-F276W-T284E, F032Y-V059S-Q060L-Y061W-T062E-Y167F-P168S-G259D-F276W-T284E, F032Y-V059S-Q060L-Y061W-T062E-A068S-G259D-F276W-T284E, N010T-F032Y-T062E-I072V-V228T-G259D-F276W-T284E, N010T-F032Y-T062E-A068S-V228T-G259D-F276W-T284E, N010T-F032Y-T062E-A068S-I072V-G259D-F276W-T284E, N010T-F032Y-Y061W-T062E-V228T-G259D-F276W-T284E, N010T-F032Y-Y061W-T062E-Y167F-P168S-G259D-F276W-T284E, N010T-F032Y-Y061W-T062E-I072V-G259D-F276W-T284E, N010T-F032Y-V059S-Q060L-T062E-Y167F-P168S-G259D-F276W-T284E, N010T-F032Y-V059S-Q060L-T062E-I072V-G259D-F276W-T284E, F032Y-T062E-A068S-1072V-Y167F-P168S-V228T-G259D-F276W-T284E, F032Y-Y061W-T062E-1072V-Y167F-P168S-V228T-G259D-F276W-T284E, F032Y-Y061W-T062E-A068S-Y167F-P168S-V228T-G259D-F276W-T284E, F032Y-Y061W-T062E-A068S-I072V-V228T-G259D-F276W-T284E, F032Y-Y061W-T062E-A068S-I072V-Y167F-P168S-G259D-F276W-T284E, F032Y-V059S-Q060L-T062E-I072V-Y167F-P168S-V228T-G259D-F276W-T284E, F032Y-V059S-Q060L-T062E-A068S-Y167F-P168S-V228T-G259D-F276W-T284E, F032Y-V059S-Q060L-T062E-A068S-1072V-V228T-G259D-F276W-T284E, F032Y-V059S-Q060L-T062E-A068S-I072V-Y167F-P168S-G259D-F276W-T284E, F032Y-V059S-Q060L-Y061W-T062E-Y167F-P168S-V228T-G259D-F276W-T284E, F032Y-V059S-Q060L-Y061W-T062E-I072V-V228T-G259D-F276W-T284E, F032Y-V059S-Q060L-Y061W-T062E-I072V-Y167F-P168S-G259D-F276W-T284E, F032Y-V059S-Q060L-Y061W-T062E-A068S-V228T-G259D-F276W-T284E, F032Y-V059S-Q060L-Y061W-T062E-A068S-Y167F-P168S-G259D-F276W-T284E, F032Y-V059S-Q060L-Y061W-T062E-A068S-I072V-G259D-F276W-T284E, N010T-F032Y-T062E-I072V-Y167F-P168S-V228T-G259D-F276W-T284E, N010T-F032Y-T062E-A068S-Y167F-P168S-V228T-G259D-F276W-T284E, N010T-F032Y-T062E-A068S-I072V-V228T-G259D-F276W-T284E, N010T-F032Y-T062E-A068S-I072V-Y167F-P168S-G259D-F276W-T284E, N010T-F032Y-Y061W-T062E-Y167F-P168S-V228T-G259D-F276W-T284E, N010T-F032Y-Y061W-T062E-I072V-V228T-G259D-F276W-T284E, N010T-F032Y-Y061W-T062E-I072V-Y167F-P168S-G259D-F276W-T284E, N010T-F032Y-Y061W-T062E-A068S-Y167F-P168S-G259D-F276W-T284E, N010T-F032Y-Y061W-T062E-A068S-I072V-G259D-F276W-T284E, N010T-F032Y-V059S-Q060L-T062E-1072V-V228T-G259D-F276W-T284E, N010T-F032Y-V059S-Q060L-T062E-1072V-Y167F-P168S-G259D-F276W-T284E, N010T-F032Y-V059S-Q060L-T062E-A068S-V228T-G259D-F276W-T284E, N010T-F032Y-V059S-Q060L-T062E-A068S-1072V-G259D-F276W-T284E, N010T-F032Y-V059S-Q060L-Y061W-T062E-V228T-G259D-F276W-T284E, N010T-F032Y-V059S-Q060L-Y061W-T062E-Y167F-P168S-G259D-F276W-T284E, N010T-F032Y-V059S-Q060L-Y061W-T062E-A068S-G259D-F276W-T284E, F032Y-Y061W-T062E-A068S-I072V-Y167F-P168S-V228T-G259D-F276W-T284E, F032Y-V059S-Q060L-T062E-A068S-I072V-Y167F-P168S-V228T-G259D-F276W-T284E, F032Y-V059S-Q060L-Y061W-T062E-I072V-Y167F-P168S-V228T-G259D-F276W-T284E, F032Y-V059S-Q060L-Y061W-T062E-A068S-Y167F-P168S-V228T-G259D-F276W-T284E, F032Y-V059S-Q060L-Y061W-T062E-A068S-1072V-V228T-G259D-F276W-T284E, F032Y-V059S-Q060L-Y061W-T062E-A068S-1072V-Y167F-P168S-G259D-F276W-T284E, N010T-F032Y-T062E-A068S-I072V-Y167F-P168S-V228T-G259D-F276W-T284E, N010T-F032Y-Y061W-T062E-I072V-Y167F-P168S-V228T-G259D-F276W-T284E, N010T-F032Y-Y061W-T062E-A068S-Y167F-P168S-V228T-G259D-F276W-T284E, N010T-F032Y-Y061W-T062E-A068S-I072V-V228T-G259D-F276W-T284E, N010T-F032Y-V059S-Q060L-T062E-I072V-Y167F-P168S-V228T-G259D-F276W-T284E, N010T-F032Y-V059S-Q060L-T062E-A068S-Y167F-P168S-V228T-G259D-F276W-T284E, N010T-F032Y-V059S-Q060L-T062E-A068S-I072V-V228T-G259D-F276W-T284E, N010T-F032Y-V059S-Q060L-T062E-A068S-1072V-Y167F-P168S-G259D-F276W-T284E, N010T-F032Y-V059S-Q060L-Y061W-T062E-Y167F-P168S-V228T-G259D-F276W-T284E, N010T-F032Y-V059S-Q060L-Y061W-T062E-1072V-Y167F-P168S-G259D-F276W-T284E, N010T-F032Y-V059S-Q060L-Y061W-T062E-A068S-Y167F-P168S-G259D-F276W-T284E, N010T-F032Y-V059S-Q060L-Y061W-T062E-A068S-1072V-G259D-F276W-T284E, N010T-F032Y-Y061W-T062E-A068S-1072V-Y167F-P168S-V228T-G259D-F276W-T284E, N010T-F032Y-V059S-Q060L-T062E-A068S-I072V-Y167F-P168S-V228T-G259D-F276W-T284E, N010T-F032Y-V059S-Q060L-Y061W-T062E-I072V-Y167F-P168S-V228T-G259D-F276W-T284E, N010T-F032Y-V059S-Q060L-Y061W-T062E-A068S-Y167F-P168S-V228T-G259D-F276W-T284E, N010T-F032Y-V059S-Q060L-Y061W-T062E-A068S-I072V-V228T-G259D-F276W-T284E, N010T-F032Y-V059S-Q060L-Y061W-T062E-A068S-I072V-Y167F-P168S-G259D-F276W-T284E and N010T-F032Y-V059S-Q060L-Y061W-T062E-A068S-I072V-Y167F-P168S-V228T-G259D-F276W-T284E, F032Y-Y061W-T062E-G259D-R261E-G259D-R261E-F276W-T284E, F032Y-T062E-N150D-G259D-R261E-F276W-T284E, F032Y-T062E-P168S-G259D-R261E-F276W-T284E, F032Y-T062E-L235K-G259D-R261E-F276W-T284E, N010T-F032Y-Y061W-T062E-P168A-V228T-G259D-F276W-T284E, N010T-F032Y-Y061W-T062E-Y167F-P168S-V228T-G259E-F276W-T284E, F032Y-Y061W-T062E-T131S-Y167F-P168S-V228T-G259D-F276W-T284E, N010T-F032Y-Y061W-T062E-A136P-Y167F-P168S-V228T-G259D-F276W-T284E, N010T-F032Y-Y061W-T062E-K093Q-Y167F-P168S-V228T-G259D-F276W-T284E, N010T-F032Y-Y061W-T062E-Y167F-P168S-V228T-L235K-G259D-F276W-T284E, N010T-F032Y-Y061W-T062E-D139R-Y167F-P168S-V228T-G259D-F276W-T284E, N010T-F032Y-Y061W-T062E-N150D-Y167F-P168S-V228T-G259D-F276W-T284E, N010T-F032Y-Y061W-T062E-Y167F-P168A-V228T-G259D-F276W-T284E, N010T-F032Y-Y061W-T062E-T131S-Y167F-P168S-V228T-G259D-F276W-T284E, N010T-F032Y-Y061W-T062E-P168S-V228T-G259D-F276W-T284E, N010T-F032Y-Y061W-T062E-A068S-Y167F-P168A-G259D-F276W-T284E, and N010T-F032Y-Y061W-T062E-A068S-Y167F-P168A-G259E-F276W-T284E, wherein the amino acid positions of the variant are numbered by correspondence with the amino acid sequence of SEQ ID NO: 1, and wherein the variant has at least 80% identity to the amino acid sequence of SEQ ID NO: 1.

In one embodiment, the mannanase variant is a mannanase variant described herein, wherein the variant comprises an amino acid sequence having at least 85%, 90%, 91%, 92%, 93%, 94%, 95%, 96%, 97%, 98%, or 99% sequence identity to the amino acid sequence of SEQ ID NO: 1.

In one embodiment, the mannanase variant is a mannanase variant described herein, wherein said variant is derived from a parent or reference polypeptide with 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1.

In one embodiment, the mannanase variant is a mannanase variant described herein, wherein said variant has improved stability when compared to a parent or reference mannanase.

In one embodiment, the mannanase variant is a mannanase variant described herein, wherein said variant has equal or improved cleaning performance in a detergent when compared to a parent or reference mannanase.

In one embodiment, the mannanase variant is a mannanase variant described herein, wherein the mannanase variant has mannanase activity.

Also provided are polynucleotide compositions comprising a nucleic acid sequence encoding one or more mannanase variants described herein, wherein said polynucleotide is, optionally, isolated.

Further provided are enzyme compositions comprising one or more mannanase variant described herein. In one aspect, the enzyme compositions is an enzyme granule. In another aspect, the enzyme composition further comprises one or more other enzymes selected from acyl transferases, amylases, alpha-amylases, beta-amylases, alpha-galactosidases, arabinases, arabinosidases, aryl esterases, beta-galactosidases, beta-glucanases, carrageenases, catalases, cellulases, chondroitinases, cutinases, dispersins, endo-glucanases, endo-beta-mannanases, exo-beta-mannanases, esterases, exo-mannanases, galactanases, glucoamylases, hemicellulases, hexosaminidase, hyaluronidases, keratinases, laccases, lactases, ligninases, lipases, lipolytic enzymes, lipoxygenases, lysozyme, mannanases, metalloproteases, nucleases, oxidases, oxidoreductases, pectate lyases, pectin acetyl esterases, pectinases, pentosanases, perhydrolases, peroxidases, PETases, phenoloxidases, phosphatases, phospholipases, phytases, polyesterases, polygalacturonases, proteases, pullulanases, reductases, rhamnogalacturonases, subtilisins, tannases, transglutaminases, xylan acetyl-esterases, xylanases, and xylosidases; and combinations thereof.

Further provided are cleaning compositions comprising one or more mannanase variant described herein. In one aspect, the cleaning composition is a detergent composition selected from the group consisting of a laundry detergent, a fabric softening detergent, a dishwashing detergent, a medical instrument cleaning detergent, and a hard-surface cleaning detergent.

The disclosure also provides methods of cleaning. In one embodiment, the method is a method of cleaning comprising, contacting a surface or an item in need of cleaning with an effective amount of a mannanase variant described herein or the enzyme composition described herein; and optionally further comprising the step of rinsing said surface or item after contacting said surface or item with said variant or enzyme composition. In one aspect the item is dishware or fabric.

In some embodiments, the mannanase variant has cleaning activity in a detergent composition. In some embodiments, the mannanase variant has mannanase activity in the presence of a protease. In some embodiments, the mannanase variant retains at least 50% mannanase activity in the presence of a protease. In some embodiments, the mannanase variant is capable of hydrolyzing a substrate selected from the group consisting of guar gum, locust bean gum, and combinations thereof. In some embodiments, the mannanase variant does not further comprise a carbohydrate-binding module.

In some embodiments, the method is a method of cleaning comprising, contacting a surface or an item in need of cleaning with an effective amount of a mannanase described herein or the enzyme composition comprising a mannanase variant described herein; and optionally further comprising the step of rinsing said surface or item after contacting said surface or item with said variant or enzyme composition.

Yet further embodiments are directed to a method of cleaning comprising contacting a surface or item comprising a soil or stain comprising mannan with a (i) mannanase variant or recombinant polypeptide, or (ii) a cleaning composition described herein, wherein the mannan contained is said soil or stain is hydrolyzed.

Some embodiments are further directed to nucleic acids or isolated nucleic acids encoding the mannanase variants or recombinant polypeptides described herein. Further embodiments are directed to an expression vector comprising a nucleic acid or isolated nucleic acid described herein operably linked to a regulatory sequence. Even further embodiments are directed to a host cell comprising an expression vector described herein, or nucleic acids encoding the mannanase variants or recombinant polypeptides described herein. In some embodiments, the host cell is a bacterial cell or a fungal cell. Still further embodiments are directed to methods of producing a mannanase variant described herein comprising: stably transforming a host cell with an expression vector comprising a polynucleotide encoding the mannanase variant; culturing the transformed host cell under suitable conditions to produce the mannanase variant; and recovering the mannanase variant.

### DETAILED DESCRIPTION

In one embodiment, the present disclosure provides one or more mannanase variant comprising one or more amino acid substitutions as described in more detail below. In some embodiments, the variants provided herein demonstrate one or more improved properties, such as an improved stability when compared to a reference mannanase having the amino acid sequence of SEQ ID NO: 1 or 2. The mannanase variants provided herein find use in the preparation of cleaning compositions (e.g. automatic dishwashing compositions). In addition, the mannanase variants provided herein also find use in methods of cleaning (e.g. dish washing methods) using such variants or compositions comprising such mannanase variants.

Unless otherwise indicated herein, one or more mannanase variant described herein can be made and used by a variety of techniques used in molecular biology, microbiology, protein purification, protein engineering, protein and DNA sequencing, recombinant DNA fields, and industrial enzyme use and development.

In one embodiment, the one or more mannanase variant described herein have glycosyl hydrolase activity and/or are stable in the presence of a protease. These features of the mannanase variants described herein make them well suited for use in a variety of cleaning and other industrial applications, for example, where the enzyme can hydrolyze mannans in the presence of surfactant, protease, and/or other components found in a detergent composition.

The following terms are defined for clarity. Terms and abbreviations not defined should be accorded their ordinary meaning as used in the art. For example, technical and scientific terms not defined herein have the same meaning as commonly understood by one of ordinary skill in the art to which this disclosure pertains (See, e.g., Singleton and Sainsbury, Dictionary of Microbiology and Molecular Biology, 2d Ed., John Wiley and Sons, NY 1994; and Hale and Marham, The Harper Collins Dictionary of Biology, Harper Perennial, NY 1991). As used herein, the singular terms “a,” “an,” and “the” include the plural reference unless the context clearly indicates otherwise. Unless otherwise indicated, nucleic acid sequences are written left to right in 5′ to 3′ orientation; and amino acid sequences are written left to right in amino to carboxy orientation. Each numerical range used herein includes every narrower numerical range that falls within such broader numerical range, as if such narrower numerical ranges were all expressly written herein.

As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

The nomenclature of the amino acid substitutions of the one or more mannanase variants described herein uses one or more of the following: position; position:amino acid substitution(s); or starting amino acid(s):position:amino acid substitution(s). Reference to a “position” (e.g. 5, 8, 17, 22, etc) encompasses any starting amino acid that may be present at such position, and any substitution that may be present at such position. Reference to a “position: amino acid substitution(s)” (e.g. 1S/T/G, 3G, 17T, etc) encompasses any starting amino acid that may be present at such position and the one or more amino acid(s) with which such starting amino acid may be substituted. Reference to a position can be recited in several forms, for example, position 003 can also be referred to as position 03 or 3. Reference to a starting or substituted amino acid may be further expressed as several starting, or substituted amino acids separated by a foreslash (“/”). For example, D275S/K indicates position 275 is substituted with serine(S) or lysine (K) and P/S197K indicates that starting amino acid proline (P) or serine(S) at position 197 is substituted with lysine (K). Reference to an X as the amino acid in a position, refers to any amino acid at the recited position.

The position of an amino acid residue in a given amino acid sequence is numbered by correspondence with the amino acid sequence of SEQ ID NO:1. That is, the amino acid sequence of SEQ ID NO: 1 serves as a reference sequence for numbering of positions of an amino acid residue. For example, the amino acid sequence of one or more mannanase variant described herein is aligned with the amino acid sequence of SEQ ID NO: 1 using an alignment algorithm as described herein, and each amino acid residue in the given amino acid sequence that aligns (preferably optimally aligns) with an amino acid residue in SEQ ID NO:1 is conveniently numbered by reference to the numerical position of that corresponding amino acid residue. Sequence alignment algorithms, such as, for example, described herein will identify the location or locations where insertions or deletions occur in a subject sequence when compared to a query sequence (also sometimes referred to as a “reference sequence”).

The terms “mannan endo-1,4-β-mannosidase,” “endo-1,4-β-mannanase,” “endo-β-1,4-mannase,” “B-mannanase B,” “β-1, 4-mannan 4-mannanohydrolase,” “endo-β-mannanase,” “β-D-mannanase,” “1,4-β-D-mannan mannanohydrolase,” or “endo-β-mannanase” (EC 3.2.1.78) refer to an enzyme capable of the random hydrolysis of 1,4-β-D-mannosidic linkages in mannans, galactomannans and glucomannans. Endo-1,4-β-mannanases are members of several families of glycosyl hydrolases, including GH26 and GH5. In particular, endo-β-mannanases constitute a group of polysaccharases that degrade mannans and denote enzymes that are capable of cleaving polyose chains containing mannose units (i.e., are capable of cleaving glycosidic bonds in mannans, glucomannans, galactomannans and galactogluco-mannans). The “endo-β-mannanases” described herein may possess additional enzymatic activities (e.g., endo-1,4-β-glucanase, 1,4-β-mannosidase, and cellodextrinase activities).

The terms “mannanase,” “mannosidic enzyme,” “mannolytic enzyme,” “mannanase enzyme,” “mannanase polypeptides,” or “mannanase proteins” refer to an enzyme, polypeptide, or protein that can degrade mannan. The mannanase enzyme may, for example, be an endo-β-mannanase, an exo-β-mannanase, or a glycosyl hydrolase. As used herein, mannanase activity may be determined according to any procedure known in the art (See, e.g., Lever, Anal. Biochem, 47:273, 1972; Eriksson and Winell, Acta Chem. Scand., (1968), 22:1924; U.S. Pat. No. 6,602,842; and WO 95/35362A1).

As used herein, “mannans” are polysaccharides having a backbone composed of β-1,4-linked mannose; “glucomannans” are polysaccharides having a backbone of more or less regularly alternating β-1,4 linked mannose and glucose; “galactomannans” and “galactoglucomannans” are mannans and glucomannans with alpha-1,6 linked galactose side-branches. These compounds may be acetylated. The degradation of galactomannans and galactoglucomannans is facilitated by full or partial removal of the galactose side-branches. Further, the degradation of the acetylated mannans, glucomannans, galactomannans and galactoglucomannans is facilitated by full or partial deacetylation. Acetyl groups can be removed by alkali or by mannan acetylesterases. The oligomers that are released from the mannanases or by a combination of mannanases and alpha-galactosidase and/or mannan acetyl esterases can be further degraded to release free maltose by β-mannosidase and/or β-glucosidase.

The terms “protease” and “proteinase” refer to an enzyme that has the ability to break down proteins and peptides. A protease has the ability to conduct “proteolysis,” by hydrolysis of peptide bonds that link amino acids together in a peptide or polypeptide chain forming the protein. This activity of a protease as a protein-digesting enzyme is referred to as “proteolytic activity.” Many well-known procedures exist for measuring proteolytic activity. For example, proteolytic activity may be ascertained by comparative assays that analyze the respective protease's ability to hydrolyze a suitable substrate. Exemplary substrates useful in the analysis of protease or proteolytic activity, include, but are not limited to, di-methyl casein (Sigma C-9801), bovine collagen (Sigma C-9879), bovine elastin (Sigma E-1625), and Keratin Azure (Sigma-Aldrich K8500). Colorimetric assays utilizing these substrates are well known in the art (See e.g., WO99/34011 and U.S. Pat. No. 6,376,450).

The term “modification” refers to any change or alteration in an amino acid sequence, including the substitution of an amino acid at the identified position of the amino acid sequence of interest with an amino acid that is different from the starting amino acid, deletion of an amino acid at the identified position of the amino acid sequence of interest, insertion of an amino acid at the identified position of the amino acid sequence of interest, replacement of an amino acid side chain in the amino acid sequence of interest, and or chemical modification of the amino acid sequence of interest.

The terms “catalytic activity” or “activity” describes quantitatively the conversion of a given substrate under defined reaction conditions. The term “residual activity” is defined as the ratio of the catalytic activity of the enzyme under a certain set of conditions to the catalytic activity under a different set of conditions. The term “specific activity” describes quantitatively the catalytic activity per amount of enzyme under defined reaction conditions.

The term “pH-stability” describes the ability of a protein to withstand a limited exposure to pH-values significantly deviating from the pH where its stability is optimal (e.g., more than one pH-unit above or below the pH-optimum), without losing its activity under conditions where its activity is measurable.

The term “detergent stability” refers to the stability of a specified detergent composition component (such as a hydrolytic enzyme) in a detergent composition mixture.

The term “perhydrolase” refers to an enzyme capable of catalyzing a reaction that results in the formation of a peracid suitable for applications such as cleaning, bleaching, and disinfecting.

The term “aqueous,” as used in the phrases “aqueous composition” and “aqueous environment” refers to a composition that is made up of at least 50% water. An aqueous composition may contain at least 50%, 60%, 70%, 80%, 90%, 95%, 97%, 98%, or 99% water.

The term “surfactant” refers to any compound generally recognized in the art as having surface active qualities. Surfactants generally include anionic, cationic, nonionic, and zwitterionic compounds, which are further described, herein.

The term “surface property” is used in reference to electrostatic charge, as well as properties such as the hydrophobicity and hydrophilicity exhibited by the surface of a protein.

The term “chelator stability” refers to mannanase variants of the present disclosure that retain a specified amount of enzymatic activity over a given period of time under conditions prevailing during the mannosidic, hydrolyzing, cleaning, or other process disclosed herein, for example while exposed to or contacted with chelating agents. In some embodiments, the mannanase variant retains at least about 50%, about 60%, about 70%, about 75%, about 80%, about 85%, about 90%, about 92%, about 95%, about 96%, about 97%, about 98%, or about 99% mannanase activity after contact with a chelating agent over a given time period, for example, at least about 10 minutes, about 20 minutes, about 40 minutes, about 60 minutes, about 100 minutes, etc.

The terms “thermal stability” and “thermostable” refer to mannanase variants that retain a specified amount of enzymatic activity after exposure to elevated temperatures over a given period of time under conditions prevailing during the mannosidic, hydrolyzing, cleaning, or other process, for example, while exposed to elevated temperatures. In some embodiments, the mannanase retains at least about 50%, about 60%, about 70%, about 75%, about 80%, about 85%, about 90%, about 92%, about 95%, about 96%, about 97%, about 98%, or about 99% mannanase activity after exposure to elevated temperatures, for example, at least about 50 °C, about 55 °C, about 60 °C, about 65 °C, or about 70 °C, over a given time period, for example, at least about 5 minutes, 10 minutes, 15 minutes, 20 minutes, 30 minutes, 40 minutes, 50 minutes, 60 minutes, 120 minutes, 180 minutes, 240 minutes, 300 minutes, etc.

The term “cleaning activity” refers to the cleaning performance achieved by a mannanase variant under conditions prevailing during the mannosidic, hydrolyzing, cleaning, or other process disclosed herein. In some embodiments, cleaning performance is determined by the application of various cleaning assays concerning enzyme sensitive stains arising from food products, household agents or personal care products. Some of these stains include, for example, ice cream, ketchup, BBQ sauce, mayonnaise, soups, chocolate milk, chocolate pudding, frozen desserts, shampoo, body lotion, sun protection products, toothpaste, locust bean gum, or guar gum as determined by various chromatographic, spectrophotometric or other quantitative methodologies after subjection of the stains to standard wash conditions. Exemplary assays include, but are not limited to those described in WO99/34011, U.S. Pat. Nos. 6,605,458, and 6,566,114, as well as those methods described in the Examples.

The terms “clean surface” and “clean textile” refer to a surface or textile respectively that has a percent stain removal of at least 10%, preferably at least 15%, 20%, 25%, 30%, 35%, or 40% of a soiled surface or textile.

The term “effective amount” when used in conjunction with a mannanase variant refers to the quantity of mannanase variant needed to achieve the desired level of enzymatic activity in the specified cleaning composition. Such effective amounts are readily ascertained by one of ordinary skill in the art and are based on many factors, such as the particular mannanase variant that is used, the cleaning application, the specific composition of the cleaning composition, and whether a liquid or dry (e.g., granular, bar, powder, solid, liquid, tablet, gel, paste, foam, sheet, or unit dose) composition is required.

The term “adjunct ingredient” when used in conjunction with a cleaning composition means any liquid, solid or gaseous material selected for the particular type of cleaning composition desired and the form of the product (e.g., liquid, granule, powder, bar, paste, spray, tablet, gel, unit dose, sheet, or foam composition), which materials are also preferably compatible with the mannanase variant used in the composition. In some embodiments, granular compositions are in “compact” form, while in other embodiments, the liquid compositions are in a “concentrated” form.

The terms “cleaning compositions” and “cleaning formulations” refer to admixtures of chemical ingredients that find use in the removal of undesired compounds (e.g., soil or stains) from items or surfaces to be cleaned, such as, for example, fabric, dishes, contact lenses, solid surfaces, hair, skin, and teeth. The compositions or formulations may be in the form of a liquid, gel, granule, powder, bar, paste, spray tablet, gel, unit dose, sheet, or foam, depending on the surface or item to be cleaned and the desired form of the composition or formulation.

The terms “detergent composition” and “detergent formulation” refer to mixtures of chemical and/or biological ingredients intended for use in a wash medium for the cleaning of soiled objects. Detergent compositions/formulations generally include at least one surfactant, and may optionally include hydrolytic enzymes, oxido-reductases, builders, bleaching agents, bleach activators, bluing agents, fluorescent dyes, caking inhibitors, masking agents, enzyme activators, antioxidants, solubilizers, and one or more microorganisms or microbes or microbial extracts. Microorganisms may be used as the only biologically active ingredient, but they may also be used in conjunction with one or more of the enzymes described herein. A bacillus strain having the deposit accession number PTA-7543, for example, may be used to reduce malodor as described in WO 2012/112718. Other purposes could include in-situ production of desirable biological compounds, or inoculation/population of a locus with the microorganism(s) to competitively prevent other non-desirable microorganisms form populating the same locus (competitive exclusion).

The term “dishwashing composition” refers to all forms of compositions including, for example, granular, unit-dose, and liquid forms for cleaning dishware and cutlery. In some embodiments, the dishwashing composition is an “automatic dishwashing” composition that finds use in automatic dishwashing machines. The term “dishware” refers to dishes (e.g., plates, cups, glasses, bowls, and containers) and cutlery (e.g., utensils including, but not limited to spoons, knives, and forks) of any material, including but not limited to ceramics, plastics, metals, china, glass, and acrylics.

The term “bleaching” refers to the treatment of a material (e.g., fabric, laundry, pulp, etc.) or surface for a sufficient length of time and under appropriate pH and temperature conditions to effect a brightening (i.e., whitening) and/or cleaning of the material. Examples of chemicals suitable for bleaching include but are not limited to ClO₂, H₂O₂, peracids, and NO₂.

The term “wash performance” of a mannanase variant refers to the contribution of the variant to washing that provides additional cleaning performance to the detergent composition. Wash performance is compared under relevant washing conditions. The term “relevant washing conditions” is used herein to indicate the conditions, particularly washing temperature, time, washing mechanics, suds concentration, type of detergent, and water hardness, actually used in households in a dish or laundry detergent market segment.

As used herein, the term “disinfecting” refers to the removal of contaminants from the surfaces, as well as the inhibition or killing of microbes on the surfaces of items.

The “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically about 17 to about 35% by weight of the total composition. In contrast, in compact compositions, the filler salt is present in amounts not exceeding about 15% of the total composition. In some embodiments, the filler salt is present in amounts that do not exceed about 10%, or more preferably, about 5%, by weight of the composition. In some embodiments, the inorganic filler salts are selected from the alkali and alkaline-earth-metal salts of sulfates and chlorides. In some embodiments, a preferred filler salt is sodium sulfate.

The term “fabric” refers to, for example, woven, knit, and non-woven material, as well as staple fibers and filaments that can be converted to, for example, yarns and woven, knit, and non-woven fabrics. The term encompasses material made from natural, as well as synthetic (e.g., manufactured) fibers.

A nucleic acid or polynucleotide is “isolated” when it is at least partially or completely separated from other components, including but not limited to, for example, other proteins, nucleic acids, and cells. Similarly, a polypeptide, protein or peptide is “isolated” when it is at least partially or completely separated from other components, including but not limited to, for example, other proteins, nucleic acids, and cells. On a molar basis, an isolated species is more abundant than are other species in a composition. For example, an isolated species may comprise at least about 60%, 65%, 70%, 75%, 80%, 85%, 90%, 91%, 92%, 93%, 94%, 95%, 96%, 97%, 98%, 99%, or 100% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a nucleic acid or a protein sample, respectively, followed by visualization upon staining. If desired, a high-resolution technique, such as high performance liquid chromatography (HPLC) or a similar means can be utilized for purification of the material.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, 65%, 70%, 75%, 80%, 85%, 90%, 91%, 92%, 93%, 94%, 95%, 96%, 97%, 98%, 99%, 99.5%, 99.6%, 99.7%, 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than in a starting composition.

As used herein, a “polypeptide” refers to a molecule comprising a plurality of amino acids linked through peptide bonds. The terms “polypeptide,” “peptide,” and “protein” are used interchangeably. Proteins may optionally be modified (e.g., glycosylated, phosphorylated, acylated, farnesylated, prenylated, and sulfonated) to add functionality. Where such amino acid sequences exhibit activity, they may be referred to as an “enzyme”. The conventional one-letter or three-letter codes for amino acid residues are used, with amino acid sequences being presented in the standard amino-to-carboxy terminal orientation (i.e., N→C).

The terms “polynucleotide” encompasses DNA, RNA, heteroduplexes, and synthetic molecules capable of encoding a polypeptide. Nucleic acids may be single-stranded or double-stranded, and may have chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence. Unless otherwise indicated, nucleic acid sequences are presented in a 5′-to-3′ orientation.

As used herein, the terms “wild-type” and “native” refer to polypeptides or polynucleotides that are found in nature.

The terms “wild-type” and “parental”, with respect to a polypeptide, refer to a naturally-occurring polypeptide that does not include a man-made substitution, insertion, or deletion at one or more amino acid positions. Similarly, the terms “wild-type” and “parental”, with respect to a polynucleotide, refer to a naturally-occurring polynucleotide that does not include a man-made substitution, insertion, or deletion at one or more nucleosides. However, note that a polynucleotide encoding a wild-type or parental polypeptide is not limited to a naturally-occurring polynucleotide, and encompasses any polynucleotide encoding the wild-type or parental polypeptide.

The term “reference”, with respect to a polypeptide, refers to a naturally-occurring polypeptide that does not include a man-made substitution, insertion, or deletion at one or more amino acid positions, as well as a polypeptide that includes one or more man-made substitutions, insertions, or deletions at one or more amino acid positions. Similarly, the term “reference”, with respect to a polynucleotide, refers to a naturally-occurring polynucleotide that does not include a man-made substitution, insertion, or deletion of one or more nucleosides, as well as a polynucleotide that includes one or more man-made substitutions, insertions, or deletions at one or more nucleosides. However, note that a polynucleotide encoding a wild-type or parental polypeptide is not limited to a naturally-occurring polynucleotide, and encompasses any polynucleotide encoding the wild-type or parental polypeptide.

The one letter code “Z” identifies an insertion or deletion in a parent or reference amino acid sequence. For an insertion relative to a parent or reference sequence, the one letter code “Z” is on the left side of the position number and further includes a number (e.g., 0.01) before each amino acid being inserted therein to indicate the order of the insertions. For example, the insertion of one amino acid, glutamine (Q), at position 298 would be depicted as “Z298.01Q”; the insertion of one amino acid, X (where X can be any amino acid) at position 298 would be depicted as “Z298.01X”; and the insertion of three amino acids alanine (A), serine(S) and tyrosine (Y) between position 87 and 88 would be depicted as “Z87.01A/Z87.02S/Z87.03Y”. For a deletion, the one letter code “Z” is on the right side of the position number. For example, the deletion of an alanine (A) from position 100 would be depicted as A100Z. A combination of some of the above insertions and deletions would be depicted as: “G87S/Z87.01A/Z87.02S/Z87.03Y/A100Z”.

The term “mannanase variant” refers to a polypeptide that is derived from a reference polypeptide by the substitution, addition, or deletion, of one or more amino acids, typically by recombinant DNA techniques. A mannanase variant may differ from a reference polypeptide by a small number of amino acid residues and may be defined by the level of primary amino acid sequence homology/identity with the reference polypeptide over the length of the catalytic domain. For example, a mannanase variant has at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity with a reference polypeptide. The reference polypeptide includes, but is not limited to, naturally occurring and recombinant mannanases, such as but not limiting to mannanases within the GH5_8 sub family of mannanases (endo-1,4 β-mannosidases, EC 3.2.1.78). (Aspeborg et al (2012), “Evolution, substrate specificity and subfamily classification of glycosyl hydrolase family 5 (GH5)”, BMC Evolutionary Biology, 12:186.) Exemplary reference GH5_8 bacterial mannanases include, for example, NDL-Clade mannanases, such as, for example, PspMan4 (SEQ ID NO:1), PspMan138 (SEQ ID NO:2) and PspMan9 (SEQ ID NO:3); and other mannanases such as, for example, SEQ ID NOs: 4-6

One or more amino acid substitutions of the mannanase variants described herein can be included in any known mannanase, including but not limited to the mannanase of SEQ ID NO: 1, SEQ ID NO: 2, SEQ ID NO: 3, SEQ ID NO: 4, SEQ ID NO: 5 or SEQ ID NO: 6.

The term “variant polynucleotide” refers to a polynucleotide that encodes a mannanase variant, has a specified degree of homology/identity with a parent polynucleotide, or hybridizes under stringent conditions to a parent polynucleotide or the complement thereof. For example, a variant polynucleotide has at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% nucleotide sequence identity with a parent polynucleotide.

Sequence identity may be determined from protein sequence alignments using known programs such as BLAST, ALIGN, and CLUSTAL using standard parameters. (See, e.g., Altschul et al. [1990] J. Mol. Biol. 215:403-410; Henikoff et al. [1989] Proc. Natl. Acad. Sci. USA 89:10915; Karin et al. [1993] Proc. Natl. Acad. Sci. USA 90:5873; and Higgins et al. [1988] Gene 73:237-244). Software for performing BLAST analyses is publicly available through the National Center for Biotechnology Information (NCBI). Databases may also be searched using FASTA (Pearson et al. [1988] Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another useful algorithm for alignment and comparison of multiple protein sequences is the MUSCLE program (Robert C. Edgar. MUSCLE: multiple sequence alignment with high accuracy and high throughput Nucl. Acids Res. (2004) 32 (5): 1792-1797) available from Geneious software (Biomatters Ltd.).

The term “derived from” encompasses the terms “originated from,” “obtained from,” “obtainable from,” “isolated from,” and “created from” and generally indicates that one specified material find its origin in another specified material or has features that can be described with reference to the another specified material.

The term “hybridization” refers to the process by which a strand of nucleic acid joins with a complementary strand through base pairing, as known in the art.

The term “hybridization conditions” refers to the conditions under which hybridization reactions are conducted. These conditions are typically classified by degree of “stringency” of the conditions under which hybridization is measured. The degree of stringency can be based, for example, on the melting temperature (Tₘ) of the nucleic acid binding complex or probe. For example, “maximum stringency” typically occurs at about Tₘ-5 °C (5 °C below the Tₘ of the probe); “high stringency” at about 5-10 °C below the Tₘ; “intermediate stringency” at about 10-20 °C below the Tₘ of the probe; and “low stringency” at about 20-25 °C below the Tₘ. Alternatively, or in addition, hybridization conditions can be based upon the salt or ionic strength conditions of hybridization and/or one or more stringency washes, e.g., 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used).

The terms “substantially similar” and “substantially identical” in the context of at least two nucleic acids or polypeptides means that a polynucleotide or polypeptide comprises either a sequence that has at least about 90%, 91%, 92%, 93%, 94%, 95%, 96%, 97%, 98%, or 99% identity to a parent or reference sequence, or a sequence that includes amino acid substitutions, insertions, deletions, or modifications made only to circumvent the present description without adding functionality.

The term “expression vector” refers to a DNA construct containing a DNA sequence that encodes the specified polypeptide and is operably linked to a suitable control sequence capable of effecting the expression of the polypeptides in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself.

The term “recombinant” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally, recombinant nucleic acids, polypeptides, and cells based thereon, have been manipulated by man such that they are not identical to related nucleic acids, polypeptides, and cells found in nature.

The term “signal sequence” refers to a sequence of amino acids bound to the N-terminal portion of a polypeptide, and which facilitates the secretion of the mature form of the protein from the cell. The mature form of the extracellular protein lacks the signal sequence which is cleaved off during the secretion process.

The terms “selective marker” or “selectable marker” refer to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell. The term “selectable gene product” refers to a gene that encodes an enzymatic activity that confers resistance to an antibiotic or drug upon the cell in which the selectable marker is expressed.

The term “regulatory element” as used herein refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

The term “host cells” generally refers to prokaryotic or eukaryotic hosts which are transformed or transfected with vectors constructed using recombinant DNA techniques known in the art. Transformed host cells are capable of either replicating vectors encoding the protein variants or expressing the desired protein variant. In the case of vectors which encode the pre- or pro-form of the protein variant, such variants, when expressed, are typically secreted from the host cell into the host cell medium.

The term “introduced” in the context of inserting a nucleic acid sequence into a cell, means transformation, transduction, or transfection. Means of transformation include protoplast transformation, calcium chloride precipitation, electroporation, naked DNA, and the like as known in the art. (See, Chang and Cohen [1979] Mol. Gen. Genet. 168:111-115; Smith et al. [1986] Appl. Env. Microbiol. 51:634; and the review article by Ferrari et al., in Harwood, Bacillus, Plenum Publishing Corporation, pp. 57-72, 1989).

Any headings used herein are provided for convenience and should not be construed as limitations. The description included under one heading may apply to the specification as a whole.

Variants, compositions and methods disclosed herein relate to a recombinant mannanase, comprising one or more modifications, wherein such variants are generated through conventional molecular biology techniques (see, e.g., Sambrook et al, Molecular Cloning: Cold Spring Harbor Laboratory Press). In one embodiment, the variant mannanase comprises one or more modifications selected from at least one substitution, at least one deletion, and at least one insertion. In some embodiments, the modification comprises a combination of mutations, such as, for example, a combination of at least one substitution and at least one deletion, at least one deletion and at least one insertion, at least one insertion and at least one substitution, or at least one substitution, at least one deletion, and at least one insertion.

In one embodiment, mannanase variants are provided, where the mannanase variants comprise one or more amino acid substitutions at one or more positions selected from 32, 72, 161 and 172 wherein the amino acid positions of the variant are numbered by correspondence with the amino acid sequence of SEQ ID NO: 1, and wherein the variant has at least 80% identity to the amino acid sequence of SEQ ID NO: 1.

In one embodiment, the mannanase variant is a mannanase variant comprising an amino acid substitution selected from the group consisting of 19D, 32Y, 34D, 72V, 93Q, 131S, 136P, 139R, 161G, 172F, 225N/Q, 259D, 261D/E and 276W, wherein the amino acid positions of the variant are numbered by correspondence with the amino acid sequence of SEQ ID NO: 1, and wherein the variant has at least 80% identity to the amino acid sequence of SEQ ID NO: 1.

In one embodiment, the mannanase variant is a mannanase variant comprising two or more amino acid substitutions selected from the group consisting of 19D, 32Y, 72V, 93Q, 131S, 136P, 139R, 161G, 168T, 172F, 225N/Q, 259D/E, 261D/E and 276W, wherein the amino acid positions of the variant are numbered by correspondence with the amino acid sequence of SEQ ID NO: 1, and wherein the variant has at least 80% identity to the amino acid sequence of SEQ ID NO: 1.

In one embodiment, the mannanase variant is a variant comprising amino acid substitutions selected from the group consisting of 19D-276W, 32Y-259D, 93Q-276W, 131S-276W, 136P-276W, 139R-276W, 161G-276W, 225N/Q-276W, 259D/E-276W, and 261D/E-276W

In one embodiment, the mannanase variant is a variant comprising amino acid substitutions selected from the group consisting of 19D-131S-276W, 32Y-261D-276W, 32Y-259D-276W, 32Y-172F-259D, 168T-259D-276W, 259D-261E-276W, and 259Q-261E-276W.

In one embodiment, the mannanase variant is a mannanase variant comprising amino acid substitutions selected from the group consisting of Y061W-G259D-R261E-F276W, Y061W-T062E-G259D-R261E-F276W, Y061W-V228T-G259D-R261E-F276W, V228T-G259D-R261E-F276W, F032Y-G259D-R261E-F276W, F032Y-Y061W-Y167F-P168S-G259D-R261E-F276W, F032Y-Y061W-G259D-R261E-F276W, F032Y-Y061W-T062E-G259D-R261E-F276W, F032Y-T062E-R261D-F276W, F032Y-T062E-G259D-F276W, T062E-G259D-R261E-F276W, V059S-Q060L-G259D-R261E-F276W, V059S-Q060L-Y061W-G259D-R261E-F276W, V059S-Q060L-T062E-G259D-R261E-F276W, T062E-V228T-G259D-R261E-F276W, F032Y-T062E-G259D-R261E-F276W, F032Y-T062E-G259D-R261E-F276W-T284E, E019D-A068S-T131S-F276W, F032Y-T062E-G259D-F276W-T284E, F032Y-T062E-V228T-G259D-F276W-T284E, F032Y-T062E-I072V-G259D-F276W-T284E, F032Y-T062E-A068S-G259D-F276W-T284E, F032Y-Y061W-T062E-G259D-F276W-T284E, F032Y-V059S-Q060L-T062E-G259D-F276W-T284E, N010T-F032Y-T062E-G259D-F276W-T284E, F032Y-T062E-Y167F-P168S-V228T-G259D-F276W-T284E, F032Y-T062E-I072V-V228T-G259D-F276W-T284E, F032Y-T062E-1072V-Y167F-P168S-G259D-F276W-T284E, F032Y-T062E-A068S-V228T-G259D-F276W-T284E, F032Y-Y061W-T062E-V228T-G259D-F276W-T284E, F032Y-Y061W-T062E-Y167F-P168S-G259D-F276W-T284E, F032Y-Y061W-T062E-I072V-G259D-F276W-T284E, F032Y-Y061W-T062E-A068S-G259D-F276W-T284E, F032Y-V059S-Q060L-T062E-V228T-G259D-F276W-T284E, F032Y-V059S, Q060L-T062E-Y167F-P168S-G259D-F276W-T284E, F032Y-V059S-Q060L-T062E-I072V-G259D-F276W-T284E, F032Y-V059S-Q060L-T062E-A068S-G259D-F276W-T284E, F032Y-V059S-Q060L-Y061W-T062E-G259D-F276W-T284E, N010T-F032Y-T062E-I072V-G259D-F276W-T284E, N010T-F032Y-T062E-A068S-G259D-F276W-T284E, N010T-F032Y-Y061W-T062E-G259D-F276W-T284E, N010T-F032Y-V059S-Q060L-T062E-G259D-F276W-T284E, F032Y-T062E-I072V-Y167F-P168S-V228T-G259D-F276W-T284E, F032Y-T062E-A068S-Y167F-P168S-V228T-G259D-F276W-T284E, F032Y-T062E-A068S-I072V-Y167F-P168S-G259D-F276W-T284E, F032Y-Y061W-T062E-Y167F-P168S-V228T-G259D-F276W-T284E, F032Y-Y061W-T062E-I072V-V228T-G259D-F276W-T284E, F032Y-Y061W-T062E-I072V-Y167F-P168S-G259D-F276W-T284E, F032Y-Y061W-T062E-A068S-V228T-G259D-F276W-T284E, F032Y-Y061W-T062E-A068S-Y167F-P168S-G259D-F276W-T284E, F032Y-Y061W-T062E-A068S-I072V-G259D-F276W-T284E, F032Y-V059S-Q060L-T062E-Y167F-P168S-V228T-G259D-F276W-T284E, F032Y-V059S-Q060L-T062E-I072V-V228T-G259D-F276W-T284E, F032Y-V059S-Q060L-T062E-1072V-Y167F-P168S-G259D-F276W-T284E, F032Y-V059S-Q060L-T062E-A068S-V228T-G259D-F276W-T284E, F032Y-V059S-Q060L-T062E-A068S-Y167F-P168S-G259D-F276W-T284E, F032Y-V059S-Q060L-T062E-A068S-I072V-G259D-F276W-T284E, F032Y-V059S-Q060L-Y061W-T062E-V228T-G259D-F276W-T284E, F032Y-V059S-Q060L-Y061W-T062E-Y167F-P168S-G259D-F276W-T284E, F032Y-V059S-Q060L-Y061W-T062E-I072V-G259D-F276W-T284E, F032Y-V059S-Q060L-Y061W-T062E-A068S-G259D-F276W-T284E, N010T-F032Y-T062E-I072V-V228T-G259D-F276W-T284E, N010T-F032Y-T062E-A068S-V228T-G259D-F276W-T284E, N010T-F032Y-T062E-A068S-I072V-G259D-F276W-T284E, N010T-F032Y-Y061W-T062E-V228T-G259D-F276W-T284E, N010T-F032Y-Y061W-T062E-Y167F-P168S-G259D-F276W-T284E, N010T-F032Y-Y061W-T062E-1072V-G259D-F276W-T284E, N010T-F032Y-V059S-Q060L-T062E-Y167F-P168S-G259D-F276W-T284E, N010T-F032Y-V059S-Q060L-T062E-1072V-G259D-F276W-T284E, F032Y-T062E-A068S-I072V-Y167F-P168S-V228T-G259D-F276W-T284E, F032Y-Y061W-T062E-I072V-Y167F-P168S-V228T-G259D-F276W-T284E, F032Y-Y061W-T062E-A068S-Y167F-P168S-V228T-G259D-F276W-T284E, F032Y-Y061W-T062E-A068S-I072V-V228T-G259D-F276W-T284E, F032Y-Y061W-T062E-A068S-I072V-Y167F-P168S-G259D-F276W-T284E, F032Y-V059S-Q060L-T062E-I072V-Y167F-P168S-V228T-G259D-F276W-T284E, F032Y-V059S-Q060L-T062E-A068S-Y167F-P168S-V228T-G259D-F276W-T284E, F032Y-V059S-Q060L-T062E-A068S-I072V-V228T-G259D-F276W-T284E, F032Y-V059S-Q060L-T062E-A068S-1072V-Y167F-P168S-G259D-F276W-T284E, F032Y-V059S-Q060L-Y061W-T062E-Y167F-P168S-V228T-G259D-F276W-T284E, F032Y-V059S-Q060L-Y061W-T062E-1072V-V228T-G259D-F276W-T284E, F032Y-V059S-Q060L-Y061W-T062E-I072V-Y167F-P168S-G259D-F276W-T284E, F032Y-V059S-Q060L-Y061W-T062E-A068S-V228T-G259D-F276W-T284E, F032Y-V059S-Q060L-Y061W-T062E-A068S-Y167F-P168S-G259D-F276W-T284E, F032Y-V059S-Q060L-Y061W-T062E-A068S-I072V-G259D-F276W-T284E, N010T-F032Y-T062E-I072V-Y167F-P168S-V228T-G259D-F276W-T284E, N010T-F032Y-T062E-A068S-Y167F-P168S-V228T-G259D-F276W-T284E, N010T-F032Y-T062E-A068S-I072V-V228T-G259D-F276W-T284E, N010T-F032Y-T062E-A068S-I072V-Y167F-P168S-G259D-F276W-T284E, N010T-F032Y-Y061W-T062E-Y167F-P168S-V228T-G259D-F276W-T284E, N010T-F032Y-Y061W-T062E-I072V-V228T-G259D-F276W-T284E, N010T-F032Y-Y061W-T062E-I072V-Y167F-P168S-G259D-F276W-T284E, N010T-F032Y-Y061W-T062E-A068S-Y167F-P168S-G259D-F276W-T284E, N010T-F032Y-Y061W-T062E-A068S-I072V-G259D-F276W-T284E, N010T-F032Y-V059S-Q060L-T062E-Y167F-P168S-V228T-G259D-F276W-T284E, N010T-F032Y-V059S-Q060L-T062E-1072V-V228T-G259D-F276W-T284E, N010T-F032Y-V059S-Q060L-T062E-1072V-Y167F-P168S-G259D-F276W-T284E, N010T-F032Y-V059S-Q060L-T062E-A068S-V228T-G259D-F276W-T284E, N010T-F032Y-V059S-Q060L-T062E-A068S-1072V-G259D-F276W-T284E, N010T-F032Y-V059S-Q060L-Y061W-T062E-V228T-G259D-F276W-T284E, N010T-F032Y-V059S-Q060L-Y061W-T062E-Y167F-P168S-G259D-F276W-T284E, N010T-F032Y-V059S-Q060L-Y061W-T062E-A068S-G259D-F276W-T284E, F032Y-Y061W-T062E-A068S-1072V-Y167F-P168S-V228T-G259D-F276W-T284E, F032Y-V059S-Q060L-T062E-A068S-I072V-Y167F-P168S-V228T-G259D-F276W-T284E, F032Y-V059S-Q060L-Y061W-T062E-I072V-Y167F-P168S-V228T-G259D-F276W-T284E, F032Y-V059S-Q060L-Y061W-T062E-A068S-Y167F-P168S-V228T-G259D-F276W-T284E, F032Y-V059S-Q060L-Y061W-T062E-A068S-1072V-V228T-G259D-F276W-T284E, F032Y-V059S-Q060L-Y061W-T062E-A068S-1072V-Y167F-P168S-G259D-F276W-T284E, N010T-F032Y-T062E-A068S-I072V-Y167F-P168S-V228T-G259D-F276W-T284E, N010T-F032Y-Y061W-T062E-I072V-Y167F-P168S-V228T-G259D-F276W-T284E, N010T-F032Y-Y061W-T062E-A068S-Y167F-P168S-V228T-G259D-F276W-T284E, N010T-F032Y-Y061W-T062E-A068S-1072V-V228T-G259D-F276W-T284E, N010T-F032Y-V059S-Q060L-T062E-I072V-Y167F-P168S-V228T-G259D-F276W-T284E, N010T-F032Y-V059S-Q060L-T062E-A068S-Y167F-P168S-V228T-G259D-F276W-T284E, N010T-F032Y-V059S-Q060L-T062E-A068S-1072V-V228T-G259D-F276W-T284E, N010T-F032Y-V059S-Q060L-T062E-A068S-I072V-Y167F-P168S-G259D-F276W-T284E, N010T-F032Y-V059S-Q060L-Y061W-T062E-Y167F-P168S-V228T-G259D-F276W-T284E, N010T-F032Y-V059S-Q060L-Y061W-T062E-I072V-Y167F-P168S-G259D-F276W-T284E, N010T-F032Y-V059S-Q060L-Y061W-T062E-A068S-Y167F-P168S-G259D-F276W-T284E, N010T-F032Y-V059S-Q060L-Y061W-T062E-A068S-I072V-G259D-F276W-T284E, N010T-F032Y-Y061W-T062E-A068S-I072V-Y167F-P168S-V228T-G259D-F276W-T284E, N010T-F032Y-V059S-Q060L-T062E-A068S-1072V-Y167F-P168S-V228T-G259D-F276W-T284E, N010T-F032Y-V059S-Q060L-Y061W-T062E-1072V-Y167F-P168S-V228T-G259D-F276W-T284E, N010T-F032Y-V059S-Q060L-Y061W-T062E-A068S-Y167F-P168S-V228T-G259D-F276W-T284E, N010T-F032Y-V059S-Q060L-Y061W-T062E-A068S-I072V-V228T-G259D-F276W-T284E, N010T-F032Y-V059S-Q060L-Y061W-T062E-A068S-1072V-Y167F-P168S-G259D-F276W-T284E and N010T-F032Y-V059S-Q060L-Y061W-T062E-A068S-I072V-Y167F-P168S-V228T-G259D-F276W-T284E, F032Y-Y061W-T062E-G259D-R261E-F276W-T284E, F032Y-T062E-A068S-G259D-R261E-F276W-T284E, F032Y-T062E-T131S-G259D-R261E-F276W-T284E, F032Y-T062E-N150D-G259D-R261E-F276W-T284E, F032Y-T062E-P168S-G259D-R261E-F276W-T284E, F032Y-T062E-L235K-G259D-R261E-F276W-T284E, N010T-F032Y-Y061W-T062E-P168A-V228T-G259D-F276W-T284E, N010T-F032Y-Y061W-T062E-Y167F-P168S-V228T-G259E-F276W-T284E, F032Y-Y061W-T062E-T131S-Y167F-P168S-V228T-G259D-F276W-T284E, N010T-F032Y-Y061W-T062E-A136P-Y167F-P168S-V228T-G259D-F276W-T284E, N010T-F032Y-Y061W-T062E-K093Q-Y167F-P168S-V228T-G259D-F276W-T284E, N010T-F032Y-Y061W-T062E-Y167F-P168S-V228T-L235K-G259D-F276W-T284E, N010T-F032Y-Y061W-T062E-D139R-Y167F-P168S-V228T-G259D-F276W-T284E, N010T-F032Y-Y061W-T062E-N150D-Y167F-P168S-V228T-G259D-F276W-T284E, N010T-F032Y-Y061W-T062E-Y167F-P168A-V228T-G259D-F276W-T284E, N010T-F032Y-Y061W-T062E-T131S-Y167F-P168S-V228T-G259D-F276W-T284E, N010T-F032Y-Y061W-T062E-P168S-V228T-G259D-F276W-T284E, N010T-F032Y-Y061W-T062E-A068S-Y167F-P168A-G259D-F276W-T284E, and N010T-F032Y-Y061W-T062E-A068S-Y167F-P168A-G259E-F276W-T284E, wherein the amino acid positions of the variant are numbered by correspondence with the amino acid sequence of SEQ ID NO: 1, and wherein the variant has at least 80% identity to the amino acid sequence of SEQ ID NO: 1.

In one embodiment, the mannanase variant is a mannanase variant described herein, wherein the variant comprises an amino acid sequence having at least 85%, 90%, 91%, 92%, 93%, 94%, 95%, 96%, 97%, 98%, or 99% sequence identity to the amino acid sequence of SEQ ID NO: 1.

In one embodiment, the mannanase variant is a mannanase variant described herein, wherein said variant is derived from a parent or reference polypeptide with 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1.

In one embodiment, the mannanase variant is a mannanase variant described herein, wherein said variant has improved stability when compared to a parent or reference mannanase.

In one embodiment, the mannanase variant is a mannanase variant described herein, wherein said variant has equal or improved cleaning performance in a detergent when compared to a parent or reference mannanase.

In one embodiment, the mannanase variant is a mannanase variant described herein, wherein the mannanase variant has mannanase activity.

Another embodiment is directed to a mannanase variant comprising an amino acid sequence having at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to the amino acid sequence of SEQ ID NO:1-6.

In one embodiment, the reference polypeptide is selected from SEQ ID NO: 1 or SEQ ID NO: 2. In another embodiment, one or more mannanase variant described herein has at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% amino acid sequence identity to the amino acid sequence of SEQ ID NO:1, SEQ ID NO:2, SEQ ID NO:3, SEQ ID NO:4, SEQ ID NO:5, and/or SEQ ID NO:6.

In some embodiments, the mannanase variants or recombinant polypeptides described herein are isolated. In other embodiments, the mannanase variants described herein are endo-β-mannanases. In further embodiments, the mannanase variants described herein have mannanase activity. In still other embodiments, the mannanase variants described herein have mannanase activity in the presence of a surfactant. In some embodiments, the mannanase activity is activity on mannan gum, locust bean gum galactomannan, and/or konjac glucomannan. In additional embodiments, the mannanase variants described herein have cleaning activity in a detergent composition. Still other embodiments are directed to mannanase variants or recombinant polypeptides that have mannanase activity in the presence of a protease. Further embodiments are directed to mannanase variants or recombinant polypeptides that hydrolyze a substrate selected from the group consisting of guar gum, locust bean gum, and combinations thereof. In some embodiments, the mannanase variants or recombinant polypeptides described herein do not comprise a carbohydrate-binding module.

In some embodiments, the mannanase variant has enzymatic activity over a broad range of pH conditions. In certain embodiments, the mannanase variant has enzymatic activity from a pH of about 4.0 to about 11.0. In further embodiments, the mannanase variants or recombinant polypeptides have at least 50%, 60%, 70%, 80%, 90%, 95%, or 100% mannanase activity at a pH of from about 4.0 to about 11.0, about 4.5 to about 9.0, about 5.5 to about 8.5, or about 6.0 to about 7.5.

In a still further embodiment, the mannanase variants or recombinant polypeptides have mannanase activity at a temperature ranging from about 20 °C to about 90 °C, about 30 °C to about 80 °C, about 20 °C to about 50 °C, or about 30 °C to about 66 °C. In certain embodiments, the mannanase variants or recombinant polypeptides have at least 50%, 60%, 70%, 80%, 90%, 95%, or 100% mannanase activity at a temperature range from about 20 °C to about 90 °C, about 30 °C to about 80 °C, about 20 °C to about 50 °C, or about 30 °C to about 66 °C.

Yet still further embodiments are directed to mannanase variants or recombinant polypeptides described herein, wherein the variant retains at least 70% of its maximal mannanase activity at a pH range of 4.5-9.0, 5.5-8.5, or 6.0-7.5. Some embodiments are directed to mannanase variants or recombinant polypeptides described herein, wherein the variant retains at least 70% of its maximal mannanase activity at a pH above 3.0, 3.5, 4.0 or 4.5 or at a pH below 9.0, 9.5, or 10.0.

In some embodiments one or more mannanase variant described herein has one or more improved property when compared to a reference polypeptide, wherein the improved property is selected from improved stability in the presence of protease, improved stability in detergent or buffer, improved cleaning performance, and improved aged cleaning performance. Aged cleaning performance refers to the difference in stain removal measured for a sample of aged test sample (where the enzyme is pre-incubated in detergent for an extended period of time such as 3-4 weeks at an elevated temperature such as 37 °C) compared to the ‘fresh’ stain cleaning for the same enzyme (no pre-incubation). Thus, an enzyme with improved aged cleaning performance displays a smaller difference between the aged and freshly prepared samples when compared to the same evaluation carried out with a reference/parent enzyme.

In another embodiment, one or more mannanase variant described herein has one or more improved property when compared to a reference polypeptide, wherein the improved property is selected from improved stability in the presence of protease, improved stability in detergent or buffer, improved cleaning performance, wherein the reference polypeptide is selected from SEQ ID NO: 1 or 2.

In some embodiments, the mannanase variants or recombinant polypeptides are substantially identical to SEQ ID NO: 1 or 2, meaning that they can contain amino acid substitutions, insertions, or deletions that do not significantly affect the structure, function, or expression of the variant or polypeptide. Such mannanase variants or recombinant polypeptides include those designed only to circumvent the present description.

In some embodiments, the mannanase variants have 1,4-β-D-mannosidic hydrolase activity, which includes mannanase, endo-1,4-β-D-mannanase, exo-1,4-β-D-mannanase galactomannanase, and/or glucomannanase activity. 1,4-β-D-mannosidic hydrolase activity can be determined and measured using the assays described herein, or by other assays known in the art. In some embodiments, a polypeptide of the present invention has activity in the presence of a detergent composition.

In some embodiments, the mannanase variants described herein are produced as an N- and/or C-terminal fusion protein, for example, to aid in extraction, detection and/or purification and/or to add functional properties to the variant or recombinant polypeptides or active fragments thereof. Examples of fusion protein partners include, but are not limited to, glutathione-S-transferase (GST), 6×His, GAL4 (DNA binding and/or transcriptional activation domains), FLAG, MYC, BCE103 (WO 2010/044786), or other tags well known to anyone skilled in the art. In some embodiments, a proteolytic cleavage site is provided between the fusion protein partner and the protein sequence of interest to allow removal of fusion protein sequences. Preferably, the fusion protein does not hinder the activity of the mannanase variants or recombinant polypeptides described herein.

In some embodiments, the mannanase variants or recombinant polypeptides described herein are fused to a functional domain including a leader peptide, propeptide, one or more binding domain (modules) and/or a catalytic domain. Suitable binding domains include, but are not limited to, carbohydrate-binding modules (CBM) of various specificities, providing increased affinity to carbohydrate components present during the application of the mannanase variants or recombinant polypeptides described herein. As described herein, the CBM and catalytic domain of a polypeptide of the present invention are operably linked.

A CBM is defined as a contiguous amino acid sequence within a carbohydrate-active enzyme with a discreet fold having carbohydrate-binding activity. A few exceptions are CBMs in cellulosomal scaffold in proteins and rare instances of independent putative CBMs. The requirement of CBMs existing as modules within larger enzymes sets this class of carbohydrate-binding proteins apart from other non-catalytic sugar binding proteins such as lectins and sugar transport proteins. CBMs were previously classified as cellulose-binding domains (CBDs) based on the initial discovery of several modules that bound cellulose (Tomme et al., Eur J Biochem, 170:575-581, 1988; and Gilkes et al., J Biol Chem, 263:10401-10407, 1988). However, additional modules in carbohydrate-active enzymes are continually being found that bind carbohydrates other than cellulose, yet otherwise meet the CBM criteria, hence the need to reclassify these polypeptides using more inclusive terminology. Previous classification of cellulose-binding domains was based on amino acid similarity. Groupings of CBDs were called “Types” and numbered with Roman numerals (e.g. Type I or Type II CBDs). In keeping with the glycoside hydrolase classification, these groupings are now called families and numbered with Arabic numerals. Families 1 to 13 are the same as Types I to XIII (Tomme et al., in Enzymatic Degradation of Insoluble Polysaccharides (Saddler, J. N. & Penner, M., eds.), Cellulose-binding domains: classification and properties. pp. 142-163, American Chemical Society, Washington, 1995). A detailed review on the structure and binding modes of CBMs can be found in Boraston et al., Biochem J, 382:769-81, 2004. The family classification of CBMs is expected to aid in the identification of CBMs, predict binding specificity, aid in identifying functional residues, reveal evolutionary relationships, and possibly be predictive of polypeptide folds. Because the fold of proteins is better conserved than their sequences, some of the CBM families can be grouped into superfamilies or clans. The current CBM families are 1-63. CBDs are found at the N- and C-termini of proteins or are internal. Enzyme hybrids are known in the art (See e.g., WO90/00609 and WO95/16782) and may be prepared by transforming into a host cell a DNA construct comprising at least a fragment of DNA encoding the cellulose-binding domain ligated, with or without a linker, to a DNA sequence encoding a mannanase variant described herein and growing the host cell to express the fused gene.

Enzyme hybrids may be described by the following formula: CBM-MR-X or X-MR-CBM, wherein CBM is the N-terminal or the C-terminal region of an amino acid sequence corresponding to at least the carbohydrate-binding module; MR is the middle region (the linker), and may be a bond, or a short linking group of from about 2 to about 100 carbon atoms, from about 2 to about 40 carbon atoms, from about 2 to about 100 amino acids, or from about 2 to about 40 amino acids; and X is an N-terminal or C-terminal region of a mannanase variant described herein that has mannanase catalytic activity. In addition, a mannanase may contain more than one CBM or other module(s)/domain(s) of non-glycolytic function. The terms “module” and “domain” are used interchangeably in the present disclosure.

Further non-limiting examples of catalytic domains include: cellulases; hemicellulases, such as xylanase; exo-mannanases; glucanases; arabinases; galactosidases; pectinases; and/or other activities such as proteases, lipases, acid phosphatases and/or others or functional fragments thereof. Fusion proteins are optionally linked to a mannanase variant described herein through a linker sequence that simply joins the mannanase variant and the fusion domain without significantly affecting the properties of either component, or the linker optionally has a functional importance for the intended application.

In some embodiments, the enzymes are mannanase variants as provided herein in combination with one or more additional enzymes selected from the group consisting of acyl transferases, amylases, alpha-amylases, beta-amylases, alpha-galactosidases, arabinases, arabinosidases, aryl esterases, beta-galactosidases, beta-glucanases, carrageenases, catalases, cellulases, chondroitinases, cutinases, dispersins, DNAses (also known as nucleases or dispersins), endo-glucanases, endo-beta-mannanases, exo-beta-mannanases, esterases, exo-mannanases, galactanases, glucoamylases, hemicellulases, hexosaminidase, hyaluronidases, keratinases, laccases, lactases, ligninases, lipases, lipolytic enzymes, lipoxygenases, lysozyme, mannanases, metalloproteases, nucleases, oxidases, oxidoreductases, pectate lyases, pectin acetyl esterases, pectinases, pentosanases, perhydrolases, peroxidases, PETases, phenoloxidases, phosphatases, phospholipases, phosphodiesterases, phytases, polyesterases, polygalacturonases, proteases, pullulanases, reductases, rhamnogalacturonases, tannases, transglutaminases, Xanthan lyases, Xanthanases, xylan acetyl-esterases, xylanases, xylosidases, and combinations thereof or mixture thereof.

In some embodiments, a mannanase variant described herein is fused to a signal peptide for directing the extracellular secretion of the variant or polypeptide. For example, in certain embodiments, the signal peptide is the native signal peptide of the mannanase variant described herein. In other embodiments, the signal peptide is a non-native signal peptide such as the B. subtilis AprE signal peptide.

In some embodiments, a polypeptide of the present invention is expressed in a heterologous organism, i.e., an organism other than Paenibacillus spp. Exemplary heterologous organisms are Gram (+) bacteria such as B. subtilis, B. licheniformis, B. lentus, B. brevis, Geobacillus (formerly Bacillus) stearothermophilus, B. alkalophilus, B. amyloliquefaciens, B. coagulans, B. circulans, B. lautus, B. megaterium, B. thuringiensis, S. lividans, or S. murinus; Gram (−) bacteria such as E. coli; yeast such as Saccharomyces spp. or Schizosaccharomyces spp., e.g. S. cerevisiae; and filamentous fungi such as Aspergillus spp., e.g., A. oryzae or A. niger, and T. reesei. Methods for transforming nucleic acids into these organisms are well known in the art. A suitable procedure for transformation of Aspergillus host cells is described in EP238023.

In particular embodiments, a mannanase variant described herein is expressed in a heterologous organism as a secreted polypeptide, in which case, the compositions and method encompass a method for expressing the variant as a secreted polypeptide in a heterologous organism.

Further embodiments are directed to methods of producing a mannanase variant described herein comprising: stably transforming a host cell with an expression vector comprising a polynucleotide encoding the mannanase variant; culturing the transformed host cell under suitable conditions to produce the mannanase variant; and recovering the mannanase variant.

Yet another embodiment is directed to a polynucleotide that encodes a mannanase variant described herein. In one aspect, the polynucleotide is contained in an expression vector contained in a heterologous organism, such as those identified, herein. The polynucleotide may be operably-linked to regulatory elements (e.g., a promoter, terminator, enhancer, and the like) to assist in expressing the encoded variants or recombinant polypeptides described herein.

Some embodiments are directed to a polynucleotide that encodes a mannanase variant having at least 59%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to the amino acid sequence of SEQ ID NO:1-6. In some embodiments, the polynucleotide is codon-optimized for expression in a different host, mutated to introduce cloning sites, or otherwise altered to add functionality.

In some embodiments, the polynucleotide that encodes a mannanase described herein is fused downstream of a coding sequence of a signal peptide that directs the extracellular secretion of variant. Expression vectors may be provided in a heterologous host cell suitable for expressing a variant described herein, or suitable for propagating the expression vector prior to introducing it into a suitable host cell.

DNA that encodes a mannanase variant described herein can be chemically synthesized from published sequences or obtained directly from host cells harboring the gene (e.g., by cDNA library screening or PCR amplification). In some embodiments, a polynucleotide is included in an expression cassette and/or cloned into a suitable expression vector by standard molecular cloning techniques. Such expression cassettes or vectors contain sequences that assist initiation and termination of transcription (e.g., promoters and terminators), and generally contain a selectable marker.

The expression cassette or vector is introduced into a suitable expression host cell, which then expresses the corresponding mannanase variant described herein. Particularly suitable expression hosts are bacterial expression host genera including Escherichia (e.g., E. coli), Pseudomonas (e.g., P. fluorescens or P. stutzerei), Proteus (e.g., P. mirabilis), Ralstonia (e.g., R. eutropha), Streptomyces, Staphylococcus (e.g., S. carnosus), Lactococcus (e.g., L. lactis), or Bacillus (subtilis, megaterium, licheniformis, etc.). Also particularly suitable are yeast expression hosts such as S. cerevisiae, S. pombe, Y. lipolytica, H. polymorpha, K. lactis or P. pastoris. Especially suited are fungal expression hosts such as C. lucknowense, Aspergillus (e.g., A. oryzae, A. niger, A. nidulans, etc.) or T. reesei. Also suited are mammalian expression hosts such as mouse (e.g., NS0), Chinese Hamster Ovary (CHO) or Baby Hamster Kidney (BHK) cell lines. Other eukaryotic hosts such as insect cells or viral expression systems (e.g., bacteriophages such as M13, T7 phage or Lambda, or viruses such as Baculovirus) are also suitable for producing a mannanase variant described herein.

Promoters and/or signal sequences associated with secreted proteins in a particular host of interest are candidates for use in the heterologous production and secretion of mannanases in that host or in other hosts. As an example, in filamentous fungal systems, the promoters that drive the genes for cellobiohydrolase I (cbh1), glucoamylase A (glaA), TAKA-amylase (amyA), xylanase (exlA), the gpd-promoter cbh1, cbhll, endoglucanase genes EGI-EGV, Cel61B, Cel74A, egl1-egl5, gpd promoter, Pgk1, pki1, EF-1alpha, tef1, cDNA1 and hex1 are particularly suitable and can be derived from a number of different organisms (e.g., A. niger, T. reesei, A. oryzae, A. awamori and A. nidulans). In some embodiments, the polynucleotide is recombinantly associated with a polynucleotide encoding a suitable homologous or heterologous signal sequence that leads to secretion of a mannanase variant described herein into the extracellular (or periplasmic) space, thereby allowing direct detection of enzyme activity in the cell supernatant (or periplasmic space or lysate). Particularly suitable signal sequences for E. coli, other Gram negative bacteria and other organisms known in the art include those that drive expression of the HlyA, DsbA, Pbp, PhoA, PelB, OmpA, OmpT or M13 phage Gill genes. For B. subtilis, Gram-positive organisms and other organisms known in the art, particularly suitable signal sequences further include those that drive expression of AprE, NprB, Mpr, AmyA, AmyE, Blac, SacB, and for S. cerevisiae or other yeast, include the killer toxin, Bar1, Suc2, Mating factor alpha, Inu1A or Ggplp signal sequence. Signal sequences can be cleaved by a number of signal peptidases, thus removing them from the rest of the expressed protein. In some embodiments, the rest of the polypeptide is expressed alone or as a fusion with other peptides, tags or proteins located at the N- or C-terminus (e.g., 6×His, HA or FLAG tags). Suitable fusions include tags, peptides or proteins that facilitate affinity purification or detection (e.g., BCE103, 6×His, HA, chitin binding protein, thioredoxin or FLAG tags), as well as those that facilitate expression, secretion or processing of the target mannanase. Suitable processing sites include enterokinase, STE13, Kex2 or other protease cleavage sites for cleavage in vivo or in vitro.

A mannanase variant described herein can be introduced into expression host cells by a number of transformation methods including, but not limited to, electroporation, lipid-assisted transformation or transfection (“lipofection”), chemically mediated transfection (e.g., CaCl and/or CaP), lithium acetate-mediated transformation (e.g., of host-cell protoplasts), biolistic “gene gun” transformation, PEG-mediated transformation (e.g., of host-cell protoplasts), protoplast fusion (e.g., using bacterial or eukaryotic protoplasts), liposome-mediated transformation, Agrobacterium tumefaciens, adenovirus or other viral or phage transformation or transduction.

Alternatively, a mannanase variant described herein can be expressed intracellularly. Optionally, after intracellular expression of the enzyme variants, or secretion into the periplasmic space using signal sequences such as those mentioned above, a permeabilisation or lysis step can be used to release the polypeptide into the supernatant. The disruption of the membrane barrier is effected by the use of mechanical means such as ultrasonic waves, pressure treatment (French press), cavitation or the use of membrane-digesting enzymes such as lysozyme or enzyme mixtures. As a further alternative, the polynucleotides encoding a mannanase variant described herein can be expressed by use of a suitable cell-free expression system. In cell-free systems, the polynucleotide of interest is typically transcribed with the assistance of a promoter, but ligation to form a circular expression vector is optional. In other embodiments, RNA is exogenously added or generated without transcription and translated in cell free systems.

Another embodiment is directed to a cleaning composition comprising a mannanase variant and methods for using such compositions in cleaning applications. Cleaning applications include, but are not limited to, laundry or textile cleaning, laundry or textile softening, dishwashing (manual and automatic), stain pre-treatment, and the like. Particular applications are those where mannans (e.g., locust bean gum, guar gum, etc.) are a component of the soils or stains to be removed.

Cleaning compositions typically include an effective amount of a mannanase variant described herein, e.g., at least 0.0001 weight percent, from about 0.0001 to about 1, from about 0.001 to about 0.5, from about 0.01 to about 0.1 weight percent, or even from about 0.1 to about 1 weight percent, or more. An effective amount of a mannanase variant in the cleaning composition results in the mannanase variant having enzymatic activity sufficient to hydrolyze a mannan-containing substrate, such as locust bean gum, guar gum, or combinations thereof.

Some embodiments are directed to a cleaning composition in a form selected from powder, liquid, granular, bar, solid, semi-solid, gel, paste, emulsion, tablet, capsule, unit dose, sheet, and foam. In some embodiments, the cleaning composition is a detergent composition. In other embodiments, the cleaning composition or detergent composition is selected from a laundry detergent, a fabric softening detergent, a dishwashing detergent, a medical instrument cleaning detergent, and a hard-surface cleaning detergent.

In some embodiments, the cleaning compositions comprising one or more mannanase variant described herein is a detergent composition selected from the group consisting of a laundry detergent, a fabric softening detergent, a dishwashing detergent, a medical instrument cleaning detergent, and a hard-surface cleaning detergent.

In some embodiments, the invention is directed to detergent compositions comprising at least two proteases in combination with one or more additional cleaning composition components such as, but not limiting to, a liquid laundry composition described in WO2022106404.

In some embodiments, the one or more mannanase variant described herein can be part of, or added to, a liquid laundry detergent composition such as, but not limiting to, a liquid laundry composition described in U.S. Pat. No. 11,046,919B2, WO2021/223552, WO2022/167251, WO2022/074037, WO2021/123184, WO2021/037895, WO2022/10372, WO2020/264077, WO2022/106404 and/or WO2017/54983; a compacted liquid laundry composition (U.S. Pat. No. 10,683,474B2); a water-soluble unit dose article comprising a fatty alkyl ester alkoxylate non-ionic surfactant and an alkoxylated alcohol non-ionic surfactant (US20220162523A1); a liquid laundry detergent composition comprising improved alkylbenzenesulfonate surfactants (WO2021/108307); a liquid laundry detergent composition comprising benzyl benzoate (WO2020/223959); and/or a detergent compositions containing a branched surfactant (WO2021/247801), a water-soluble unit dose article comprising an amphiphilic graft polymer and a polyester terephthalate (WO2019/032257).

In some embodiments, the cleaning compositions comprising one or more mannanase variant described herein is a liquid laundry detergent composition containing alkyl ether carboxylic acids, betaines, anionic surfactant, non-ionic surfactant for providing softening benefits (WO2013/087286).

In some embodiments, the cleaning compositions comprising one or more mannanase variant described herein is a liquid laundry detergent composition containing sulfite radical scavengers, protease stabilizers/inhibitors or combinations thereof (WO2022/157311)

In some embodiments, the cleaning composition comprising one or more mannanase variant described herein is a liquid laundry detergent composition as described in US20210317387A1, WO2021/219296, WO2021/127662, WO2021/041685, U.S. Pat. No. 11,208,619, US20220186144

In some embodiments, the cleaning composition comprising one or more mannanase variant described herein is a liquid laundry detergent composition comprising dispersin variants, such as but limiting to a liquid laundry detergent composition described in US20210317387A1.

In some embodiments, the cleaning composition comprising one or more mannanase variant described herein is a liquid laundry detergent composition is a highly alkaline textile washing agent, such as but limiting to a liquid laundry detergent composition described in WO2021/219296

In some embodiments, the cleaning composition comprising one or more mannanase variant described herein is a liquid laundry detergent composition is a low density unit dose detergent with encapsulated fragrance, such as but limiting to a detergent composition described in WO2021/127662.

In some embodiments, the cleaning composition comprising one or more mannanase variant described herein is a liquid laundry detergent composition containing polyethylene glycol and an organic acid, such as but limiting to, a detergent composition described in WO2021/041685.

In some embodiments, the cleaning composition comprising one or more mannanase variant described herein is a detergent composition containing polyethylene glycol and an organic acid, such as but limiting to, a detergent composition described in WO2021/041685.

In some embodiments, the cleaning composition comprising one or more mannanase variant described herein is a detergent composition with effect on protein stains, such as but limiting to, a detergent composition described in U.S. Pat. No. 11,208,619.

In some embodiments, the cleaning composition comprising one or more mannanase variant described herein is a detergent composition containing soil release polymers, such as but limiting to, a detergent composition described in US20220186144.

Unless otherwise noted, all component or composition levels provided herein are made in reference to the active level of that component or composition, and are exclusive of impurities, for example, residual solvents or by-products, which may be present in commercially available sources. Enzyme component weights are based on total active protein. All percentages and ratios are calculated by weight unless otherwise indicated. All percentages and ratios are calculated based on the total composition unless otherwise indicated. In exemplified detergent compositions, the enzymes levels are expressed by pure enzyme by weight of the total composition and unless otherwise specified, the detergent ingredients are expressed by weight of the total compositions.

In some embodiments, the cleaning compositions described herein further comprise a surfactant. In some embodiments, the surfactant is selected from a non-ionic, ampholytic, semi-polar, anionic, cationic, zwitterionic, and combinations and mixtures thereof. In yet a further embodiment, the surfactant is selected from an anionic surfactant, a cationic surfactant, a zwitterionic surfactant, and combinations thereof. In some embodiments, the cleaning compositions described herein comprise from about 0.1% to about 60%, about 1% to about 50%, or about 5% to about 40% surfactant by weight of the composition. Exemplary surfactants include, but are not limited to sodium dodecylbenzene sulfonate, C12-14 pareth-7, C12-15 pareth-7, sodium C12-15 pareth sulfate, C14-15 pareth-4, sodium laureth sulfate (e.g., Steol CS-370), sodium hydrogenated cocoate, C12 ethoxylates (Alfonic 1012-6, Hetoxol LA7, Hetoxol LA4), sodium alkyl benzene sulfonates (e.g., Nacconol 90G), and combinations and mixtures thereof. Anionic surfactants include but are not limited to linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. Nonionic surfactants include but are not limited to alcohol ethoxylate (AEO or AE), carboxylated alcohol ethoxylates, nonylphenol ethoxylate, alkylpolyglycoside, alkyldimethylamine oxide, ethoxylated fatty acid monoethanolamide, fatty acid monoethanolamide, polyhydroxy alkyl fatty acid amide (e.g., as described in WO92/06154), polyoxyethylene esters of fatty acids, polyoxyethylene sorbitan esters (e.g., TWEENs), polyoxyethylene alcohols, polyoxyethylene isoalcohols, polyoxyethylene ethers (e.g., TRITONs and BRIJ), polyoxyethylene esters, polyoxyethylene-p-tert-octylphenols or octylphenyl-ethylene oxide condensates (e.g., NONIDET P40), ethylene oxide condensates with fatty alcohols (e.g., LUBROL), polyoxyethylene nonylphenols, polyalkylene glycols (SYNPERONIC F108), sugar-based surfactants (e.g., glycopyranosides, thioglycopyranosides), and combinations and mixtures thereof.

In a further embodiment, the detergent compositions disclosed herein further comprise a surfactant mixture that includes, but is not limited to 5-15% anionic surfactants, <5% nonionic surfactants, cationic surfactants, phosphonates, soap, enzymes, perfume, butylphenyl methylpropionate, geraniol, zeolite, polycarboxylates, hexyl cinnamal, limonene, cationic surfactants, citronellol, and benzisothiazolinone.

The cleaning compositions described herein may additionally include one or more detergent builders or builder systems, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, a tarnish inhibitor, an optical brightener, a fabric conditioner, and a perfume. The cleaning compositions described herein may also include additional enzymes selected from proteases, amylases, cellulases, lipases, pectin degrading enzymes, xyloglucanases, or additional carboxylic ester hydrolases.

In some embodiments, the cleaning composition described herein further comprises from about 1%, from about 3% to about 60% or even from about 5% to about 40% builder by weight of the cleaning composition. Builders may include, but are not limited to, the alkali metals, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metals, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

In some embodiments, the builders form water-soluble hardness ion complexes (e.g., sequestering builders), such as citrates and polyphosphates (e.g., sodium tripolyphosphate and sodium tripolyphosphate hexahydrate, potassium tripolyphosphate, and mixed sodium and potassium tripolyphosphate, etc.). Any suitable builder can find use in the compositions described herein, including those known in the art (See, e.g., EP 2100949).

As indicated herein, in some embodiments, the cleaning compositions described herein further comprise an adjunct ingredient including, but not limited to surfactants, builders, bleaches, bleach activators, bleach catalysts, other enzymes, enzyme stabilizing systems, chelants, optical brighteners, soil release polymers, dye transfer agents, dye transfer inhibiting agents, catalytic materials, hydrogen peroxide, sources of hydrogen peroxide, preformed peracids, polymeric dispersing agents, clay soil removal agents, structure elasticizing agents, dispersants, suds suppressors, dyes, perfumes, colorants, filler salts, hydrotropes, photoactivators, fluorescers, fabric conditioners, hydrolyzable surfactants, solvents, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See, e.g., U.S. Pat. Nos. 6,610,642; 6,605,458; 5,705,464; 5,710,115; 5,698,504; 5,695,679; 5,686,014; and 5,646,101). In some embodiments, one or more adjunct is incorporated for example, to assist or enhance cleaning performance, for treatment of the substrate to be cleaned, or to modify the aesthetics of the cleaning composition as is the case with perfumes, colorants, dyes or the like. Any such adjunct ingredient is in addition to the mannanase variant described herein. The precise nature of these additional components, and levels of incorporation thereof, will depend on the physical form of the composition and the nature of the cleaning operation for which it is to be used.

In embodiments in which one or more adjunct ingredient is not compatible with the mannanase variant, suitable methods can be employed to keep the cleaning adjunct ingredient and mannanases separated (i.e., not in contact with each other) until combination of the two components is appropriate. Such separation methods include any suitable method known in the art (e.g., gelcaps, encapsulation, tablets, physical separation, etc.). The specific selection of suitable adjunct ingredients is readily made by considering the surface, item, or fabric to be cleaned, and the desired form of the composition for the cleaning conditions during use (e.g., through the wash detergent use).

The cleaning compositions described herein are advantageously employed for example, in laundry applications, hard surface cleaning, dishwashing applications, as well as cosmetic applications. Furthermore, the polypeptides of the present invention may find use in granular and liquid compositions.

A mannanase variant described herein may also find use in cleaning additive products. In some embodiments, the additive is packaged in a dosage form suitable for addition to a cleaning process. In some embodiments, the additive is packaged in a dosage form for addition to a cleaning process where a source of peroxygen is employed and increased bleaching effectiveness is desired. Any suitable single unit dosage form finds use with the present disclosure, including but not limited to pills, tablets, gelcaps, or other single unit dosage form such as pre-measured powders or liquids. In some embodiments, filler(s) or carrier material(s) are included to increase the volume of such compositions. Suitable filler or carrier materials include, but are not limited to various salts of sulfate, carbonate, and silicate as well as talc, clay, and the like. Suitable filler or carrier materials for liquid compositions include, but are not limited to water or low molecular weight primary and secondary alcohols including polyols and diols. Examples of such alcohols include, but are not limited to methanol, ethanol, propanol, and isopropanol. In some embodiments, the compositions contain from about 5% to about 90% of such materials. Acidic fillers find use to reduce pH. Alternatively, in some embodiments, the cleaning additive includes one or more adjunct ingredients.

In one embodiment, the cleaning composition or cleaning additive contains an effective amount of a mannanase variant described herein, optionally in combination with other mannanases and/or additional enzymes. In certain embodiments, the additional enzymes include, but are not limited to, at least one enzyme selected from of acyl transferases, amylases, alpha-amylases, beta-amylases, alpha-galactosidases, arabinases, arabinosidases, aryl esterases, beta-galactosidases, beta-glucanases, carrageenases, catalases, cellulases, chondroitinases, cutinases, dispersins, DNAses (also known as nucleases or dispersins), endo-glucanases, endo-beta-mannanases, exo-beta-mannanases, esterases, exo-mannanases, galactanases, glucoamylases, hemicellulases, hexosaminidase, hyaluronidases, keratinases, laccases, lactases, ligninases, lipases, lipolytic enzymes, lipoxygenases, lysozyme, mannanases, metalloproteases, nucleases, oxidases, oxidoreductases, pectate lyases, pectin acetyl esterases, pectinases, pentosanases, perhydrolases, peroxidases, PETases, phenoloxidases, phosphatases, phospholipases, phosphodiesterases, phytases, polyesterases, polygalacturonases, proteases, pullulanases, reductases, rhamnogalacturonases, tannases, transglutaminases, Xanthan lyases, Xanthanases, xylan acetyl-esterases, xylanases, xylosidases, and combinations thereof or mixture thereof. In further embodiments, the cleaning compositions or cleaning additives described herein further comprise a protease and/or amylase.

The cleaning compositions herein are typically formulated such that, during use in aqueous cleaning operations, the wash water will have a pH of from about 3.0 to about 11. Liquid product formulations are typically formulated to have a neat pH from about 5.0 to about 9.0. Granular laundry products are typically formulated to have a pH from about 8.0 to about 11.0. Techniques for controlling pH at recommended usage levels include the use of buffers, alkalis, acids, etc., and are well known to those skilled in the art.

Suitable low pH cleaning compositions typically have a neat pH of from about 3.0 to about 5.0 or even from about 3.5 to about 4.5. Low pH cleaning compositions are typically free of surfactants that hydrolyze in such a pH environment. Such surfactants include sodium alkyl sulfate surfactants that comprise at least one ethylene oxide moiety or even from about 1 to about 16 moles of ethylene oxide. Such cleaning compositions typically comprise a sufficient amount of a pH modifier, such as sodium hydroxide, monoethanolamine, or hydrochloric acid, to provide such cleaning composition with a neat pH of from about 3.0 to about 5.0. Such compositions typically comprise at least one acid stable enzyme. In some embodiments, the compositions are liquids, while in other embodiments, they are solids. The pH of such liquid compositions is typically measured as a neat pH. The pH of such solid compositions is measured as a 10% solids solution of the composition wherein the solvent is distilled water. In these embodiments, all pH measurements are taken at 20 °C, unless otherwise indicated.

Suitable high pH cleaning compositions typically have a neat pH of from about 9.0 to about 11.0, or even a neat pH of from 9.5 to 10.5. Such cleaning compositions typically comprise a sufficient amount of a pH modifier, such as sodium hydroxide, monoethanolamine, or hydrochloric acid, to provide such cleaning composition with a neat pH of from about 9.0 to about 11.0. Such compositions typically comprise at least one base-stable enzyme. In some embodiments, the compositions are liquids, while in other embodiments, they are solids. The pH of such liquid compositions is typically measured as a neat pH. The pH of such solid compositions is measured as a 10% solids solution of said composition wherein the solvent is distilled water. In these embodiments, all pH measurements are taken at 20 °C, unless otherwise indicated.

In some embodiments, the mannanase variant is in the form of an encapsulated particle to protect it from other components of the granular composition during storage. In addition, encapsulation is also a means of controlling the availability of the mannanase variant during the cleaning process. In some embodiments, encapsulation enhances the performance of the mannanase variant and/or additional enzymes. In this regard, the mannanase variant is encapsulated with any suitable encapsulating material known in the art. Typically, the encapsulating material is water-soluble and/or water-dispersible. In some embodiments, the encapsulating material has a glass transition temperature (Tg) of 0 °C. or higher. Glass transition temperature is described in more detail in WO97/11151. The encapsulating material is typically selected from carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (See, e.g., EP0922499 and U.S. Pat. Nos. 4,977,252; 5,354,559; and 5,935,826). In some embodiments, the encapsulating material is a microsphere made from plastic such as thermoplastics, acrylonitrile, methacrylonitrile, polyacrylonitrile, polymethacrylonitrile, and mixtures thereof; commercially available microspheres that find use include, but are not limited to those supplied by EXPANCEL® (Stockviksverken, Sweden), and PM6545, PM6550, PM7220, PM7228, EXTENDOSPHERES®, LUXSIL®, Q-CEL®, and SPHERICEL® (PQ Corp., Valley Forge, PA).

The term “granular composition” refers to a conglomeration of discrete solid, macroscopic particles. Powders are a special class of granular material due to their small particle size, which makes them more cohesive and more easily suspended.

Concentrations of detergent compositions in typical wash solutions throughout the world vary from less than about 800 ppm of detergent composition (“low detergent concentration geographies”), for example about 667 ppm in Japan, to between about 800 ppm to about 2000 ppm (“medium detergent concentration geographies”), for example about 975 ppm in U.S. and about 1500 ppm in Brazil, to greater than about 2000 ppm (“high detergent concentration geographies”), for example about 4500 ppm to about 5000 ppm in Europe and about 6000 ppm in high suds phosphate builder geographies.

In some embodiments, the detergent compositions described herein may be utilized at a temperature of from about 10 °C to about 60 °C, or from about 20 °C to about 60 °C, or from about 30 °C to about 60 °C, from about 40 °C to about 60 °C, from about 40 °C to about 55 °C, or all ranges within 10 °C to 60 °C. In some embodiments, the detergent compositions described herein are used in “cold water washing” at temperatures of from about 10 °C to about 40 °C, or from about 20 °C to about 30 °C, from about 15 °C to about 25 °C, from about 15 °C to about 35 °C, or all ranges within 10 °C to 40 °C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million (parts per million converted to grains per U.S. gallon is ppm #divided by 17.1 equals grains per gallon) of hardness minerals.

TABLE 1
Water Hardness Levels

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

In some embodiments, a mannanase variant described herein is comparable in wash performance to commercially available mannanases. In some embodiments, a mannanase variant described herein exhibits enhanced wash performance as compared to commercially available mannanases. In some embodiments, a mannanase variant described herein exhibits enhanced cleaning capabilities under various conditions, and/or enhanced chelator stability. In addition, a mannanase variant described herein may find use in cleaning compositions that do not include detergents, again either alone or in combination with builders and stabilizers.

In addition to the mannanase variants described herein, any other suitable mannanase may find use in the compositions described herein either alone or in combination with the variants described herein. Suitable mannanases include, but are not limited to, mannanases of the GH26 family of glycosyl hydrolases, mannanases of the GH5 family of glycosyl hydrolases, acidic mannanases, neutral mannanases, and alkaline mannanases. Examples of alkaline mannanases include those described in U.S. Pat. Nos. 6,060,299; 6,566,114; and 6,602,842; and WO9535362, WO9964573, WO9964619, and WO2015022428. Additionally, suitable mannanases include, but are not limited to those of animal, plant, fungal, or bacterial origin. Chemically or genetically modified mutants are encompassed by the present disclosure.

Examples of useful mannanases include Bacillus endo-β-mannanases such as B. subtilis endo-β-mannanase (See, e.g., U.S. Pat. No. 6,060,299 and WO9964573), Bacillus sp. 1633 endo-β-mannanase (See, e.g., U.S. Pat. No. 6,566,114 and WO9964619), Bacillus sp. AAI12 endo-β-mannanase (See, e.g., U.S. Pat. No. 6,566,114 and WO9964619), B. sp. AA349 endo-β-mannanase (See, e.g., U.S. Pat. No. 6,566,114 and WO9964619), B. agaradhaerens NCIMB 40482 endo-β-mannanase (See, e.g., U.S. Pat. No. 6,566,114 and WO9964619), B. halodurans endo-β-mannanase, B. clausii endo-β-mannanase (See, e.g., U.S. Pat. No. 6,566,114 and WO9964619), B. licheniformis endo-β-mannanase (See, e.g., U.S. Pat. No. 6,566,114 and WO9964619A1), Humicola endo-β-mannanases such as H. insolens endo-β-mannanase (See, e.g., U.S. Pat. No. 6,566,114 and WO9964619), and Caldocellulosiruptor endo-β-mannanases such as C. sp. endo-β-mannanase (See, e.g., U.S. Pat. No. 6,566,114 and WO9964619).

Furthermore, a number of identified mannanases (i.e., endo-β-mannanases and exo-β-mannanases) find use in some embodiments of the present disclosure, including but not limited to A. bisporus mannanase (See, Tang et al., [2001] Appl. Environ. Microbiol. 67:2298-2303), A. tamarii mannanase (See, Civas et al., [1984] Biochem. J. 219:857-863), A. aculeatus mannanase (See, Christgau et al., [1994] Biochem. Mol. Biol. Int. 33:917-925), A. awamori mannanase (See, Setati et al., [2001] Protein Express Purif. 21:105-114), A. fumigatus mannanase (See, Puchart et al., [2004] Biochimica et biophysica Acta. 1674:239-250), A. niger mannanase (See, Ademark et al., [1998] J. Biotechnol. 63:199-210), A. oryzae NRRL mannanase (See, Regalado et al., [2000] J. Sci. Food Agric. 80:1343-1350), A. sulphureus mannanase (See, Chen et al., [2007] J. Biotechnol. 128(3):452-461), A. terrus mannanase (See, Huang et al., [2007] Wei Sheng Wu Xue Bao. 47(2): 280-284), Paenibacillus and Bacillus spp. mannanase (See, U.S. Pat. No. 6,376,445.), Bacillus AM001 mannanase (See, Akino et al., [1989] Arch. Microbiol. 152:10-15), B. brevis mannanase (See, Araujo and Ward, [1990] J. Appl. Bacteriol. 68:253-261), B. circulans K-1 mannanase (See, Yoshida et al., [1998] Biosci. Biotechnol. Biochem. 62(3):514-520), B. polymyxa mannanase (See, Araujo and Ward, [1990] J. Appl. Bacteriol. 68:253-261), Bacillus sp JAMB-750 mannanase (See, Hatada et al., [2005] Extremophiles. 9:497-500), Bacillus sp. M50 mannanase (See, Chen et al., [2000] Wei Sheng Wu Xue Bao. 40:62-68), Bacillus sp. N 16-5 mannanase (See, Yanhe et al., [2004] Extremophiles 8:447-454), B. stearothermophilus mannanase (See, Talbot and Sygusch, [1990] Appl. Environ. Microbiol. 56:3505-3510), B. subtilis mannanase (See, Mendoza et al., [1994] World J. Microbiol. Biotechnol. 10:51-54), B. subtilis B36 mannanase (Li et al., [2006] Z. Naturforsch (C). 61:840-846), B. subtilis BM9602 mannanase (See, Cui et al., [1999] Wei Sheng Wu Xue Bao. 39(1):60-63), B. subtilis SA-22 mannanase (See, Sun et al., [2003] Sheng Wu Gong Cheng Xue Bao. 19(3):327-330), B. subtilis 168 mannanase (See, Helow and Khattab, [1996] Acta Microbiol. Immunol. Hung. 43:289-299), B. ovatus mannanase (See, Gherardini et al., [1987] J. Bacteriol. 169:2038-2043), B. ruminicola mannanase (See, Matsushita et al., [1991] J. Bacteriol. 173:6919-6926), C. cellulovorans mannanase (See, Sunna et al., [2000] Appl. Environ. Microbiol. 66:664-670), C. saccharolyticus mannanase (See, Morris et al., [1995] Appl. Environ. Microbiol. 61:2262-2269), C. saccharolyticum mannanase (See, Bicho et al., [1991] Appl. Microbiol. Biotechnol. 36:337-343), C. fimi mannanase (See, Stoll et al., [1999] Appl. Environ. Microbiol. 65(6):2598-2605), C. butyricum/beijerinckii mannanase (See, Nakajima and Matsuura, [1997] Biosci. Biotechnol. Biochem. 61:1739-1742), C. cellulolyticum mannanase (See, Perret et al., [2004] Biotechnol. Appl. Biochem. 40:255-259), C. tertium mannanase (See, Kataoka and Tokiwa, [1998] J. Appl. Microbiol. 84:357-367), C. thermocellum mannanase (See, Halstead et al., [1999] Microbiol. 145:3101-3108), D. thermophilum mannanase (See, Gibbs et al., [1999] Curr. Microbiol. 39(6):351-357), Flavobacterium sp. mannanase (See, Zakaria et al., [1998] Biosci. Biotechnol. Biochem. 62:655-660), G. pulmonata mannanase (See, Charrier and Rouland, [2001] J. Expt. Zool. 290:125-135), L. brevicula mannanase (See, Yamamura et al., [1996] Biosci. Biotechnol. Biochem. 60:674-676), L. esculentum mannanase (See, Filichkin et al., [2000] Plant Physiol. 134:1080-1087), P. curdlanolyticus mannanase (See, Pason and Ratanakhanokchai, [2006] Appl. Environ. Microbiol. 72:2483-2490), P. polymyxa mannanase (See, Han et al., [2006] Appl. Microbiol Biotechnol. 73(3):618-630), P. chrysosporium mannanase (See, Wymelenberg et al., [2005] J. Biotechnol. 118:17-34), Piromyces sp. mannanase (See, Fanutti et al., [1995] J. Biol. Chem. 270(49):29314-29322), P. insulars mannanase (See, Yamamura et al., [1993] Biosci. Biotechnol. Biochem. 7:1316-1319), P. fluorescens subsp. cellulosa mannanase (See, Braithwaite et al., [1995] Biochem J. 305:1005-1010), R. marinus mannanase (See, Politz et al., [2000] Appl. Microbiol. Biotechnol. 53 (6):715-721), S. rolfsii mannanase (See, Sachslehner et al., [2000] J. Biotechnol. 80:127-134), S. galbus mannanase (See, Kansoh and Nagieb, [2004] Anton. van. Leeuwenhoek. 85:103-114), S. lividans mannanase (See, Arcand et al., [1993] J. Biochem. 290:857-863), T. polysaccharolyticum mannanase (See, Cann et al., [1999] J. Bacteriol. 181:1643-1651), T. fusca mannanase (See, Hilge et al., [1998] Structure 6:1433-1444), T. maritima mannanase (See, Parker et al., [2001] Biotechnol. Bioeng. 75(3):322-333), T. neapolitana mannanase (See, Duffaud et al., [1997] Appl. Environ. Microbiol. 63:169-177), T. harzianum strain T4 mannanase (See, Franco et al., [2004] Biotechnol Appl. Biochem. 40:255-259), T. reesei mannanase (See, Stalbrand et al., [1993] J. Biotechnol. 29:229-242), and Vibrio sp. mannanase (See, Tamaru et al., [1997] J. Ferment. Bioeng. 83:201-205).

Additional suitable mannanases include commercially available endo-β-mannanases such as HEMICELL® (Chemgen); GAMANASE® and MANNAWAY®, (Novozymes A/S, Denmark); EFFECTENZ™ M 1000, PREFERENZ® M 100, PURABRITE™ and MANNASTAR™ (DuPont); and PYROLASE® 160 and PYROLASER 200 (Diversa).

In other embodiments, the composition described herein comprises one or more mannanase variant described herein and one or more additional enzyme. The one or more additional enzyme is selected from acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, galactanases, glucoamylases, hemicellulases, hyaluronidases, keratinases, laccases, lactases, ligninases, lipases, lipoxygenases, additional mannanases, metalloproteases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, and any combination or mixture thereof. Some embodiments are directed to a combination of enzymes (i.e., a “cocktail”) comprising conventional enzymes like amylase, lipase, cutinase, protease and/or cellulase in conjunction with one or more mannanase variant described herein and/or one or more additional mannanase.

In some embodiments, the cleaning compositions described herein further comprise a protease. In some embodiments, the composition comprises from about 0.00001% to about 10% protease by weight of the composition. In another embodiment, the cleaning composition comprises from about 0.0001% to about 10%, about 0.001% to about 5%, about 0.001% to about 2%, or about 0.005% to about 0.5% protease by weight of the composition.

In one embodiment, the protease is a serine protease. Suitable proteases include those of animal, vegetable or microbial origin. In some embodiments, the protease is a microbial protease. In other embodiments, the protease is a chemically or genetically modified mutant. In another embodiment, the protease is an alkaline microbial protease or a trypsin-like protease. Exemplary alkaline proteases include subtilisins derived from, for example, Bacillus (e.g., subtilisin, lentus, amyloliquefaciens, gibsonii, subtilisin Carlsberg, subtilisin 309, sp. TY-145, subtilisin 147 and subtilisin 168). Exemplary additional proteases include but are not limited to those described in WO92/21760, WO95/23221, WO2008/010925, WO09/149200, WO09/149144, WO09/149145, WO 10/056640, WO10/056653, WO2010/0566356, WO11/072099, WO2011/13022, WO11/140364, WO12/151534, WO2015/038792, WO2015/089447, WO2015/089441, WO2015/143360, WO2016/061438, WO2016/069548, WO2016/069544, WO2016/069557, WO2016/069563, WO2016/069569, WO2016/069552, WO2016/145428, US Publ. No. 2008/0090747, U.S. Pat. Nos. 5,801,039, 5,340,735, 5,500,364, 5,855,625, RE 34,606, U.S. Pat. Nos. 5,955,340, 5,700,676, 6,312,936, 6,482,628, 8,530,219, U.S. Provisional Appl Nos. 62/331,282, 62/332,417, 62/343,618, and 62/351,649, and PCT Appl Nos. PCT/US16/32514 and PCT/US2016/038245, as well as metalloproteases described in WO1999014341, WO1999033960, WO1999014342, WO1999034003, WO2007044993, WO2009058303, WO 2009058661, WO2014071410, WO2014194032, WO2014194034, WO 2014194054, and WO 2014/194117. Exemplary proteases include, but are not limited to trypsin (e.g., of porcine or bovine origin) and the Fusarium protease described in WO89/06270. Exemplary commercial proteases include, but are not limited to MAXATASE®, MAXACAL™, MAXAPEM™, OPTICLEAN®, OPTIMASE®, PROPERASE®, PURAFECT®, PURAFECT® OXP, PURAMAX™, EXCELLASE™, PREFERENZ™ proteases (e.g. P100, P110, P280), EFFECTENZ™ proteases (e.g. P1000, P1050, P2000), EXCELLENZ™ proteases (e.g. P1000), ULTIMASE®, and PURAFAST™ (DuPont); ALCALASE®, BLAZE®, BLAZE® EVITY®, BLAZER EVITY® 16L, CORONASE®, SAVINASE®, SAVINASE® ULTRA, SAVINASE® EVITY®, SAVINASE® EVERIS®, PRIMASE®, DURAZYM™, POLARZYME®, OVOZYME®, KANNASE®, LIQUANASE®, LIQUANASE EVERIS®, NEUTRASE®, PROGRESS UNO®, RELASE® and ESPERASE® (Novozymes); BLAP™ and BLAP™ variants (Henkel); LAVERGY™ PRO 104 L (BASF), and KAP (B. alkalophilus subtilisin (Kao)).

In some embodiments, the cleaning compositions described herein further comprise a suitable amylase. In one embodiment, the composition comprises from about 0.00001% to about 10%, about 0.0001% to about 10%, about 0.001% to about 5%, about 0.001% to about 2%, or about 0.005% to about 0.5% amylase by weight of the composition. Any amylase (e.g., alpha and/or beta) suitable for use in alkaline solutions may be useful to include in such composition. An exemplary amylase can be a chemically or genetically modified mutant. Exemplary amylases include, but are not limited to those of bacterial or fungal origin, such as, for example, amylases described in GB 1,296,839, WO9100353, WO9402597, WO94183314, WO9510603, WO9526397, WO9535382, WO9605295, WO9623873, WO9623874, WO 9630481, WO9710342, WO9741213, WO9743424, WO9813481, WO 9826078, WO9902702, WO 9909183, WO9919467, WO9923211, WO9929876, WO9942567, WO 9943793, WO9943794, WO 9946399, WO0029560, WO0060058, WO0060059, WO0060060, WO 0114532, WO0134784, WO 0164852, WO0166712, WO0188107, WO0196537, WO02092797, WO 0210355, WO0231124, WO 2004055178, WO2004113551, WO2005001064, WO2005003311, WO 2005018336, WO2005019443, WO2005066338, WO2006002643, WO2006012899, WO2006012902, WO2006031554, WO 2006063594, WO2006066594, WO2006066596, WO2006136161, WO 2008000825, WO2008088493, WO2008092919, WO2008101894, WO2008/112459, WO2009061380, WO2009061381, WO 2009100102, WO2009140504, WO2009149419, WO 2010/059413, WO 2010088447, WO2010091221, WO2010104675, WO2010115021, WO10115028, WO2010117511, WO 2011076123, WO2011076897, WO2011080352, WO2011080353, WO 2011080354, WO2011082425, WO2011082429, WO 2011087836, WO2011098531, WO2013063460, WO2013184577, WO 2014099523, WO2014164777, and WO2015077126. Exemplary commercial amylases include, but are not limited to AMPLIFY®, AMPLIFY PRIME® DURAMYL®, TERMAMYL®, FUNGAMYL®, STAINZYME®, STAINZYME PLUS® STAINZYME PLUS®, STAINZYME ULTRA® EVITY®, and BAN™ (Novozymes); EFFECTENZ™ S 1000, POWERASE™, PREFERENZ™ S 100, PREFERENZ™ S 110, EXCELLENZ™ S 2000, RAPIDASE® and MAXAMYL® P (DuPont).

In some embodiments, the cleaning compositions described herein further comprise a suitable pectin degrading enzyme. As used herein, “pectin degrading enzyme(s)” encompass arabinanase (EC 3.2.1.99), galactanases (EC 3.2.1.89), polygalacturonase (EC 3.2.1.15) exo-polygalacturonase (EC 3.2.1.67), exo-poly-alpha-galacturonosidase (EC 3.2.1.82), pectin lyase (EC 4.2.2.10), pectin esterase (EC 3.1.1.11), pectate lyase (EC 4.2.2.2), exo-polygalacturonate lyase (EC 4.2.2.9) and hemicellulases such as endo-1,3-β-xylosidase (EC 3.2.1.32), xylan-1,4-β-xylosidase (EC 3.2.1.37) and α-L-arabinofuranosidase (EC 3.2.1.55). Pectin degrading enzymes are natural mixtures of the above mentioned enzymatic activities. Pectin enzymes therefore include the pectin methylesterases which hydrolyse the pectin methyl ester linkages, polygalacturonases which cleave the glycosidic bonds between galacturonic acid molecules, and the pectin transeliminases or lyases which act on the pectic acids to bring about non-hydrolytic cleavage of α-1,4 glycosidic linkages to form unsaturated derivatives of galacturonic acid.

Suitable pectin degrading enzymes include those of plant, fungal, or microbial origin. In some embodiments, chemically or genetically modified mutants are included. In some embodiments, the pectin degrading enzymes are alkaline pectin degrading enzymes, i.e., enzymes having an enzymatic activity of at least 10%, at least 25%, or at least 40% of their maximum activity at a pH of from about 7.0 to about 12. In certain other embodiments, the pectin degrading enzymes are enzymes having their maximum activity at a pH of from about 7.0 to about 12. Alkaline pectin degrading enzymes are produced by alkalophilic microorganisms e.g., bacterial, fungal, and yeast microorganisms such as Bacillus species. In some embodiments, the microorganisms are B. firmus, B. circulans, and B. subtilis as described in JP 56131376 and JP 56068393. Alkaline pectin decomposing enzymes may include but are not limited to galacturan-1,4-α-galacturonidase (EC 3.2.1.67), poly-galacturonase activities (EC 3.2.1.15, pectin esterase (EC 3.1.1.11), pectate lyase (EC 4.2.2.2) and their iso enzymes. Alkaline pectin decomposing enzymes can be produced by the Erwinia species. In some embodiments, the alkaline pectin decomposing enzymes are produced by E. chrysanthemi, E. carotovora, E. amylovora, E. herbicola, and E. dissolvens as described in JP 59066588, JP 63042988, and in World J. Microbiol. Biotechnol. (8, 2, 115-120) 1992. In certain other embodiments, the alkaline pectin enzymes are produced by Bacillus species as disclosed in JP 73006557 and Agr. Biol. Chem. (1972), 36 (2) 285-93. In some embodiments, the cleaning compositions described herein further comprise about 0.00001% to about 10%, about 0.0001% to about 10%, about 0.001% to about 5%, about 0.001% to about 2%, or about 0.005% to about 0.5% of pectin degrading enzyme by weight of the composition.

In some other embodiments, the cleaning compositions described herein further comprise a suitable xyloglucanase. Suitable xyloglucanases include, but are not limited to those of plant, fungal, or bacterial origin. Chemically or genetically modified mutants are included in some embodiments. As used herein, “xyloglucanase(s)” encompass the family of enzymes described by Vincken and Voragen at Wageningen University [Vincken et al (1994) Plant Physiol., 104, 99-107] and are able to degrade xyloglucans as described in Hayashi et al (1989) Annu. Rev. Plant. Physiol. Plant Mol. Biol., 40, 139-168. Vincken et al demonstrated the removal of xyloglucan coating from cellulose of the isolated apple cell wall by a xyloglucanase purified from Trichoderma viride (endo-IV-glucanase). This enzyme enhances the enzymatic degradation of cell wall-embedded cellulose and work in synergy with pectic enzymes. Rapidase LIQ+ from DSM contains a xyloglucanase activity. In some embodiments, the cleaning compositions described herein further comprise from about 0.00001% to about 10%, about 0.0001% to about 10%, about 0.001% to about 5%, about 0.001% to about 2%, or about 0.005% to about 0.5% xyloglucanase by weight of the composition. In certain other embodiments, xyloglucanases for specific applications are alkaline xyloglucanases, i.e., enzymes having an enzymatic activity of at least 10%, at least 25%, or at least 40% of its maximum activity at a pH ranging from 7 to 12. In certain other embodiments, the xyloglucanases are enzymes having a maximum activity at a pH of from about 7.0 to about 12.

In some further embodiments, the detergent compositions described herein further comprise a suitable cellulase. In one embodiment, the composition comprises from about 0.00001% to about 10%, 0.0001% to about 10%, about 0.001% to about 5%, about 0.001% to about 2%, or about 0.005% to about 0.5% cellulase by weight of the composition. Any suitable cellulase may find use in a composition described herein. An exemplary cellulase can be a chemically or genetically modified mutant. Exemplary cellulases include, but are not limited to those of bacterial or fungal origin, such as, for example, those described in WO2005054475, WO2005056787, U.S. Pat. Nos. 7,449,318, 7,833,773, 4,435,307; EP 0495257; and U.S. Provisional Appl. No. 62/296,678. Exemplary commercial cellulases include, but are not limited to, CELLUCLEAN®, CELLUZYME®, CAREZYME®, ENDOLASE®, RENOZYME®, and CAREZYME® PREMIUM (Novozymes); REVITALENZ™ 100, REVITALENZ™ 200/220, and REVITALENZ® 2000 (DuPont); and KAC-500 (B)™ (Kao Corporation). In some embodiments, cellulases are incorporated as portions or fragments of mature wild-type or variant cellulases, wherein a portion of the N-terminus is deleted (see, e.g., U.S. Pat. No. 5,874,276).

In still further embodiments, the detergent compositions described herein further comprise a suitable lipase. In some embodiments, the composition comprises from about 0.00001% to about 10%, about 0.0001% to about 10%, about 0.001% to about 5%, about 0.001% to about 2%, or about 0.005% to about 0.5% lipase by weight composition. An exemplary lipase can be a chemically or genetically modified mutant. Exemplary lipases include, but are not limited to, e.g., those of bacterial or fungal origin, such as, e.g., H. lanuginosa lipase (see, e.g., EP 258068 and EP 305216), T. lanuginosus lipase (see, e.g., WO 2014/059360 and WO2015/010009), Rhizomucor miehei lipase (see, e.g., EP 238023), Candida lipase, such as C. antarctica lipase (e.g., C. antarctica lipase A or B) (see, e.g., EP 214761), Pseudomonas lipases such as P. alcaligenes and P. pseudoalcaligenes lipase (see, e.g., EP 218272), P. cepacia lipase (see, e.g., EP 331376), P. stutzeri lipase (see, e.g., GB 1,372,034), P. fluorescens lipase, Bacillus lipase (e.g., B. subtilis lipase (Dartois et al., Biochem. Biophys. Acta 1131:253-260 (1993)), B. stearothermophilus lipase (see, e.g., JP 64/744992), and B. pumilus lipase (see, e.g., WO 91/16422)). Exemplary cloned lipases include, but are not limited to Penicillium camembertii lipase (See, Yamaguchi et al., Gene 103:61-67 (1991)), Geotricum candidum lipase (See, Schimada et al., J. Biochem., 106:383-388 (1989)), and various Rhizopus lipases, such as, R. delemar lipase (See, Hass et al., Gene 109:117-113 (1991)), R. niveus lipase (Kugimiya et al., Biosci. Biotech. Biochem. 56:716-719 (1992)) and R. oryzae lipase. Other lipolytic enzymes, such as cutinases, may also find use in one or more composition described herein, including, but not limited to, e.g., cutinase derived from Pseudomonas mendocina (see, WO 88/09367) and/or Fusarium solani pisi (see, WO90/09446). Exemplary commercial lipases include, but are not limited to M1 LIPASE™, LUMA FAST™, and LIPOMAX™ (DuPont); LIPEX®, LIPOCLEAN®, LIPOLASE® and LIPOLASE® ULTRA (Novozymes); and LIPASE P™ (Amano Pharmaceutical Co. Ltd).

In some embodiments, cleaning compositions described herein further comprise peroxidases in combination with hydrogen peroxide or a source thereof (e.g., a percarbonate, perborate or persulfate). In some alternative embodiments, oxidases are used in combination with oxygen. Both types of enzymes are used for “solution bleaching” (i.e., to prevent transfer of a textile dye from a dyed fabric to another fabric when the fabrics are washed together in a wash liquor), preferably together with an enhancing agent (See, e.g., WO94/12621 and WO95/01426). Suitable peroxidases/oxidases include, but are not limited to those of plant, bacterial or fungal origin. Chemically or genetically modified mutants are included in some embodiments. In some embodiments, the cleaning compositions of the present disclosure further comprise from about 0.00001% to about 10%, about 0.0001% to about 10%, about 0.001% to about 5%, about 0.001% to about 2%, about 0.005% to about 0.5% of peroxidase and/or oxidase by weight of the composition.

In some embodiments, cleaning compositions described herein further comprise additional enzymes, including but not limited to perhydrolases (See, e.g., WO 05/056782). Some embodiments are directed to mixtures of one or more above mentioned protease, amylase, lipase, mannanase, and/or cellulase.

Some embodiments are directed to cleaning compositions such as, for example, those described in U.S. Pat. No. 6,605,458. In some embodiments, the cleaning compositions described herein are compact granular fabric cleaning compositions, while in other embodiments the composition is a granular fabric cleaning composition useful in the laundering of colored fabrics. In further embodiments, the composition is a granular fabric cleaning composition which provides softening through the wash capacity, and in additional embodiments the composition is a heavy duty liquid (HDL) fabric cleaning composition. In other embodiments, the cleaning compositions described herein are fabric cleaning compositions such as, for example, those described in U.S. Pat. Nos. 6,610,642 and 6,376,450. In an alternative embodiment, the cleaning compositions described herein are suitable hard surface cleaning compositions. Suitable hard surface cleaning compositions include, for example, those described in U.S. Pat. Nos. 6,610,642; 6,376,450; and 6,376,450. In yet further embodiments, the cleaning compositions described herein are dishwashing compositions. In some further embodiments, the compositions described herein are oral care compositions such as, for example, those described in U.S. Pat. Nos. 6,376,450 and 6,605,458. The formulations and descriptions of the compounds and cleaning adjunct materials contained in the aforementioned U.S. Pat. Nos. 6,376,450; 6,605,458; and 6,610,642 find use with a polypeptide of the present invention.

In still further embodiments, the cleaning compositions described herein are fabric softening compositions such as, for example, those described in GB 400898, GB 514276, EP0011340, EP0026528, EP0242919, EP0299575, EP0313146, and U.S. Pat. No. 5,019,292.

The cleaning compositions described herein can be formulated into any suitable form and prepared by any process chosen by the formulator, non-limiting examples of which are described in U.S. Pat. Nos. 5,879,584; 5,691,297; 5,574,005; 5,569,645; 5,565,422; 5,516,448; 5,489,392; and 5,486,303. When a low pH cleaning composition is desired, the pH of such composition is adjusted via the addition of a material such as monoethanolamine or an acidic material such as HCl.

In some embodiments, the cleaning compositions described herein are provided in unit dose form, including tablets, capsules, sachets, pouches, sheets, and multi-compartment pouches. In some embodiments, the unit dose format is designed to provide controlled release of the ingredients within a multi-compartment pouch (or other unit dose format). Suitable unit dose and controlled release formats are known in the art (See e.g., EP2100949, EP2100947, WO02/102955, WO04/111178, WO2013/165725, and U.S. Pat. Nos. 4,765,916 and 4,972,017). In some embodiments, the unit dose form is provided by tablets wrapped with a water-soluble film or water-soluble pouches.

In some embodiments, the cleaning compositions described herein further comprise at least one chelating agent. Suitable chelating agents may include, but are not limited to copper, iron, and/or manganese chelating agents, and mixtures thereof. In embodiments in which at least one chelating agent is used, the cleaning compositions of the present disclosure comprise from about 0.1% to about 15% or even from about 3.0% to about 10% chelating agent by weight of the cleaning composition.

In some still further embodiments, the cleaning compositions described herein further comprise at least one deposition aid. Suitable deposition aids include, but are not limited to, polyethylene glycol, polypropylene glycol, polycarboxylate, soil release polymers such as polyterephthalic acid, clays such as kaolinite, montmorillonite, attapulgite, illite, bentonite, halloysite, and mixtures thereof.

In some embodiments, the cleaning compositions described herein further comprise at least one anti-redeposition agent. In some embodiments, the anti-redeposition agent is a non-ionic surfactant, such as, for example, described in EP2100949. In some automatic dishwashing embodiments, non-ionic surfactants are used as surface modifiers, in particular for sheeting, to avoid filming and spotting and to improve shine.

In some embodiments, the cleaning compositions described herein further comprise one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, and polyvinylimidazoles, or mixtures thereof. In some embodiments, the cleaning compositions described herein comprise from about 0.0001% to about 10%, from about 0.01% to about 5%, or even from about 0.1% to about 3% dye transfer inhibiting agent by weight of the cleaning composition.

In some embodiments, the cleaning compositions described herein further comprise one or more silicates. In some such embodiments, sodium silicates (e.g., sodium disilicate, sodium metasilicate, and crystalline phyllosilicates) find use. In some embodiments, the cleaning compositions described herein comprise from about 1% to about 20% or from about 5% to about 15% silicate by weight of the composition.

In yet further embodiments, the cleaning compositions described herein further comprise one or more dispersant. Suitable water-soluble organic materials include, but are not limited to the homo- or co-polymeric acids or their salts, in which the polycarboxylic acid comprises at least two carboxyl radicals separated from each other by not more than two carbon atoms.

In some further embodiments, the enzymes used in the cleaning compositions are stabilized by any suitable technique. In some embodiments, the enzymes employed herein are stabilized by the presence of water-soluble sources of calcium and/or magnesium ions in the finished compositions. In some embodiments, the enzyme stabilizers include oligosaccharides, polysaccharides, and inorganic divalent metal salts, including alkaline earth metals, such as calcium salts. It is contemplated that various techniques for enzyme stabilization will find use in the present disclosure. For example, in some embodiments, the enzymes employed herein are stabilized by the presence of water-soluble sources of zinc (II), calcium (II), and/or magnesium (II) ions in the finished compositions, as well as other metal ions (e.g., barium (II), scandium (II), iron (II), manganese (II), aluminum (III), tin (II), cobalt (II), copper (II), nickel (II), and oxovanadium (IV)). Chlorides and sulfates also find use in some embodiments. Examples of suitable oligosaccharides and polysaccharides (e.g., dextrins) are known in the art (See, e.g., WO07/145964). In some embodiments, reversible protease inhibitors, such as boron-containing compounds (e.g., borate, 4-formyl phenyl boronic acid) and/or a peptide aldehyde U.S. Pat. No. 9,181,296B2) find use to further improve stability.

In some embodiments, the cleaning compositions described herein further comprise one or more bleach, bleach activator, and/or bleach catalyst. In some embodiments, the cleaning compositions described herein comprise inorganic and/or organic bleaching compound(s). Inorganic bleaches may include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Suitable salts include, for example, those described in EP2100949. Bleach activators are typically organic peracid precursors that enhance the bleaching action in the course of cleaning at temperatures of 60 °C. and below. Bleach activators suitable for use herein include compounds which, under perhydrolysis conditions, give aliphatic peroxycarboxylic acids having preferably from about 1 to about 10 carbon atoms, in particular from about 2 to about 4 carbon atoms, and/or optionally substituted perbenzoic acid. Suitable bleach activators include, for example, those described in EP2100949. Bleach catalysts typically include, for example, manganese triazacyclononane and related complexes, and cobalt, copper, manganese, and iron complexes, as well as those described in U.S. Pat. Nos. 4,246,612; 5,227,084; 4,810,410; and WO99/06521 and EP2100949.

In some embodiments, the cleaning compositions described herein further comprise one or more catalytic metal complex. In some embodiments, a metal-containing bleach catalyst finds use. In other embodiments, the metal bleach catalyst comprises a catalyst system comprising a transition metal cation of defined bleach catalytic activity (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra(methylenephosphonic acid) and water-soluble salts thereof are used (See, e.g., U.S. Pat. No. 4,430,243). In some embodiments, the cleaning compositions described herein are catalyzed by means of a manganese compound. Such compounds and levels of use are well known in the art (See, e.g., U.S. Pat. No. 5,576,282). In additional embodiments, cobalt bleach catalysts find use in the cleaning compositions described herein. Various cobalt bleach catalysts are known in the art (See, e.g., U.S. Pat. Nos. 5,597,936 and 5,595,967) and are readily prepared by known procedures.

In some additional embodiments, the cleaning compositions described herein further comprise a transition metal complex of a macropolycyclic rigid ligand (MRL). As a practical matter, and not by way of limitation, in some embodiments, the compositions and cleaning processes provided herein are adjusted to provide on the order of at least one part per hundred million of the active MRL species in the aqueous washing medium, and in other embodiments, provide from about 0.005 ppm to about 25 ppm, from about 0.05 ppm to about 10 ppm, or from about 0.1 ppm to about 5 ppm of the MRL in the wash liquor.

In some embodiments, the transition-metal in the instant transition-metal bleach catalyst include, but are not limited to manganese, iron, and chromium. In other embodiments, MRLs include, but are not limited to special ultra-rigid ligands that are cross-bridged (e.g., 5,12-diethyl-1,5,8,12-tetraazabicyclo[6.6.2]hexadecane). Suitable transition metal MRLs are readily prepared by known procedures (See, e.g., WO 2000/32601 and U.S. Pat. No. 6,225,464).

In some embodiments, the cleaning compositions described herein further comprise a metal care agent. Metal care agents are used to prevent and/or reduce tarnishing, corrosion, and/or oxidation of metals, including aluminum, stainless steel, and non-ferrous metals (e.g., silver and copper). Suitable metal care agents include those described in EP2100949, WO94/26860, and WO94/26859). In some embodiments, the metal care agent is a zinc salt. In some further embodiments, the cleaning compositions described herein comprise from about 0.1% to about 5% by weight of one or more metal care agent.

The cleaning compositions described herein can be used to clean a surface, dishware, or fabric. Typically, at least a portion of the surface, dishware, or fabric is contacted with at least one (i) variant described herein, or (ii) at least one cleaning composition described herein, and then the surface, dishware, or fabric is optionally washed and/or rinsed. For purposes of the present disclosure, “washing” includes but is not limited to, scrubbing and mechanical agitation. In some embodiments, the cleaning compositions are typically employed at concentrations of from about 500 ppm to about 15,000 ppm in solution. When the wash solvent is water, the water temperature typically ranges from about 5 °C to about 90 °C and, when fabric is involved, the water to fabric mass ratio is typically from about 1:1 to about 30:1.

Some embodiments are directed to a method of cleaning comprising contacting an effective amount of (i) a mannanase variant described herein, or (ii) a cleaning composition described herein with an item or surface comprising a soil or stain comprising mannan to hydrolyze the mannan contained in the soil or stain.

In some embodiments, one or more mannanase variant described herein is used to prevent, reduce and/or remove a biofilm on one or more item selected from a textile and fabric.

One or more mannanase variant described herein hydrolyzes polysaccharide chains containing mannose units, including, but not limited to, mannans, galactomannans, and glucomannans, making such polypeptides particularly useful for performing mannan hydrolysis reactions involving polysaccharide substrates containing 1,4-β-D-mannosidic linkages. In general terms, a donor molecule is incubated in the presence of a mannanase variant described herein under conditions suitable for performing a mannan hydrolysis reaction, followed by, optionally, isolating a product from the reaction. Alternatively, in the context of a foodstuff, the product may become a component of the foodstuff without isolation. In certain embodiments, the donor molecule is a polysaccharide chain comprising mannose units, including but not limited to mannans, glucomannans, galactomannans, and galactoglucomannans.

In one embodiment, one or more mannanase variants described herein is used in a process for extracting palm kernel oil. Another embodiment is directed to a process for extracting palm kernel oil from palm kernels or a palm kernel meal, comprising providing palm kernels and/or palm kernel meal and treating said seeds or cake with one or more mannanase variant described herein.

In one embodiment, a composition comprising a mannanase variant described herein is used to process and/or manufacture animal feed or food for humans. In yet a further embodiment, a mannanase variant described herein can be an additive to feed for non-human animals. In another embodiment, a mannanase variant described herein can be useful for human food, such as, for example, as an additive to human food.

Several nutritional factors can limit the amount of inexpensive plant material that can be used to prepare animal feed and food for humans. For example, plant material containing oligomannans such as mannan, galactomannan, glucomannan and galactoglucomannan can reduce an animal's ability to digest and absorb nutritional compounds such as minerals, vitamins, sugars, and fats. These negative effects are in particular due to the high viscosity of the mannan-containing polymers and to the ability of the mannan-containing polymers to absorb nutritional compounds. These effects can be reduced by including an enzyme in the feed that degrades the mannan-containing polymers, such as, an endo-β-mannanase enzyme described herein, thereby enabling a higher proportion of mannan-containing polymers typically found in inexpensive plant material to be included in the feed, which ultimately reduces the cost of the feed. Additionally, a mannanase variant described herein can break down the mannan-containing polymers into simpler sugars, which can be more readily assimilated to provide additional energy.

In a further embodiment, animal feed containing plant material is incubated in the presence of a mannanase variant described herein under conditions suitable for breaking down mannan-containing polymers.

In another embodiment, a bread improver composition comprises a mannanase variant described herein, optionally in combination with a source of mannan or glucomannan or galactomannan, and further optionally in combination with one or more other enzymes.

The term “non-human animal” includes all non-ruminant and ruminant animals. In a particular embodiment, the non-ruminant animal is selected from the group consisting of, but is not limited to, horses and monogastric animals such as, but not limited to, pigs, poultry, swine and fish. In further embodiments, the pig may be, but is not limited to, a piglet, a growing pig, and a sow; the poultry may be, but is not limited to, a turkey, a duck and a chicken including, but not limited to, a broiler chick and a layer; and fish may be, but is not limited to salmon, trout, tilapia, catfish and carps; and crustaceans including but not limited to shrimps and prawns. In a further embodiment, the ruminant animal is selected from the group consisting of, but is not limited to, cattle, young calves, goats, sheep, giraffes, bison, moose, elk, yaks, water buffalo, deer, camels, alpacas, llamas, antelope, pronghorn, and nilgai.

In some embodiments, a mannanase variant described herein is used to pretreat feed instead of as a feed additive. In some embodiments, a mannanase variant described herein is added to, or used to pretreat, feed for weanling pigs, nursery pigs, piglets, fattening pigs, growing pigs, finishing pigs, laying hens, broiler chicks, and turkeys.

In another embodiment, a mannanase variant described herein is added to, or used to pretreat, feed from plant material such as palm kernel, coconut, konjac, locust bean gum, gum guar, soy beans, barley, oats, flax, wheat, corn, linseed, citrus pulp, cottonseed, groundnut, rapeseed, sunflower, peas, and lupines.

A mannanase variant described herein is thermostable, and as a result, a mannanase variant described herein can be used in processes of producing pelleted feed in which heat is applied to the feed mixture before the pelleting step. In another embodiment, a mannanase variant described herein is added to the other feed ingredients either in advance of the pelleting step or after the pelleting step (i.e., to the already formed feed pellets).

In yet another embodiment, food processing or feed supplement compositions that contain a mannanase variant described herein may optionally further contain other substituents selected from coloring agents, aroma compounds, stabilizers, vitamins, minerals, and other feed or food enhancing enzymes. This applies in particular to the so-called pre-mixes.

In a still further embodiment, a food additive according to the present invention may be combined in an appropriate amount with other food components, such as, for example, a cereal or plant protein to form a processed food product.

In one embodiment, an animal feed composition and/or animal feed additive composition and/or pet food comprises a polypeptide described herein.

Another embodiment relates to a method for preparing an animal feed composition and/or animal feed additive composition and/or pet food comprising mixing a mannanase variant described herein with one or more animal feed ingredients and/or animal feed additive ingredients and/or pet food ingredients.

A further embodiment relates to the use of a mannanase variant described herein to prepare an animal feed composition and/or animal feed additive composition and/or pet food. The phrase “pet food” means food for a household animal such as, but not limited to, dogs; cats; gerbils; hamsters; chinchillas; fancy rats; guinea pigs; avian pets, such as canaries, parakeets, and parrots; reptile pets, such as turtles, lizards and snakes; and aquatic pets, such as tropical fish and frogs.

The terms animal feed composition, feedstuff and fodder are used interchangeably and may comprise one or more feed materials selected from the group comprising a) cereals, such as small grains (e.g., wheat, barley, rye, oats and combinations thereof) and/or large grains such as maize or sorghum; b) by-products from cereals, such as corn gluten meal, Distillers Dried Grain Solubles (DDGS) (particularly corn based Distillers Dried Grain Solubles (cDDGS)), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, and sesame; d) oils and fats obtained from vegetable and animal sources; and e) minerals and vitamins.

In one aspect, the food composition or additive may be liquid or solid. In an aspect of the invention the food composition is a beverage, including, but not limited to, a fermented beverage such as beer and wine.

In the context of the present invention, the term “fermented beverage” is meant to comprise any beverage produced by a method comprising a fermentation process, such as a microbial fermentation, such as a bacterial and/or yeast fermentation.

In an aspect of the invention the fermented beverage is beer. The term “beer” is meant to comprise any fermented wort produced by fermentation/brewing of a starch-containing plant material. Often, beer is produced from malt or adjunct, or any combination of malt and adjunct as the starch-containing plant material. As used herein the term “malt” is understood as any malted cereal grain, such as malted barley or wheat.

As used herein the term “adjunct” refers to any starch and/or sugar containing plant material which is not malt, such as barley or wheat malt. Examples of adjuncts include, for example, common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, cassava and syrups, such as corn syrup, sugar cane syrup, inverted sugar syrup, barley and/or wheat syrups, and the like may be used as a source of starch.

As used herein, the term “mash” refers to an aqueous slurry of any starch and/or sugar containing plant material such as grist, e. g. comprising crushed barley malt, crushed barley, and/or other adjunct or a combination hereof, mixed with water, later to be separated into wort and spent grains.

As used herein, the term “wort” refers to the unfermented liquor run-off following extracting the grist during mashing.

In another aspect the invention relates to a method of preparing a fermented beverage such as beer comprising mixing a mannanase variant described herein with a malt and/or adjunct.

Examples of beers comprise: full malted beer, beer brewed under the “Reinheitsgebot”, ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, as well as alternative cereal and malt beverages such as fruit flavoured malt beverages, e. g. citrus flavoured, such as lemon-, orange-, lime-, or berry-flavoured malt beverages; liquor flavoured malt beverages, e. g., vodka-, rum-, or tequila-flavoured malt liquor; or coffee flavoured malt beverages, such as caffeine-flavoured malt liquor; and the like.

One aspect of the invention relates to the use of a mannanase variant described herein in the production of a fermented beverage, such as a beer.

Another aspect concerns a method of providing a fermented beverage comprising the step of contacting a mash and/or wort with a mannanase variant described herein.

A further aspect relates to a method of providing a fermented beverage comprising the steps of: (a) preparing a mash, (b) filtering the mash to obtain a wort, and (c) fermenting the wort to obtain a fermented beverage, such as a beer, wherein a mannanase variant described herein is added to: (i) the mash of step (a) and/or (ii) the wort of step (b) and/or (iii) the wort of step (c).

According to yet another aspect, a fermented beverage, such as a beer, is produced or provided by a method comprising the step(s) of (1) contacting a mash and/or a wort with a mannanase variant described herein; and/or (2) (a) preparing a mash, (b) filtering the mash to obtain a wort, and (c) fermenting the wort to obtain a fermented beverage, such as a beer, wherein a mannanase variant described herein is added to: (i) the mash of step (a) and/or (ii) the wort of step (b) and/or (iii) the wort of step (c).

In general terms the coffee extract is incubated in the presence of a mannanase variant described herein under conditions suitable for hydrolyzing galactomannans present in liquid coffee extract.

In another aspect the invention relates to a method of preparing baked products comprising addition of a mannanase variant described herein to dough, followed by baking the dough. Examples of baked products are well known to those skilled in the art and include breads, rolls, puff pastries, sweet fermented doughs, buns, cakes, crackers, cookies, biscuits, waffles, wafers, tortillas, breakfast cereals, extruded products, and the like.

A mannanase variant described herein may be added to dough as part of a bread improver composition. Bread improvers are compositions containing a variety of ingredients, which improve dough properties and the quality of bakery products, e.g. bread and cakes. Bread improvers are often added in industrial bakery processes because of their beneficial effects e.g. the dough stability and the bread texture and volume. Bread improvers usually contain fats and oils as well as additives like emulsifiers, enzymes, antioxidants, oxidants, stabilizers and reducing agents. In addition to any of the polypeptides of the present invention, other enzymes which may also be present in the bread improver or which may be otherwise used in conjunction with any of the polypeptides of the present invention include amylases, hemicellulases, amylolytic complexes, lipases, proteases, xylanases, pectinases, pullulanases, nonstarch polysaccharide degrading enzymes and redox enzymes like glucose oxidase, lipoxygenase or ascorbic acid oxidase.

In one embodiment, a mannanase variant described herein may be added to dough as part of a bread improver composition which also comprises a glucomannan and/or galactomannan source such as konjac gum, guar gum, locust bean gum (Ceratonia siliqua), copra meal, ivory nut mannan (Phytelephas macrocarpa), seaweed mannan extract, coconut meal, and the cell wall of brewer's yeast (may be dried, or used in the form of brewer's yeast extract). Other acceptable mannan derivatives for use in the current invention include unbranched β-1,4-linked mannan homopolymer and manno-oligosaccharides (mannobiose, mannotriose, mannotetraose and mannopentoase). A mannanase variant described herein can be further used either alone, or in combination with a glucomannan and/or galactomannan and/or galactoglucomannan to improve the dough tolerance; dough flexibility and/or dough stickiness; and/or bread crumb structure, as well as retarding staling of the bread. In another aspect, the mannanase hydrolysates act as soluble prebiotics such as manno-oligosaccharides (MOS) which promote the growth of lactic acid bacteria commonly associated with good health when found at favourable population densities in the colon.

In one aspect, the dough to which any polypeptide of the invention is added comprises bran or oat, rice, millet, maize, or legume flour in addition to or instead of pure wheat flour (i.e., is not a pure white flour dough).

In another embodiment, a mannanase variant described herein may be added to milk or any other dairy product to which has also been added a glucomannan and/or galactomannan. Typical glucomannan and/or galactomannan sources are listed above in the bakery aspects, and include guar or konjac gum. The combination of a mannanase variant described herein with a glucomannan and/or galactomannan releases mannanase hydrolysates (mannooligosaccharides) which act as soluble prebiotics by promoting the selective growth and proliferation of probiotic bacteria (especially Bifidobacteria and Lactobacillus lactic acid bacteria) commonly associated with good health when found at favourable population densities in the large intestine or colon.

Another aspect relates to a method of preparing milk or dairy products comprising addition of a mannanase variant described herein and any glucomannan or galactomannan or galactoglucomannan.

In another aspect, a mannanase variant described herein is used in combination with any glucomannan or galactomannan prior to or following addition to a dairy based foodstuff to produce a dairy based foodstuff comprising prebiotic mannan hydrolysates. In a further aspect, the thusly produced mannooligosaccharide-containing dairy product is capable of increasing the population of beneficial human intestinal microflora, and in a yet further aspect the dairy based foodstuff may comprise a mannanase variant described herein together with any source of glucomannan and/or galactomannan and/or galactoglucomannan, and a dose sufficient for inoculation of at least one strain of bacteria (such as Bifidobacteria or Lactobacillus) known to be of benefit in the human large intestine. In one aspect, the dairy-based foodstuff is a yoghurt or milk drink.

The mannanase variant described herein finds further use in the enzyme aided bleaching of paper pulps such as chemical pulps, semi-chemical pulps, kraft pulps, mechanical pulps, and pulps prepared by the sulfite method. In general terms, paper pulps are incubated with a mannanase variant described herein under conditions suitable for bleaching the paper pulp.

In some embodiments, the pulps are chlorine free pulps bleached with oxygen, ozone, peroxide or peroxyacids. In some embodiments, a mannanase variant described herein is used in enzyme aided bleaching of pulps produced by modified or continuous pulping methods that exhibit low lignin contents. In some other embodiments, a mannanase variant described herein is applied alone or preferably in combination with xylanase and/or endoglucanase and/or alpha-galactosidase and/or cellobiohydrolase enzymes.

Galactomannans such as guar gum and locust bean gum are widely used as thickening agents e.g., in food (e.g., ice cream) and print paste for textile printing such as prints on T-shirts. Thus, a mannanase variant described herein also finds use in reducing the thickness or viscosity of mannan-containing substrates. In some embodiments, one or more mannanase variant described herein is used to hydrolyze galactomannans in a food (e.g., ice cream) manufacturing waste stream. In certain embodiments, a mannanase variant described herein is used for reducing the viscosity of residual food in processing equipment thereby facilitating cleaning after processing. In certain other embodiments, a mannanase variant described herein is used for reducing viscosity of print paste, thereby facilitating wash out of surplus print paste after textile printings. In general terms, a mannan-containing substrate is incubated with a mannanase variant described herein under conditions suitable for reducing the viscosity of the mannan-containing substrate.

In yet a further embodiment, one or more mannanase variant described herein can be used in the oil and gas industry to, for example, control the viscosity of drilling fluids; increase the rate at which the fluids used in hydraulic fracturing create subterranean fractures that extend from the borehole into the rock; clean the borehole filter cake; and combinations thereof.

Other aspects and embodiments of the present compositions and methods will be apparent from the foregoing description and following examples. Various alternative embodiments beyond those described herein can be employed in practicing the invention without departing from the spirit and scope of the invention. Accordingly, the claims, and not the specific embodiments described herein, define the scope of the invention and as such methods and structures within the scope of the claims and their equivalents are covered thereby.

Non-limiting examples of compositions and methods disclosed herein are as follows:
1. A mannanase variant comprising an amino acid substitution comparing to a parent mannanase enzyme at a position selected from the group consisting of 32, 72, 161 and 172 wherein the amino acid positions of the variant are numbered by correspondence with the amino acid sequence of the parent enzyme of SEQ ID NO: 1, and wherein the variant has at least 80% identity to the amino acid sequence of SEQ ID NO: 1.
2. A mannanase variant comprising an amino acid substitution, wherein said variant comprises a substitution selected from the group consisting of 19D, 32Y, 34D, 72V, 93Q, 131S, 136P, 139R, 161G, 172F, 225N/Q, 259D, 261D/E and 276W, wherein the amino acid positions of the variant are numbered by correspondence with the amino acid sequence of SEQ ID NO: 1, and wherein the variant has at least 80% identity to the amino acid sequence of SEQ ID NO: 1.
2b A mannanase variant comprising an amino acid substitution, wherein said variant comprises a substitution selected from the group consisting of P019D, F032Y, N034D, 1072V, K093Q, T131S, A136P, D139R, A161G, V172F, G225N/Q, G259D, N261D/E, and F276W, wherein the amino acid positions of the variant are numbered by correspondence with the amino acid sequence of SEQ ID NO: 1, and wherein the variant has at least 80% identity to the amino acid sequence of SEQ ID NO: 1.
2c A mannanase variant comprising an amino acid substitution, wherein said variant comprises a substitution selected from the group consisting of E019D, F032Y, N034D, 1072V, K093Q, T131S, A136P, D139R, A161G, V172F, C225N/Q, G259D, R261D/E, and F276W, wherein the amino acid positions of the variant are numbered by correspondence with the amino acid sequence of SEQ ID NO: 2, and wherein the variant has at least 80% identity to the amino acid sequence of SEQ ID NO: 2.
3. A mannanase variant comprising two or more amino acid substitutions selected from the group consisting of 19D, 32Y, 72V, 93Q, 131S, 136P, 139R, 161G, 168T, 172F, 225N/Q, 259D/E, 261D/E and 276W, wherein the amino acid positions of the variant are numbered by correspondence with the amino acid sequence of SEQ ID NO: 1, and wherein the variant has at least 80% identity to the amino acid sequence of SEQ ID NO: 1.
3a. A mannanase variant comprising two or more amino acid substitutions selected from the group consisting of P019D, F032Y, 1072V, K093Q, T131S, A136P, D139R, A161G, P168T, V172F, G225N/Q, G259D/E, N261D/E and F276W, wherein the amino acid positions of the variant are numbered by correspondence with the amino acid sequence of SEQ ID NO: 1, and wherein the variant has at least 80% identity to the amino acid sequence of SEQ ID NO: 1.
3b. A mannanase variant comprising two or more amino acid substitutions selected from the group consisting of E019D, F032Y, 1072V, K093Q, T131S, A136P, D139R, A161G, P168T, V172F, C225N/Q, G259D/E, R261D/E and F276W, wherein the amino acid positions of the variant are numbered by correspondence with the amino acid sequence of SEQ ID NO: 2, and wherein the variant has at least 80% identity to the amino acid sequence of SEQ ID NO: 2.
4. The mannanase variant of embodiment 3, wherein the variant comprises amino acid substitutions selected from the group consisting of 19D-276W, 32Y-259D, 93Q-276W, 131S-276W, 136P-276W, 139R-276W, 161G-276W, 225N/Q-276W, 259D/E-276W, and 261D/E-276W.
4a. The mannanase variant of embodiment 3b, wherein the variant comprises amino acid substitutions selected from the group consisting of E019D-F276W, F032Y-G259D, K093Q-F276W, T131S-F276W, A136P-F276W, D139R-F276W, A161G-F276W, C225N-F276W, C225Q-F276W, G259D-F276W, G259E-F276W, R261D-F276W and R261E-F276W.
5. The mannanase variant of embodiment 3, wherein the variant comprises amino acid substitutions selected from the group consisting of 19D-131S-276W, 32Y-261D-276W, 32Y-259D-276W, 32Y-172F-259D, 168T-259D-276W, 259D-261E-276W, and 259Q-261E-276W.
5b. The mannanase variant of embodiment 3b, wherein the variant comprises amino acid substitutions selected from the group consisting of E019D-A068S-F276W, E019D-T131S-F276W, F032Y-R261D-F276W, F032Y-G259D-F276W, F032Y-T062E-G259D, F032Y-V172F-G259D, P168A-G259D-F276W, P168T-G259D-F276W, G259D-R261E-F276W, and G259Q-R261E-F276W.
6. A mannanase variant comprising amino acid substitutions selected from the group consisting of Y061W-G259D-R261E-F276W, Y061W-T062E-G259D-R261E-F276W, Y061W-V228T-G259D-R261E-F276W, V228T-G259D-R261E-F276W, F032Y-G259D-R261E-F276W, F032Y-Y061W-Y167F-P168S-G259D-R261E-F276W, F032Y-Y061W-G259D-R261E-F276W, F032Y-Y061W-T062E-G259D-R261E-F276W, F032Y-T062E-R261D-F276W, F032Y-T062E-G259D-F276W, T062E-G259D-R261E-F276W, V059S-Q060L-G259D-R261E-F276W, V059S-Q060L-Y061W-G259D-R261E-F276W, V059S-Q060L-T062E-G259D-R261E-F276W, T062E-V228T-G259D-R261E-F276W, F032Y-T062E-G259D-R261E-F276W, F032Y-T062E-G259D-R261E-F276W-T284E, E019D-A068S-T131S-F276W, F032Y-T062E-G259D-F276W-T284E, F032Y-T062E-V228T-G259D-F276W-T284E, F032Y-T062E-I072V-G259D-F276W-T284E, F032Y-T062E-A068S-G259D-F276W-T284E, F032Y-Y061W-T062E-G259D-F276W-T284E, F032Y-V059S-Q060L-T062E-G259D-F276W-T284E, N010T-F032Y-T062E-G259D-F276W-T284E, F032Y-T062E-Y167F-P168S-V228T-G259D-F276W-T284E, F032Y-T062E-I072V-V228T-G259D-F276W-T284E, F032Y-T062E-I072V-Y167F-P168S-G259D-F276W-T284E, F032Y-T062E-A068S-V228T-G259D-F276W-T284E, F032Y-Y061W-T062E-V228T-G259D-F276W-T284E, F032Y-Y061W-T062E-Y167F-P168S-G259D-F276W-T284E, F032Y-Y061W-T062E-1072V-G259D-F276W-T284E, F032Y-Y061W-T062E-A068S-G259D-F276W-T284E, F032Y-V059S-Q060L-T062E-V228T-G259D-F276W-T284E, F032Y-V059S, Q060L-T062E-Y167F-P168S-G259D-F276W-T284E, F032Y-V059S-Q060L-T062E-1072V-G259D-F276W-T284E, F032Y-V059S-Q060L-T062E-A068S-G259D-F276W-T284E, F032Y-V059S-Q060L-Y061W-T062E-G259D-F276W-T284E, N010T-F032Y-T062E-I072V-G259D-F276W-T284E, N010T-F032Y-T062E-A068S-G259D-F276W-T284E, N010T-F032Y-Y061W-T062E-G259D-F276W-T284E, N010T-F032Y-V059S-Q060L-T062E-G259D-F276W-T284E, F032Y-T062E-I072V-Y167F-P168S-V228T-G259D-F276W-T284E, F032Y-T062E-A068S-Y167F-P168S-V228T-G259D-F276W-T284E, F032Y-T062E-A068S-I072V-Y167F-P168S-G259D-F276W-T284E, F032Y-Y061W-T062E-Y167F-P168S-V228T-G259D-F276W-T284E, F032Y-Y061W-T062E-I072V-V228T-G259D-F276W-T284E, F032Y-Y061W-T062E-I072V-Y167F-P168S-G259D-F276W-T284E, F032Y-Y061W-T062E-A068S-V228T-G259D-F276W-T284E, F032Y-Y061W-T062E-A068S-Y167F-P168S-G259D-F276W-T284E, F032Y-Y061W-T062E-A068S-I072V-G259D-F276W-T284E, F032Y-V059S-Q060L-T062E-Y167F-P168S-V228T-G259D-F276W-T284E, F032Y-V059S-Q060L-T062E-I072V-V228T-G259D-F276W-T284E, F032Y-V059S-Q060L-T062E-1072V-Y167F-P168S-G259D-F276W-T284E, F032Y-V059S-Q060L-T062E-A068S-V228T-G259D-F276W-T284E, F032Y-V059S-Q060L-T062E-A068S-Y167F-P168S-G259D-F276W-T284E, F032Y-V059S-Q060L-T062E-A068S-I072V-G259D-F276W-T284E, F032Y-V059S-Q060L-Y061W-T062E-V228T-G259D-F276W-T284E, F032Y-V059S-Q060L-Y061W-T062E-Y167F-P168S-G259D-F276W-T284E, F032Y-V059S-Q060L-Y061W-T062E-1072V-G259D-F276W-T284E, F032Y-V059S-Q060L-Y061W-T062E-A068S-G259D-F276W-T284E, N010T-F032Y-T062E-I072V-V228T-G259D-F276W-T284E, N010T-F032Y-T062E-A068S-V228T-G259D-F276W-T284E, N010T-F032Y-T062E-A068S-I072V-G259D-F276W-T284E, N010T-F032Y-Y061W-T062E-V228T-G259D-F276W-T284E, N010T-F032Y-Y061W-T062E-Y167F-P168S-G259D-F276W-T284E, N010T-F032Y-Y061W-T062E-I072V-G259D-F276W-T284E, N010T-F032Y-V059S-Q060L-T062E-Y167F-P168S-G259D-F276W-T284E, N010T-F032Y-V059S-Q060L-T062E-I072V-G259D-F276W-T284E, F032Y-T062E-A068S-1072V-Y167F-P168S-V228T-G259D-F276W-T284E, F032Y-Y061W-T062E-1072V-Y167F-P168S-V228T-G259D-F276W-T284E, F032Y-Y061W-T062E-A068S-Y167F-P168S-V228T-G259D-F276W-T284E, F032Y-Y061W-T062E-A068S-I072V-V228T-G259D-F276W-T284E, F032Y-Y061W-T062E-A068S-1072V-Y167F-P168S-G259D-F276W-T284E, F032Y-V059S-Q060L-T062E-1072V-Y167F-P168S-V228T-G259D-F276W-T284E, F032Y-V059S-Q060L-T062E-A068S-Y167F-P168S-V228T-G259D-F276W-T284E, F032Y-V059S-Q060L-T062E-A068S-I072V-V228T-G259D-F276W-T284E, F032Y-V059S-Q060L-T062E-A068S-I072V-Y167F-P168S-G259D-F276W-T284E, F032Y-V059S-Q060L-Y061W-T062E-Y167F-P168S-V228T-G259D-F276W-T284E, F032Y-V059S-Q060L-Y061W-T062E-I072V-V228T-G259D-F276W-T284E, F032Y-V059S-Q060L-Y061W-T062E-1072V-Y167F-P168S-G259D-F276W-T284E, F032Y-V059S-Q060L-Y061W-T062E-A068S-V228T-G259D-F276W-T284E, F032Y-V059S-Q060L-Y061W-T062E-A068S-Y167F-P168S-G259D-F276W-T284E, F032Y-V059S-Q060L-Y061W-T062E-A068S-I072V-G259D-F276W-T284E, N010T-F032Y-T062E-I072V-Y167F-P168S-V228T-G259D-F276W-T284E, N010T-F032Y-T062E-A068S-Y167F-P168S-V228T-G259D-F276W-T284E, N010T-F032Y-T062E-A068S-I072V-V228T-G259D-F276W-T284E, N010T-F032Y-T062E-A068S-I072V-Y167F-P168S-G259D-F276W-T284E, N010T-F032Y-Y061W-T062E-Y167F-P168S-V228T-G259D-F276W-T284E, N010T-F032Y-Y061W-T062E-I072V-V228T-G259D-F276W-T284E, N010T-F032Y-Y061W-T062E-I072V-Y167F-P168S-G259D-F276W-T284E, N010T-F032Y-Y061W-T062E-A068S-Y167F-P168S-G259D-F276W-T284E, N010T-F032Y-Y061W-T062E-A068S-I072V-G259D-F276W-T284E, N010T-F032Y-V059S-Q060L-T062E-Y167F-P168S-V228T-G259D-F276W-T284E, N010T-F032Y-V059S-Q060L-T062E-I072V-V228T-G259D-F276W-T284E, N010T-F032Y-V059S-Q060L-T062E-1072V-Y167F-P168S-G259D-F276W-T284E, N010T-F032Y-V059S-Q060L-T062E-A068S-V228T-G259D-F276W-T284E, N010T-F032Y-V059S-Q060L-T062E-A068S-1072V-G259D-F276W-T284E, N010T-F032Y-V059S-Q060L-Y061W-T062E-V228T-G259D-F276W-T284E, N010T-F032Y-V059S-Q060L-Y061W-T062E-Y167F-P168S-G259D-F276W-T284E, N010T-F032Y-V059S-Q060L-Y061W-T062E-A068S-G259D-F276W-T284E, F032Y-Y061W-T062E-A068S-I072V-Y167F-P168S-V228T-G259D-F276W-T284E, F032Y-V059S-Q060L-T062E-A068S-1072V-Y167F-P168S-V228T-G259D-F276W-T284E, F032Y-V059S-Q060L-Y061W-T062E-1072V-Y167F-P168S-V228T-G259D-F276W-T284E, F032Y-V059S-Q060L-Y061W-T062E-A068S-Y167F-P168S-V228T-G259D-F276W-T284E, F032Y-V059S-Q060L-Y061W-T062E-A068S-1072V-V228T-G259D-F276W-T284E, F032Y-V059S-Q060L-Y061W-T062E-A068S-I072V-Y167F-P168S-G259D-F276W-T284E, N010T-F032Y-T062E-A068S-I072V-Y167F-P168S-V228T-G259D-F276W-T284E, N010T-F032Y-Y061W-T062E-I072V-Y167F-P168S-V228T-G259D-F276W-T284E, N010T-F032Y-Y061W-T062E-A068S-Y167F-P168S-V228T-G259D-F276W-T284E, N010T-F032Y-Y061W-T062E-A068S-I072V-V228T-G259D-F276W-T284E, N010T-F032Y-V059S-Q060L-T062E-1072V-Y167F-P168S-V228T-G259D-F276W-T284E, N010T-F032Y-V059S-Q060L-T062E-A068S-Y167F-P168S-V228T-G259D-F276W-T284E, N010T-F032Y-V059S-Q060L-T062E-A068S-1072V-V228T-G259D-F276W-T284E, N010T-F032Y-V059S-Q060L-T062E-A068S-1072V-Y167F-P168S-G259D-F276W-T284E, N010T-F032Y-V059S-Q060L-Y061W-T062E-Y167F-P168S-V228T-G259D-F276W-T284E, N010T-F032Y-V059S-Q060L-Y061W-T062E-1072V-Y167F-P168S-G259D-F276W-T284E, N010T-F032Y-V059S-Q060L-Y061W-T062E-A068S-Y167F-P168S-G259D-F276W-T284E, N010T-F032Y-V059S-Q060L-Y061W-T062E-A068S-1072V-G259D-F276W-T284E, N010T-F032Y-Y061W-T062E-A068S-I072V-Y167F-P168S-V228T-G259D-F276W-T284E, N010T-F032Y-V059S-Q060L-T062E-A068S-1072V-Y167F-P168S-V228T-G259D-F276W-T284E, N010T-F032Y-V059S-Q060L-Y061W-T062E-I072V-Y167F-P168S-V228T-G259D-F276W-T284E, N010T-F032Y-V059S-Q060L-Y061W-T062E-A068S-Y167F-P168S-V228T-G259D-F276W-T284E, N010T-F032Y-V059S-Q060L-Y061W-T062E-A068S-I072V-V228T-G259D-F276W-T284E, N010T-F032Y-V059S-Q060L-Y061W-T062E-A068S-I072V-Y167F-P168S-G259D-F276W-T284E and N010T-F032Y-V059S-Q060L-Y061W-T062E-A068S-I072V-Y167F-P168S-V228T-G259D-F276W-T284E, F032Y-Y061W-T062E-G259D-R261E-F276W-T284E, F032Y-T062E-A068S-G259D-R261E-F276W-T284E, F032Y-T062E-T131S-G259D-R261E-F276W-T284E, F032Y-T062E-N150D-G259D-R261E-F276W-T284E, F032Y-T062E-P168S-G259D-R261E-F276W-T284E, F032Y-T062E-L235K-G259D-R261E-F276W-T284E, N010T-F032Y-Y061W-T062E-P168A-V228T-G259D-F276W-T284E, N010T-F032Y-Y061W-T062E-Y167F-P168S-V228T-G259E-F276W-T284E, F032Y-Y061W-T062E-T131S-Y167F-P168S-V228T-G259D-F276W-T284E, N010T-F032Y-Y061W-T062E-A136P-Y167F-P168S-V228T-G259D-F276W-T284E, N010T-F032Y-Y061W-T062E-K093Q-Y167F-P168S-V228T-G259D-F276W-T284E, N010T-F032Y-Y061W-T062E-Y167F-P168S-V228T-L235K-G259D-F276W-T284E, N010T-F032Y-Y061W-T062E-D139R-Y167F-P168S-V228T-G259D-F276W-T284E, N010T-F032Y-Y061W-T062E-N150D-Y167F-P168S-V228T-G259D-F276W-T284E, N010T-F032Y-Y061W-T062E-Y167F-P168A-V228T-G259D-F276W-T284E, N010T-F032Y-Y061W-T062E-T131S-Y167F-P168S-V228T-G259D-F276W-T284E, N010T-F032Y-Y061W-T062E-P168S-V228T-G259D-F276W-T284E, N010T-F032Y-Y061W-T062E-A068S-Y167F-P168A-G259D-F276W-T284E, and N010T-F032Y-Y061W-T062E-A068S-Y167F-P168A-G259E-F276W-T284E, wherein the amino acid positions of the variant are numbered by correspondence with the amino acid sequence of SEQ ID NO: 1, and wherein the variant has at least 80% identity to the amino acid sequence of SEQ ID NO: 1.
7. The mannanase variant of embodiments 1-3, wherein the variant comprises an amino acid sequence having at least 85%, 90%, 91%, 92%, 93%, 94%, 95%, 96%, 97%, 98%, or 99% sequence identity to the amino acid sequence of SEQ ID NO: 1.
7a. The mannanase variant according to any preceding embodiment, wherein the variant comprises an amino acid sequence having at least 85%, 90%, 91%, 92%, 93%, 94%, 95%, 96%, 97%, 98%, or 99% sequence identity to the amino acid sequence of SEQ ID NO: 1.
8. The mannanase variant according to embodiments 1-3, wherein said variant is derived from a parent or reference polypeptide with 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1.
8a. The mannanase variant according to any preceding embodiment, wherein said variant is derived from a parent or reference polypeptide with 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1 or SEQ ID NO: 2 or SEQ ID NO:3 or SEQ ID NO: 4 or SEQ ID NO:5 or SEQ ID NO: 6.
9. The mannanase variant of embodiments 1-3, wherein said variant has improved stability when compared to a parent or reference mannanase.
9a. The mannanase variant according to any preceding embodiment, wherein said variant has improved stability when compared to a parent or reference mannanase.
10. The mannanase of embodiment 9, wherein said variant has equal or improved cleaning performance in a detergent when compared to a parent or reference mannanase.
10. The mannanase of embodiment 9, wherein said variant has equal or improved cleaning performance in a detergent when compared to a parent or reference mannanase.
10b. The mannanase according to any preceding embodiment, wherein said variant has equal or improved cleaning performance in a detergent when compared to a parent or reference mannanase
11. The mannanase variant of embodiments 1-3, wherein the mannanase variant has mannanase activity.
11a. The mannanase variant according to any preceding embodiment, wherein the mannanase variant has mannanase activity.
11b. The mannanase variant of any preceding embodiment, wherein the mannanase activity is in the presence of a surfactant and/or a protease.
12. A polynucleotide comprising a nucleic acid sequence encoding a variant of any one of embodiments 1-3, wherein said polynucleotide is, optionally, isolated.
12a. A polynucleotide comprising a nucleic acid sequence encoding a variant according to any preceding embodiment, wherein said polynucleotide is, optionally, isolated.
12b. The polynucleotide of embodiment 12 or 12a, wherein the nucleic acid sequence is operably linked to a promoter.
12c. An expression vector or cassette comprising the polynucleotide of embodiment 12 or 12a or 12b.
12d. A recombinant host cell comprising the polynucleotide of embodiment 12 or 12a or 12b or the vector or cassette of Embodiment 12d.
13. An enzyme composition comprising one or more mannanase variant according to embodiments 1-3.
13a. An enzyme composition comprising one or more mannanase variant according to any preceding embodiment.
14. The enzyme composition according to embodiment 13 or 13a, wherein said composition is an enzyme granule.
15. The enzyme composition according to any one of embodiments 13, 13a, or 14, further comprising one or more other enzymes selected from acyl transferases, amylases, alpha-amylases, beta-amylases, alpha-galactosidases, arabinases, arabinosidases, aryl esterases, beta-galactosidases, beta-glucanases, carrageenases, catalases, cellulases, chondroitinases, cutinases, dispersins, DNAses (also known as nucleases or dispersins), endo-glucanases, endo-beta-mannanases, exo-beta-mannanases, esterases, exo-mannanases, galactanases, glucoamylases, hemicellulases, hexosaminidase, hyaluronidases, keratinases, laccases, lactases, ligninases, lipases, lipolytic enzymes, lipoxygenases, lysozyme, mannanases, metalloproteases, nucleases, oxidases, oxidoreductases, pectate lyases, pectin acetyl esterases, pectinases, pentosanases, perhydrolases, peroxidases, PETases, phenoloxidases, phosphatases, phospholipases, phosphodiesterases, phytases, polyesterases, polygalacturonases, proteases, pullulanases, reductases, rhamnogalacturonases, tannases, transglutaminases, Xanthan lyases, Xanthanases, xylan acetyl-esterases, xylanases, xylosidases, and combinations thereof or mixture thereof.
16. A cleaning composition comprising the mannanase variant of any one of embodiments 1-3.
16a. A cleaning composition comprising the mannanase variant of any one of embodiments 1-11.
16b. The cleaning composition of embodiment 16, further comprising: at least one surfactant; at least one ion selected from calcium and zinc; at least one adjunct ingredient; at least one stabilizer; from about 0.001% to about 1.0 weight % of said mannanase variant of any one of embodiments 1-11; at least one bleaching agent; and/or at least one enzyme or enzyme derivative selected from the group consisting of acyl transferases, amylases, alpha-amylases, beta-amylases, alpha-galactosidases, arabinases, arabinosidases, aryl esterases, beta-galactosidases, beta-glucanases, carrageenases, catalases, cellulases, chondroitinases, cutinases, dispersins, DNAses (also known as nucleases or dispersins), endo-glucanases, endo-beta-mannanases, exo-beta-mannanases, esterases, exo-mannanases, galactanases, glucoamylases, hemicellulases, hexosaminidase, hyaluronidases, keratinases, laccases, lactases, ligninases, lipases, lipolytic enzymes, lipoxygenases, lysozyme, mannanases, metalloproteases, nucleases, oxidases, oxidoreductases, pectate lyases, pectin acetyl esterases, pectinases, pentosanases, perhydrolases, peroxidases, PETases, phenoloxidases, phosphatases, phospholipases, phosphodiesterases, phytases, polyesterases, polygalacturonases, proteases, pullulanases, reductases, rhamnogalacturonases, tannases, transglutaminases, Xanthan lyases, Xanthanases, xylan acetyl-esterases, xylanases, xylosidases, and combinations thereof or mixture thereof.
17. The cleaning composition of embodiment 16, wherein the cleaning composition is a detergent composition selected from the group consisting of a laundry detergent, a fabric softening detergent, a dishwashing detergent, a medical instrument cleaning detergent, and a hard-surface cleaning detergent.
17a. The cleaning composition of any one of embodiments 16-17, wherein the cleaning composition is in a form selected from a liquid, a powder, a granulated solid, a tablet, a sheet, and a unit dose.
17b. The cleaning composition of any one of embodiments 16-17 or embodiment 17a, wherein said composition contains phosphate or is phosphate-free and/or contains boron or is boron-free.
18. A method of cleaning comprising, contacting a surface or an item in need of cleaning with an effective amount of a mannanase variant of any one of embodiments 1-3 or the enzyme composition of any one of embodiments 13-15; and optionally further comprising the step of rinsing said surface or item after contacting said surface or item with said variant or enzyme composition.
19. The method of embodiment 18, wherein said item is a medical instrument, dishware or fabric.
20. A method for producing a mannanase variant of any one of embodiments 1-3 comprising: (a) stably transforming a host cell of embodiment 12c with the expression vector of embodiment 12b; (b) cultivating said transformed host cell under conditions suitable for said host cell to produce said mannanase variant or recombinant polypeptide or active fragment thereof; and (c) recovering said mannanase variant or recombinant polypeptide or active fragment thereof.
21. A food or feed composition and/or food additive comprising the mannanase variant or recombinant polypeptide of any one of embodiments 1-3.
22. Use of the mannanase variant or recombinant polypeptide of any one of embodiments 1-3 in the preparation of a food or feed composition and/or food or feed additive and/or food or feed stuff and/or pet food
23. A mannanase variant comprising amino acid substitutions selected from the group consisting of X0136P-X019D, X0136P-X0225N, X0136P-X032Y, X0136P-X072V, X0136P-X093Q, X0136P-X131S, X0136P-X139R, X0136P-X161G, X0136P-X168T, X0136P-X172F, X0136P-X225Q, X0136P-X259D, X0136P-X259E, X0136P-X261D, X0136P-X261E, X0136P-X276W, X019D-X0225N, X019D-X032Y, X019D-X072V, X019D-X093Q, X019D-X131S, X019D-X139R, X019D-X161G, X019D-X168T, X019D-X172F, X019D-X225Q, X019D-X259D, X019D-X259E, X019D-X261D, X019D-X261E, X019D-X276W, X0225N-X032Y, X0225N-X072V, X0225N-X093Q, X0225N-X131S, X0225N-X139R, X0225N-X161G, X0225N-X168T, X0225N-X172F, X0225N-X259D, X0225N-X259E, X0225N-X261D, X0225N-X261E, X0225N-X276W, X032Y-X072V, X032Y-X093Q, X032Y-X131S, X032Y-X139R, X032Y-X161G, X032Y-X168T, X032Y-X172F, X032Y-X225Q, X032Y-X259D, X032Y-X259E, X032Y-X261D, X032Y-X261E, X032Y-X276W, X072V-X093Q, X072V-X131S, X072V-X139R, X072V-X161G, X072V-X168T, X072V-X172F, X072V-X225Q, X072V-X259D, X072V-X259E, X072V-X261D, X072V-X261E, X072V-X276W, X093Q-X131S, X093Q-X139R, X093Q-X161G, X093Q-X168T, X093Q-X172F, X093Q-X225Q, X093Q-X259D, X093Q-X259E, X093Q-X261D, X093Q-X261E, X093Q-X276W, X131S-X139R, X131S-X161G, X131S-X168T, X131S-X172F, X131S-X225Q, X131S-X259D, X131S-X259E, X131S-X261D, X131S-X261E, X131S-X276W, X139R-X161G, X139R-X168T, X139R-X172F, X139R-X225Q, X139R-X259D, X139R-X259E, X139R-X261D, X139R-X261E, X139R-X276W, X161G-X168T, X161G-X172F, X161G-X225Q, X161G-X259D, X161G-X259E, X161G-X261D, X161G-X261E, X161G-X276W, X168T-X172F, X168T-X225Q, X168T-X259D, X168T-X259E, X168T-X261D, X168T-X261E, X168T-X276W, X172F-X225Q, X172F-X259D, X172F-X259E, X172F-X261D, X172F-X261E, X172F-X276W, X225Q-X259D, X225Q-X259E, X225Q-X261D, X225Q-X261E, X225Q-X276W, X259D-X261D, X259D-X261E, X259D-X276W, X259E-X261D, X259E-X261E, X259E-X276W, X261D-X276W and X261E-X276W.
24. A mannanase variant comprising amino acid substitutions selected from the group consisting of X0136P-X019D-X0225N, X0136P-X019D-X032Y, X0136P-X019D-X072V, X0136P-X019D-X093Q, X0136P-X019D-X131S, X0136P-X019D-X139R, X0136P-X019D-X161G, X0136P-X019D-X168T, X0136P-X019D-X172F, X0136P-X019D-X225Q, X0136P-X019D-X259D, X0136P-X019D-X259E, X0136P-X019D-X261D, X0136P-X019D-X261E, X0136P-X019D-X276W, X0136P-X0225N-X032Y, X0136P-X0225N-X072V, X0136P-X0225N-X093Q, X0136P-X0225N-X131S, X0136P-X0225N-X139R, X0136P-X0225N-X161G, X0136P-X0225N-X168T, X0136P-X0225N-X172F, X0136P-X0225N-X259D, X0136P-X0225N-X259E, X0136P-X0225N-X261D, X0136P-X0225N-X261E, X0136P-X0225N-X276W, X0136P-X032Y-X072V, X0136P-X032Y-X093Q, X0136P-X032Y-X131S, X0136P-X032Y-X139R, X0136P-X032Y-X161G, X0136P-X032Y-X168T, X0136P-X032Y-X172F, X0136P-X032Y-X225Q, X0136P-X032Y-X259D, X0136P-X032Y-X259E, X0136P-X032Y-X261D, X0136P-X032Y-X261E, X0136P-X032Y-X276W, X0136P-X072V-X093Q, X0136P-X072V-X131S, X0136P-X072V-X139R, X0136P-X072V-X161G, X0136P-X072V-X168T, X0136P-X072V-X172F, X0136P-X072V-X225Q, X0136P-X072V-X259D, X0136P-X072V-X259E, X0136P-X072V-X261D, X0136P-X072V-X261E, X0136P-X072V-X276W, X0136P-X093Q-X131S, X0136P-X093Q-X139R, X0136P-X093Q-X161G, X0136P-X093Q-X168T, X0136P-X093Q-X172F, X0136P-X093Q-X225Q, X0136P-X093Q-X259D, X0136P-X093Q-X259E, X0136P-X093Q-X261D, X0136P-X093Q-X261E, X0136P-X093Q-X276W, X0136P-X131S-X139R, X0136P-X131S-X161G, X0136P-X131S-X168T, X0136P-X131S-X172F, X0136P-X131S-X225Q, X0136P-X131S-X259D, X0136P-X131S-X259E, X0136P-X131S-X261D, X0136P-X131S-X261E, X0136P-X131S-X276W, X0136P-X139R-X161G, X0136P-X139R-X168T, X0136P-X139R-X172F, X0136P-X139R-X225Q, X0136P-X139R-X259D, X0136P-X139R-X259E, X0136P-X139R-X261D, X0136P-X139R-X261E, X0136P-X139R-X276W, X0136P-X161G-X168T, X0136P-X161G-X172F, X0136P-X161G-X225Q, X0136P-X161G-X259D, X0136P-X161G-X259E, X0136P-X161G-X261D, X0136P-X161G-X261E, X0136P-X161G-X276W, X0136P-X168T-X172F, X0136P-X168T-X225Q, X0136P-X168T-X259D, X0136P-X168T-X259E, X0136P-X168T-X261D, X0136P-X168T-X261E, X0136P-X168T-X276W, X0136P-X172F-X225Q, X0136P-X172F-X259D, X0136P-X172F-X259E, X0136P-X172F-X261D, X0136P-X172F-X261E, X0136P-X172F-X276W, X0136P-X225Q-X259D, X0136P-X225Q-X259E, X0136P-X225Q-X261D, X0136P-X225Q-X261E, X0136P-X225Q-X276W, X0136P-X259D-X261D, X0136P-X259D-X261E, X0136P-X259D-X276W, X0136P-X259E-X261D, X0136P-X259E-X261E, X0136P-X259E-X276W, X0136P-X261D-X276W, X0136P-X261E-X276W, X019D-X0225N-X032Y, X019D-X0225N-X072V, X019D-X0225N-X093Q, X019D-X0225N-X131S, X019D-X0225N-X139R, X019D-X0225N-X161G, X019D-X0225N-X168T, X019D-X0225N-X172F, X019D-X0225N-X259D, X019D-X0225N-X259E, X019D-X0225N-X261D, X019D-X0225N-X261E, X019D-X0225N-X276W, X019D-X032Y-X072V, X019D-X032Y-X093Q, X019D-X032Y-X131S, X019D-X032Y-X139R, X019D-X032Y-X161G, X019D-X032Y-X168T, X019D-X032Y-X172F, X019D-X032Y-X225Q, X019D-X032Y-X259D, X019D-X032Y-X259E, X019D-X032Y-X261D, X019D-X032Y-X261E, X019D-X032Y-X276W, X019D-X072V-X093Q, X019D-X072V-X131S, X019D-X072V-X139R, X019D-X072V-X161G, X019D-X072V-X168T, X019D-X072V-X172F, X019D-X072V-X225Q, X019D-X072V-X259D, X019D-X072V-X259E, X019D-X072V-X261D, X019D-X072V-X261E, X019D-X072V-X276W, X019D-X093Q-X131S, X019D-X093Q-X139R, X019D-X093Q-X161G, X019D-X093Q-X168T, X019D-X093Q-X172F, X019D-X093Q-X225Q, X019D-X093Q-X259D, X019D-X093Q-X259E, X019D-X093Q-X261D, X019D-X093Q-X261E, X019D-X093Q-X276W, X019D-X131S-X139R, X019D-X131S-X161G, X019D-X131S-X168T, X019D-X131S-X172F, X019D-X131S-X225Q, X019D-X131S-X259D, X019D-X131S-X259E, X019D-X131S-X261D, X019D-X131S-X261E, X019D-X131S-X276W, X019D-X139R-X161G, X019D-X139R-X168T, X019D-X139R-X172F, X019D-X139R-X225Q, X019D-X139R-X259D, X019D-X139R-X259E, X019D-X139R-X261D, X019D-X139R-X261E, X019D-X139R-X276W, X019D-X161G-X168T, X019D-X161G-X172F, X019D-X161G-X225Q, X019D-X161G-X259D, X019D-X161G-X259E, X019D-X161G-X261D, X019D-X161G-X261E, X019D-X161G-X276W, X019D-X168T-X172F, X019D-X168T-X225Q, X019D-X168T-X259D, X019D-X168T-X259E, X019D-X168T-X261D, X019D-X168T-X261E, X019D-X168T-X276W, X019D-X172F-X225Q, X019D-X172F-X259D, X019D-X172F-X259E, X019D-X172F-X261D, X019D-X172F-X261E, X019D-X172F-X276W, X019D-X225Q-X259D, X019D-X225Q-X259E, X019D-X225Q-X261D, X019D-X225Q-X261E, X019D-X225Q-X276W, X019D-X259D-X261D, X019D-X259D-X261E, X019D-X259D-X276W, X019D-X259E-X261D, X019D-X259E-X261E, X019D-X259E-X276W, X019D-X261D-X276W, X019D-X261E-X276W, X0225N-X032Y-X072V, X0225N-X032Y-X093Q, X0225N-X032Y-X131S, X0225N-X032Y-X139R, X0225N-X032Y-X161G, X0225N-X032Y-X168T, X0225N-X032Y-X172F, X0225N-X032Y-X259D, X0225N-X032Y-X259E, X0225N-X032Y-X261D, X0225N-X032Y-X261E, X0225N-X032Y-X276W, X0225N-X072V-X093Q, X0225N-X072V-X131S, X0225N-X072V-X139R, X0225N-X072V-X161G, X0225N-X072V-X168T, X0225N-X072V-X172F, X0225N-X072V-X259D, X0225N-X072V-X259E, X0225N-X072V-X261D, X0225N-X072V-X261E, X0225N-X072V-X276W, X0225N-X093Q-X131S, X0225N-X093Q-X139R, X0225N-X093Q-X161G, X0225N-X093Q-X168T, X0225N-X093Q-X172F, X0225N-X093Q-X259D, X0225N-X093Q-X259E, X0225N-X093Q-X261D, X0225N-X093Q-X261E, X0225N-X093Q-X276W, X0225N-X131S-X139R, X0225N-X131S-X161G, X0225N-X131S-X168T, X0225N-X131S-X172F, X0225N-X131S-X259D, X0225N-X131S-X259E, X0225N-X131S-X261D, X0225N-X131S-X261E, X0225N-X131S-X276W, X0225N-X139R-X161G, X0225N-X139R-X168T, X0225N-X139R-X172F, X0225N-X139R-X259D, X0225N-X139R-X259E, X0225N-X139R-X261D, X0225N-X139R-X261E, X0225N-X139R-X276W, X0225N-X161G-X168T, X0225N-X161G-X172F, X0225N-X161G-X259D, X0225N-X161G-X259E, X0225N-X161G-X261D, X0225N-X161G-X261E, X0225N-X161G-X276W, X0225N-X168T-X172F, X0225N-X168T-X259D, X0225N-X168T-X259E, X0225N-X168T-X261D, X0225N-X168T-X261E, X0225N-X168T-X276W, X0225N-X172F-X259D, X0225N-X172F-X259E, X0225N-X172F-X261D, X0225N-X172F-X261E, X0225N-X172F-X276W, X0225N-X259D-X261D, X0225N-X259D-X261E, X0225N-X259D-X276W, X0225N-X259E-X261D, X0225N-X259E-X261E, X0225N-X259E-X276W, X0225N-X261D-X276W, X0225N-X261E-X276W, X032Y-X072V-X093Q, X032Y-X072V-X131S, X032Y-X072V-X139R, X032Y-X072V-X161G, X032Y-X072V-X168T, X032Y-X072V-X172F, X032Y-X072V-X225Q, X032Y-X072V-X259D, X032Y-X072V-X259E, X032Y-X072V-X261D, X032Y-X072V-X261E, X032Y-X072V-X276W, X032Y-X093Q-X131S, X032Y-X093Q-X139R, X032Y-X093Q-X161G, X032Y-X093Q-X168T, X032Y-X093Q-X172F, X032Y-X093Q-X225Q, X032Y-X093Q-X259D, X032Y-X093Q-X259E, X032Y-X093Q-X261D, X032Y-X093Q-X261E, X032Y-X093Q-X276W, X032Y-X131S-X139R, X032Y-X131S-X161G, X032Y-X131S-X168T, X032Y-X131S-X172F, X032Y-X131S-X225Q, X032Y-X131S-X259D, X032Y-X131S-X259E, X032Y-X131S-X261D, X032Y-X131S-X261E, X032Y-X131S-X276W, X032Y-X139R-X161G, X032Y-X139R-X168T, X032Y-X139R-X172F, X032Y-X139R-X225Q, X032Y-X139R-X259D, X032Y-X139R-X259E, X032Y-X139R-X261D, X032Y-X139R-X261E, X032Y-X139R-X276W, X032Y-X161G-X168T, X032Y-X161G-X172F, X032Y-X161G-X225Q, X032Y-X161G-X259D, X032Y-X161G-X259E, X032Y-X161G-X261D, X032Y-X161G-X261E, X032Y-X161G-X276W, X032Y-X168T-X172F, X032Y-X168T-X225Q, X032Y-X168T-X259D, X032Y-X168T-X259E, X032Y-X168T-X261D, X032Y-X168T-X261E, X032Y-X168T-X276W, X032Y-X172F-X225Q, X032Y-X172F-X259D, X032Y-X172F-X259E, X032Y-X172F-X261D, X032Y-X172F-X261E, X032Y-X172F-X276W, X032Y-X225Q-X259D, X032Y-X225Q-X259E, X032Y-X225Q-X261D, X032Y-X225Q-X261E, X032Y-X225Q-X276W, X032Y-X259D-X261D, X032Y-X259D-X261E, X032Y-X259D-X276W, X032Y-X259E-X261D, X032Y-X259E-X261E, X032Y-X259E-X276W, X032Y-X261D-X276W, X032Y-X261E-X276W, X072V-X093Q-X131S, X072V-X093Q-X139R, X072V-X093Q-X161G, X072V-X093Q-X168T, X072V-X093Q-X172F, X072V-X093Q-X225Q, X072V-X093Q-X259D, X072V-X093Q-X259E, X072V-X093Q-X261D, X072V-X093Q-X261E, X072V-X093Q-X276W, X072V-X131S-X139R, X072V-X131S-X161G, X072V-X131S-X168T, X072V-X131S-X172F, X072V-X131S-X225Q, X072V-X131S-X259D, X072V-X131S-X259E, X072V-X131S-X261D, X072V-X131S-X261E, X072V-X131S-X276W, X072V-X139R-X161G, X072V-X139R-X168T, X072V-X139R-X172F, X072V-X139R-X225Q, X072V-X139R-X259D, X072V-X139R-X259E, X072V-X139R-X261D, X072V-X139R-X261E, X072V-X139R-X276W, X072V-X161G-X168T, X072V-X161G-X172F, X072V-X161G-X225Q, X072V-X161G-X259D, X072V-X161G-X259E, X072V-X161G-X261D, X072V-X161G-X261E, X072V-X161G-X276W, X072V-X168T-X172F, X072V-X168T-X225Q, X072V-X168T-X259D, X072V-X168T-X259E, X072V-X168T-X261D, X072V-X168T-X261E, X072V-X168T-X276W, X072V-X172F-X225Q, X072V-X172F-X259D, X072V-X172F-X259E, X072V-X172F-X261D, X072V-X172F-X261E, X072V-X172F-X276W, X072V-X225Q-X259D, X072V-X225Q-X259E, X072V-X225Q-X261D, X072V-X225Q-X261E, X072V-X225Q-X276W, X072V-X259D-X261D, X072V-X259D-X261E, X072V-X259D-X276W, X072V-X259E-X261D, X072V-X259E-X261E, X072V-X259E-X276W, X072V-X261D-X276W, X072V-X261E-X276W, X093Q-X131S-X139R, X093Q-X131S-X161G, X093Q-X131S-X168T, X093Q-X131S-X172F, X093Q-X131S-X225Q, X093Q-X131S-X259D, X093Q-X131S-X259E, X093Q-X131S-X261D, X093Q-X131S-X261E, X093Q-X131S-X276W, X093Q-X139R-X161G, X093Q-X139R-X168T, X093Q-X139R-X172F, X093Q-X139R-X225Q, X093Q-X139R-X259D, X093Q-X139R-X259E, X093Q-X139R-X261D, X093Q-X139R-X261E, X093Q-X139R-X276W, X093Q-X161G-X168T, X093Q-X161G-X172F, X093Q-X161G-X225Q, X093Q-X161G-X259D, X093Q-X161G-X259E, X093Q-X161G-X261D, X093Q-X161G-X261E, X093Q-X161G-X276W, X093Q-X168T-X172F, X093Q-X168T-X225Q, X093Q-X168T-X259D, X093Q-X168T-X259E, X093Q-X168T-X261D, X093Q-X168T-X261E, X093Q-X168T-X276W, X093Q-X172F-X225Q, X093Q-X172F-X259D, X093Q-X172F-X259E, X093Q-X172F-X261D, X093Q-X172F-X261E, X093Q-X172F-X276W, X093Q-X225Q-X259D, X093Q-X225Q-X259E, X093Q-X225Q-X261D, X093Q-X225Q-X261E, X093Q-X225Q-X276W, X093Q-X259D-X259E, X093Q-X259D-X261D, X093Q-X259D-X261E, X093Q-X259D-X276W, X093Q-X259E-X261D, X093Q-X259E-X261E, X093Q-X259E-X276W, X093Q-X261D-X276W, X093Q-X261E-X276W, X131S-X139R-X161G, X131S-X139R-X168T, X131S-X139R-X172F, X131S-X139R-X225Q, X131S-X139R-X259D, X131S-X139R-X259E, X131S-X139R-X261D, X131S-X139R-X261E, X131S-X139R-X276W, X131S-X161G-X168T, X131S-X161G-X172F, X131S-X161G-X225Q, X131S-X161G-X259D, X131S-X161G-X259E, X131S-X161G-X261D, X131S-X161G-X261E, X131S-X161G-X276W, X131S-X168T-X172F, X131S-X168T-X225Q, X131S-X168T-X259D, X131S-X168T-X259E, X131S-X168T-X261D, X131S-X168T-X261E, X131S-X168T-X276W, X131S-X172F-X225Q, X131S-X172F-X259D, X131S-X172F-X259E, X131S-X172F-X261D, X131S-X172F-X261E, X131S-X172F-X276W, X131S-X225Q-X259D, X131S-X225Q-X259E, X131S-X225Q-X261D, X131S-X225Q-X261E, X131S-X225Q-X276W, X131S-X259D-X261D, X131S-X259D-X261E, X131S-X259D-X276W, X131S-X259E-X261D, X131S-X259E-X261E, X131S-X259E-X276W, X131S-X261D-X276W, X131S-X261E-X276W, X139R-X161G-X168T, X139R-X161G-X172F, X139R-X161G-X225Q, X139R-X161G-X259D, X139R-X161G-X259E, X139R-X161G-X261D, X139R-X161G-X261E, X139R-X161G-X276W, X139R-X168T-X172F, X139R-X168T-X225Q, X139R-X168T-X259D, X139R-X168T-X259E, X139R-X168T-X261D, X139R-X168T-X261E, X139R-X168T-X276W, X139R-X172F-X225Q, X139R-X172F-X259D, X139R-X172F-X259E, X139R-X172F-X261D, X139R-X172F-X261E, X139R-X172F-X276W, X139R-X225Q-X259D, X139R-X225Q-X259E, X139R-X225Q-X261D, X139R-X225Q-X261E, X139R-X225Q-X276W, X139R-X259D-X261D, X139R-X259D-X261E, X139R-X259D-X276W, X139R-X259E-X261D, X139R-X259E-X261E, X139R-X259E-X276W, X139R-X261D-X276W, X139R-X261E-X276W, X161G-X168T-X172F, X161G-X168T-X225Q, X161G-X168T-X259D, X161G-X168T-X259E, X161G-X168T-X261D, X161G-X168T-X261E, X161G-X168T-X276W, X161G-X172F-X225Q, X161G-X172F-X259D, X161G-X172F-X259E, X161G-X172F-X261D, X161G-X172F-X261E, X161G-X172F-X276W, X161G-X225Q-X259D, X161G-X225Q-X259E, X161G-X225Q-X261D, X161G-X225Q-X261E, X161G-X225Q-X276W, X161G-X259D-X261D, X161G-X259D-X261E, X161G-X259D-X276W, X161G-X259E-X261D, X161G-X259E-X261E, X161G-X259E-X276W, X161G-X261D-X276W, X161G-X261E-X276W, X168T-X172F-X225Q, X168T-X172F-X259D, X168T-X172F-X259E, X168T-X172F-X261D, X168T-X172F-X261E, X168T-X172F-X276W, X168T-X225Q-X259D, X168T-X225Q-X259E, X168T-X225Q-X261D, X168T-X225Q-X261E, X168T-X225Q-X276W, X168T-X259D-X261D, X168T-X259D-X261E, X168T-X259D-X276W, X168T-X259E-X261D, X168T-X259E-X261E, X168T-X259E-X276W, X168T-X261D-X276W, X168T-X261E-X276W, X172F-X225Q-X259D, X172F-X225Q-X259E, X172F-X225Q-X261D, X172F-X225Q-X261E, X172F-X225Q-X276W, X172F-X259D-X261D, X172F-X259D-X261E, X172F-X259D-X276W, X172F-X259E-X261D, X172F-X259E-X261E, X172F-X259E-X276W, X172F-X261D-X276W, X172F-X261E-X276W, X225Q-X259D-X261D, X225Q-X259D-X261E, X225Q-X259D-X276W, X225Q-X259E-X261D, X225Q-X259E-X261E, X225Q-X259E-X276W, X225Q-X261D-X276W, X225Q-X261E-X276W, X259D-X261D-X276W, X259D-X261E-X276W, X259E-X261D-X276W, and X259E-X261E-X276W.
25. A mannanase variant comprising amino acid substitutions selected from the group consisting of X0136P-X019D-X0225N-X032Y, X0136P-X019D-X0225N-X072V, X0136P-X019D-X0225N-X093Q, X0136P-X019D-X0225N-X131S, X0136P-X019D-X0225N-X139R, X0136P-X019D-X0225N-X161G, X0136P-X019D-X0225N-X168T, X0136P-X019D-X0225N-X172F, X0136P-X019D-X0225N-X259D, X0136P-X019D-X0225N-X259E, X0136P-X019D-X0225N-X261D, X0136P-X019D-X0225N-X261E, X0136P-X019D-X0225N-X276W, X0136P-X019D-X032Y-X072V, X0136P-X019D-X032Y-X093Q, X0136P-X019D-X032Y-X131S, X0136P-X019D-X032Y-X139R, X0136P-X019D-X032Y-X161G, X0136P-X019D-X032Y-X168T, X0136P-X019D-X032Y-X172F, X0136P-X019D-X032Y-X225Q, X0136P-X019D-X032Y-X259D, X0136P-X019D-X032Y-X259E, X0136P-X019D-X032Y-X261D, X0136P-X019D-X032Y-X261E, X0136P-X019D-X032Y-X276W, X0136P-X019D-X072V-X093Q, X0136P-X019D-X072V-X131S, X0136P-X019D-X072V-X139R, X0136P-X019D-X072V-X161G, X0136P-X019D-X072V-X168T, X0136P-X019D-X072V-X172F, X0136P-X019D-X072V-X225Q, X0136P-X019D-X072V-X259D, X0136P-X019D-X072V-X259E, X0136P-X019D-X072V-X261D, X0136P-X019D-X072V-X261E, X0136P-X019D-X072V-X276W, X0136P-X019D-X093Q-X131S, X0136P-X019D-X093Q-X139R, X0136P-X019D-X093Q-X161G, X0136P-X019D-X093Q-X168T, X0136P-X019D-X093Q-X172F, X0136P-X019D-X093Q-X225Q, X0136P-X019D-X093Q-X259D, X0136P-X019D-X093Q-X259E, X0136P-X019D-X093Q-X261D, X0136P-X019D-X093Q-X261E, X0136P-X019D-X093Q-X276W, X0136P-X019D-X131S-X139R, X0136P-X019D-X131S-X161G, X0136P-X019D-X131S-X168T, X0136P-X019D-X131S-X172F, X0136P-X019D-X131S-X225Q, X0136P-X019D-X131S-X259D, X0136P-X019D-X131S-X259E, X0136P-X019D-X131S-X261D, X0136P-X019D-X131S-X261E, X0136P-X019D-X131S-X276W, X0136P-X019D-X139R-X161G, X0136P-X019D-X139R-X168T, X0136P-X019D-X139R-X172F, X0136P-X019D-X139R-X225Q, X0136P-X019D-X139R-X259D, X0136P-X019D-X139R-X259E, X0136P-X019D-X139R-X261D, X0136P-X019D-X139R-X261E, X0136P-X019D-X139R-X276W, X0136P-X019D-X161G-X168T, X0136P-X019D-X161G-X172F, X0136P-X019D-X161G-X225Q, X0136P-X019D-X161G-X259D, X0136P-X019D-X161G-X259E, X0136P-X019D-X161G-X261D, X0136P-X019D-X161G-X261E, X0136P-X019D-X161G-X276W, X0136P-X019D-X168T-X172F, X0136P-X019D-X168T-X225Q, X0136P-X019D-X168T-X259D, X0136P-X019D-X168T-X259E, X0136P-X019D-X168T-X261D, X0136P-X019D-X168T-X261E, X0136P-X019D-X168T-X276W, X0136P-X019D-X172F-X225Q, X0136P-X019D-X172F-X259D, X0136P-X019D-X172F-X259E, X0136P-X019D-X172F-X261D, X0136P-X019D-X172F-X261E, X0136P-X019D-X172F-X276W, X0136P-X019D-X225Q-X259D, X0136P-X019D-X225Q-X259E, X0136P-X019D-X225Q-X261D, X0136P-X019D-X225Q-X261E, X0136P-X019D-X225Q-X276W, X0136P-X019D-X259D-X261D, X0136P-X019D-X259D-X261E, X0136P-X019D-X259D-X276W, X0136P-X019D-X259E-X261D, X0136P-X019D-X259E-X261E, X0136P-X019D-X259E-X276W, X0136P-X019D-X261D-X276W, X0136P-X019D-X261E-X276W, X0136P-X0225N-X032Y-X072V, X0136P-X0225N-X032Y-X093Q, X0136P-X0225N-X032Y-X131S, X0136P-X0225N-X032Y-X139R, X0136P-X0225N-X032Y-X161G, X0136P-X0225N-X032Y-X168T, X0136P-X0225N-X032Y-X172F, X0136P-X0225N-X032Y-X259D, X0136P-X0225N-X032Y-X259E, X0136P-X0225N-X032Y-X261D, X0136P-X0225N-X032Y-X261E, X0136P-X0225N-X032Y-X276W, X0136P-X0225N-X072V-X093Q, X0136P-X0225N-X072V-X131S, X0136P-X0225N-X072V-X139R, X0136P-X0225N-X072V-X161G, X0136P-X0225N-X072V-X168T, X0136P-X0225N-X072V-X172F, X0136P-X0225N-X072V-X259D, X0136P-X0225N-X072V-X259E, X0136P-X0225N-X072V-X261D, X0136P-X0225N-X072V-X261E, X0136P-X0225N-X072V-X276W, X0136P-X0225N-X093Q-X131S, X0136P-X0225N-X093Q-X139R, X0136P-X0225N-X093Q-X161G, X0136P-X0225N-X093Q-X168T, X0136P-X0225N-X093Q-X172F, X0136P-X0225N-X093Q-X259D, X0136P-X0225N-X093Q-X259E, X0136P-X0225N-X093Q-X261D, X0136P-X0225N-X093Q-X261E, X0136P-X0225N-X093Q-X276W, X0136P-X0225N-X131S-X139R, X0136P-X0225N-X131S-X161G, X0136P-X0225N-X131S-X168T, X0136P-X0225N-X131S-X172F, X0136P-X0225N-X131S-X259D, X0136P-X0225N-X131S-X259E, X0136P-X0225N-X131S-X261D, X0136P-X0225N-X131S-X261E, X0136P-X0225N-X131S-X276W, X0136P-X0225N-X139R-X161G, X0136P-X0225N-X139R-X168T, X0136P-X0225N-X139R-X172F, X0136P-X0225N-X139R-X259D, X0136P-X0225N-X139R-X259E, X0136P-X0225N-X139R-X261D, X0136P-X0225N-X139R-X261E, X0136P-X0225N-X139R-X276W, X0136P-X0225N-X161G-X168T, X0136P-X0225N-X161G-X172F, X0136P-X0225N-X161G-X259D, X0136P-X0225N-X161G-X259E, X0136P-X0225N-X161G-X261D, X0136P-X0225N-X161G-X261E, X0136P-X0225N-X161G-X276W, X0136P-X0225N-X168T-X172F, X0136P-X0225N-X168T-X259D, X0136P-X0225N-X168T-X259E, X0136P-X0225N-X168T-X261D, X0136P-X0225N-X168T-X261E, X0136P-X0225N-X168T-X276W, X0136P-X0225N-X172F-X259D, X0136P-X0225N-X172F-X259E, X0136P-X0225N-X172F-X261D, X0136P-X0225N-X172F-X261E, X0136P-X0225N-X172F-X276W, X0136P-X0225N-X259D-X261D, X0136P-X0225N-X259D-X261E, X0136P-X0225N-X259D-X276W, X0136P-X0225N-X259E-X261D, X0136P-X0225N-X259E-X261E, X0136P-X0225N-X259E-X276W, X0136P-X0225N-X261D-X276W, X0136P-X0225N-X261E-X276W, X0136P-X032Y-X072V-X093Q, X0136P-X032Y-X072V-X131S, X0136P-X032Y-X072V-X139R, X0136P-X032Y-X072V-X161G, X0136P-X032Y-X072V-X168T, X0136P-X032Y-X072V-X172F, X0136P-X032Y-X072V-X225Q, X0136P-X032Y-X072V-X259D, X0136P-X032Y-X072V-X259E, X0136P-X032Y-X072V-X261D, X0136P-X032Y-X072V-X261E, X0136P-X032Y-X072V-X276W, X0136P-X032Y-X093Q-X131S, X0136P-X032Y-X093Q-X139R, X0136P-X032Y-X093Q-X161G, X0136P-X032Y-X093Q-X168T, X0136P-X032Y-X093Q-X172F, X0136P-X032Y-X093Q-X225Q, X0136P-X032Y-X093Q-X259D, X0136P-X032Y-X093Q-X259E, X0136P-X032Y-X093Q-X261D, X0136P-X032Y-X093Q-X261E, X0136P-X032Y-X093Q-X276W, X0136P-X032Y-X131S-X139R, X0136P-X032Y-X131S-X161G, X0136P-X032Y-X131S-X168T, X0136P-X032Y-X131S-X172F, X0136P-X032Y-X131S-X225Q, X0136P-X032Y-X131S-X259D, X0136P-X032Y-X131S-X259E, X0136P-X032Y-X131S-X261D, X0136P-X032Y-X131S-X261E, X0136P-X032Y-X131S-X276W, X0136P-X032Y-X139R-X161G, X0136P-X032Y-X139R-X168T, X0136P-X032Y-X139R-X172F, X0136P-X032Y-X139R-X225Q, X0136P-X032Y-X139R-X259D, X0136P-X032Y-X139R-X259E, X0136P-X032Y-X139R-X261D, X0136P-X032Y-X139R-X261E, X0136P-X032Y-X139R-X276W, X0136P-X032Y-X161G-X168T, X0136P-X032Y-X161G-X172F, X0136P-X032Y-X161G-X225Q, X0136P-X032Y-X161G-X259D, X0136P-X032Y-X161G-X259E, X0136P-X032Y-X161G-X261D, X0136P-X032Y-X161G-X261E, X0136P-X032Y-X161G-X276W, X0136P-X032Y-X168T-X172F, X0136P-X032Y-X168T-X225Q, X0136P-X032Y-X168T-X259D, X0136P-X032Y-X168T-X259E, X0136P-X032Y-X168T-X261D, X0136P-X032Y-X168T-X261E, X0136P-X032Y-X168T-X276W, X0136P-X032Y-X172F-X225Q, X0136P-X032Y-X172F-X259D, X0136P-X032Y-X172F-X259E, X0136P-X032Y-X172F-X261D, X0136P-X032Y-X172F-X261E, X0136P-X032Y-X172F-X276W, X0136P-X032Y-X225Q-X259D, X0136P-X032Y-X225Q-X259E, X0136P-X032Y-X225Q-X261D, X0136P-X032Y-X225Q-X261E, X0136P-X032Y-X225Q-X276W, X0136P-X032Y-X259D-X261D, X0136P-X032Y-X259D-X261E, X0136P-X032Y-X259D-X276W, X0136P-X032Y-X259E-X261D, X0136P-X032Y-X259E-X261E, X0136P-X032Y-X259E-X276W, X0136P-X032Y-X261D-X276W, X0136P-X032Y-X261E-X276W, X0136P-X072V-X093Q-X131S, X0136P-X072V-X093Q-X139R, X0136P-X072V-X093Q-X161G, X0136P-X072V-X093Q-X168T, X0136P-X072V-X093Q-X172F, X0136P-X072V-X093Q-X225Q, X0136P-X072V-X093Q-X259D, X0136P-X072V-X093Q-X259E, X0136P-X072V-X093Q-X261D, X0136P-X072V-X093Q-X261E, X0136P-X072V-X093Q-X276W, X0136P-X072V-X131S-X139R, X0136P-X072V-X131S-X161G, X0136P-X072V-X131S-X168T, X0136P-X072V-X131S-X172F, X0136P-X072V-X131S-X225Q, X0136P-X072V-X131S-X259D, X0136P-X072V-X131S-X259E, X0136P-X072V-X131S-X261D, X0136P-X072V-X131S-X261E, X0136P-X072V-X131S-X276W, X0136P-X072V-X139R-X161G, X0136P-X072V-X139R-X168T, X0136P-X072V-X139R-X172F, X0136P-X072V-X139R-X225Q, X0136P-X072V-X139R-X259D, X0136P-X072V-X139R-X259E, X0136P-X072V-X139R-X261D, X0136P-X072V-X139R-X261E, X0136P-X072V-X139R-X276W, X0136P-X072V-X161G-X168T, X0136P-X072V-X161G-X172F, X0136P-X072V-X161G-X225Q, X0136P-X072V-X161G-X259D, X0136P-X072V-X161G-X259E, X0136P-X072V-X161G-X261D, X0136P-X072V-X161G-X261E, X0136P-X072V-X161G-X276W, X0136P-X072V-X168T-X172F, X0136P-X072V-X168T-X225Q, X0136P-X072V-X168T-X259D, X0136P-X072V-X168T-X259E, X0136P-X072V-X168T-X261D, X0136P-X072V-X168T-X261E, X0136P-X072V-X168T-X276W, X0136P-X072V-X172F-X225Q, X0136P-X072V-X172F-X259D, X0136P-X072V-X172F-X259E, X0136P-X072V-X172F-X261D, X0136P-X072V-X172F-X261E, X0136P-X072V-X172F-X276W, X0136P-X072V-X225Q-X259D, X0136P-X072V-X225Q-X259E, X0136P-X072V-X225Q-X261D, X0136P-X072V-X225Q-X261E, X0136P-X072V-X225Q-X276W, X0136P-X072V-X259D-X261D, X0136P-X072V-X259D-X261E, X0136P-X072V-X259D-X276W, X0136P-X072V-X259E-X261D, X0136P-X072V-X259E-X261E, X0136P-X072V-X259E-X276W, X0136P-X072V-X261D-X276W, X0136P-X072V-X261E-X276W, X0136P-X093Q-X131S-X139R, X0136P-X093Q-X131S-X161G, X0136P-X093Q-X131S-X168T, X0136P-X093Q-X131S-X172F, X0136P-X093Q-X131S-X225Q, X0136P-X093Q-X131S-X259D, X0136P-X093Q-X131S-X259E, X0136P-X093Q-X131S-X261D, X0136P-X093Q-X131S-X261E, X0136P-X093Q-X131S-X276W, X0136P-X093Q-X139R-X161G, X0136P-X093Q-X139R-X168T, X0136P-X093Q-X139R-X172F, X0136P-X093Q-X139R-X225Q, X0136P-X093Q-X139R-X259D, X0136P-X093Q-X139R-X259E, X0136P-X093Q-X139R-X261D, X0136P-X093Q-X139R-X261E, X0136P-X093Q-X139R-X276W, X0136P-X093Q-X161G-X168T, X0136P-X093Q-X161G-X172F, X0136P-X093Q-X161G-X225Q, X0136P-X093Q-X161G-X259D, X0136P-X093Q-X161G-X259E, X0136P-X093Q-X161G-X261D, X0136P-X093Q-X161G-X261E, X0136P-X093Q-X161G-X276W, X0136P-X093Q-X168T-X172F, X0136P-X093Q-X168T-X225Q, X0136P-X093Q-X168T-X259D, X0136P-X093Q-X168T-X259E, X0136P-X093Q-X168T-X261D, X0136P-X093Q-X168T-X261E, X0136P-X093Q-X168T-X276W, X0136P-X093Q-X172F-X225Q, X0136P-X093Q-X172F-X259D, X0136P-X093Q-X172F-X259E, X0136P-X093Q-X172F-X261D, X0136P-X093Q-X172F-X261E, X0136P-X093Q-X172F-X276W, X0136P-X093Q-X225Q-X259D, X0136P-X093Q-X225Q-X259E, X0136P-X093Q-X225Q-X261D, X0136P-X093Q-X225Q-X261E, X0136P-X093Q-X225Q-X276W, X0136P-X093Q-X259D-X261D, X0136P-X093Q-X259D-X261E, X0136P-X093Q-X259D-X276W, X0136P-X093Q-X259E-X261D, X0136P-X093Q-X259E-X261E, X0136P-X093Q-X259E-X276W, X0136P-X093Q-X261D-X276W, X0136P-X093Q-X261E-X276W, X0136P-X131S-X139R-X161G, X0136P-X131S-X139R-X168T, X0136P-X131S-X139R-X172F, X0136P-X131S-X139R-X225Q, X0136P-X131S-X139R-X259D, X0136P-X131S-X139R-X259E, X0136P-X131S-X139R-X261D, X0136P-X131S-X139R-X261E, X0136P-X131S-X139R-X276W, X0136P-X131S-X161G-X168T, X0136P-X131S-X161G-X172F, X0136P-X131S-X161G-X225Q, X0136P-X131S-X161G-X259D, X0136P-X131S-X161G-X259E, X0136P-X131S-X161G-X261D, X0136P-X131S-X161G-X261E, X0136P-X131S-X161G-X276W, X0136P-X131S-X168T-X172F, X0136P-X131S-X168T-X225Q, X0136P-X131S-X168T-X259D, X0136P-X131S-X168T-X259E, X0136P-X131S-X168T-X261D, X0136P-X131S-X168T-X261E, X0136P-X131S-X168T-X276W, X0136P-X131S-X172F-X225Q, X0136P-X131S-X172F-X259D, X0136P-X131S-X172F-X259E, X0136P-X131S-X172F-X261D, X0136P-X131S-X172F-X261E, X0136P-X131S-X172F-X276W, X0136P-X131S-X225Q-X259D, X0136P-X131S-X225Q-X259E, X0136P-X131S-X225Q-X261D, X0136P-X131S-X225Q-X261E, X0136P-X131S-X225Q-X276W, X0136P-X131S-X259D-X261D, X0136P-X131S-X259D-X261E, X0136P-X131S-X259D-X276W, X0136P-X131S-X259E-X261D, X0136P-X131S-X259E-X261E, X0136P-X131S-X259E-X276W, X0136P-X131S-X261D-X276W, X0136P-X131S-X261E-X276W, X0136P-X139R-X161G-X168T, X0136P-X139R-X161G-X172F, X0136P-X139R-X161G-X225Q, X0136P-X139R-X161G-X259D, X0136P-X139R-X161G-X259E, X0136P-X139R-X161G-X261D, X0136P-X139R-X161G-X261E, X0136P-X139R-X161G-X276W, X0136P-X139R-X168T-X172F, X0136P-X139R-X168T-X225Q, X0136P-X139R-X168T-X259D, X0136P-X139R-X168T-X259E, X0136P-X139R-X168T-X261D, X0136P-X139R-X168T-X261E, X0136P-X139R-X168T-X276W, X0136P-X139R-X172F-X225Q, X0136P-X139R-X172F-X259D, X0136P-X139R-X172F-X259E, X0136P-X139R-X172F-X261D, X0136P-X139R-X172F-X261E, X0136P-X139R-X172F-X276W, X0136P-X139R-X225Q-X259D, X0136P-X139R-X225Q-X259E, X0136P-X139R-X225Q-X261D, X0136P-X139R-X225Q-X261E, X0136P-X139R-X225Q-X276W, X0136P-X139R-X259D-X261D, X0136P-X139R-X259D-X261E, X0136P-X139R-X259D-X276W, X0136P-X139R-X259E-X261D, X0136P-X139R-X259E-X261E, X0136P-X139R-X259E-X276W, X0136P-X139R-X261D-X276W, X0136P-X139R-X261E-X276W, X0136P-X161G-X168T-X172F, X0136P-X161G-X168T-X225Q, X0136P-X161G-X168T-X259D, X0136P-X161G-X168T-X259E, X0136P-X161G-X168T-X261D, X0136P-X161G-X168T-X261E, X0136P-X161G-X168T-X276W, X0136P-X161G-X172F-X225Q, X0136P-X161G-X172F-X259D, X0136P-X161G-X172F-X259E, X0136P-X161G-X172F-X261D, X0136P-X161G-X172F-X261E, X0136P-X161G-X172F-X276W, X0136P-X161G-X225Q-X259D, X0136P-X161G-X225Q-X259E, X0136P-X161G-X225Q-X261D, X0136P-X161G-X225Q-X261E, X0136P-X161G-X225Q-X276W, X0136P-X161G-X259D-X261D, X0136P-X161G-X259D-X261E, X0136P-X161G-X259D-X276W, X0136P-X161G-X259E-X261D, X0136P-X161G-X259E-X261E, X0136P-X161G-X259E-X276W, X0136P-X161G-X261D-X276W, X0136P-X161G-X261E-X276W, X0136P-X168T-X172F-X225Q, X0136P-X168T-X172F-X259D, X0136P-X168T-X172F-X259E, X0136P-X168T-X172F-X261D, X0136P-X168T-X172F-X261E, X0136P-X168T-X172F-X276W, X0136P-X168T-X225Q-X259D, X0136P-X168T-X225Q-X259E, X0136P-X168T-X225Q-X261D, X0136P-X168T-X225Q-X261E, X0136P-X168T-X225Q-X276W, X0136P-X168T-X259D-X261D, X0136P-X168T-X259D-X261E, X0136P-X168T-X259D-X276W, X0136P-X168T-X259E-X261D, X0136P-X168T-X259E-X261E, X0136P-X168T-X259E-X276W, X0136P-X168T-X261D-X276W, X0136P-X168T-X261E-X276W, X0136P-X172F-X225Q-X259D, X0136P-X172F-X225Q-X259E, X0136P-X172F-X225Q-X261D, X0136P-X172F-X225Q-X261E, X0136P-X172F-X225Q-X276W, X0136P-X172F-X259D-X261D, X0136P-X172F-X259D-X261E, X0136P-X172F-X259D-X276W, X0136P-X172F-X259E-X261D, X0136P-X172F-X259E-X261E, X0136P-X172F-X259E-X276W, X0136P-X172F-X261D-X276W, X0136P-X172F-X261E-X276W, X0136P-X225Q-X259D-X261D, X0136P-X225Q-X259D-X261E, X0136P-X225Q-X259D-X276W, X0136P-X225Q-X259E-X261D, X0136P-X225Q-X259E-X261E, X0136P-X225Q-X259E-X276W, X0136P-X225Q-X261D-X276W, X0136P-X225Q-X261E-X276W, X0136P-X259D-X261D-X276W, X0136P-X259D-X261E-X276W, X0136P-X259E-X261D-X276W, X0136P-X259E-X261E-X276W, X019D-X0225N-X032Y-X072V, X019D-X0225N-X032Y-X093Q, X019D-X0225N-X032Y-X131S, X019D-X0225N-X032Y-X139R, X019D-X0225N-X032Y-X161G, X019D-X0225N-X032Y-X168T, X019D-X0225N-X032Y-X172F, X019D-X0225N-X032Y-X259D, X019D-X0225N-X032Y-X259E, X019D-X0225N-X032Y-X261D, X019D-X0225N-X032Y-X261E, X019D-X0225N-X032Y-X276W, X019D-X0225N-X072V-X093Q, X019D-X0225N-X072V-X131S, X019D-X0225N-X072V-X139R, X019D-X0225N-X072V-X161G, X019D-X0225N-X072V-X168T, X019D-X0225N-X072V-X172F, X019D-X0225N-X072V-X259D, X019D-X0225N-X072V-X259E, X019D-X0225N-X072V-X261D, X019D-X0225N-X072V-X261E, X019D-X0225N-X072V-X276W, X019D-X0225N-X093Q-X131S, X019D-X0225N-X093Q-X139R, X019D-X0225N-X093Q-X161G, X019D-X0225N-X093Q-X168T, X019D-X0225N-X093Q-X172F, X019D-X0225N-X093Q-X259D, X019D-X0225N-X093Q-X259E, X019D-X0225N-X093Q-X261D, X019D-X0225N-X093Q-X261E, X019D-X0225N-X093Q-X276W, X019D-X0225N-X131S-X139R, X019D-X0225N-X131S-X161G, X019D-X0225N-X131S-X168T, X019D-X0225N-X131S-X172F, X019D-X0225N-X131S-X259D, X019D-X0225N-X131S-X259E, X019D-X0225N-X131S-X261D, X019D-X0225N-X131S-X261E, X019D-X0225N-X131S-X276W, X019D-X0225N-X139R-X161G, X019D-X0225N-X139R-X168T, X019D-X0225N-X139R-X172F, X019D-X0225N-X139R-X259D, X019D-X0225N-X139R-X259E, X019D-X0225N-X139R-X261D, X019D-X0225N-X139R-X261E, X019D-X0225N-X139R-X276W, X019D-X0225N-X161G-X168T, X019D-X0225N-X161G-X172F, X019D-X0225N-X161G-X259D, X019D-X0225N-X161G-X259E, X019D-X0225N-X161G-X261D, X019D-X0225N-X161G-X261E, X019D-X0225N-X161G-X276W, X019D-X0225N-X168T-X172F, X019D-X0225N-X168T-X259D, X019D-X0225N-X168T-X259E, X019D-X0225N-X168T-X261D, X019D-X0225N-X168T-X261E, X019D-X0225N-X168T-X276W, X019D-X0225N-X172F-X259D, X019D-X0225N-X172F-X259E, X019D-X0225N-X172F-X261D, X019D-X0225N-X172F-X261E, X019D-X0225N-X172F-X276W, X019D-X0225N-X259D-X261D, X019D-X0225N-X259D-X261E, X019D-X0225N-X259D-X276W, X019D-X0225N-X259E-X261D, X019D-X0225N-X259E-X261E, X019D-X0225N-X259E-X276W, X019D-X0225N-X261D-X276W, X019D-X0225N-X261E-X276W, X019D-X032Y-X072V-X093Q, X019D-X032Y-X072V-X131S, X019D-X032Y-X072V-X139R, X019D-X032Y-X072V-X161G, X019D-X032Y-X072V-X168T, X019D-X032Y-X072V-X172F, X019D-X032Y-X072V-X225Q, X019D-X032Y-X072V-X259D, X019D-X032Y-X072V-X259E, X019D-X032Y-X072V-X261D, X019D-X032Y-X072V-X261E, X019D-X032Y-X072V-X276W, X019D-X032Y-X093Q-X131S, X019D-X032Y-X093Q-X139R, X019D-X032Y-X093Q-X161G, X019D-X032Y-X093Q-X168T, X019D-X032Y-X093Q-X172F, X019D-X032Y-X093Q-X225Q, X019D-X032Y-X093Q-X259D, X019D-X032Y-X093Q-X259E, X019D-X032Y-X093Q-X261D, X019D-X032Y-X093Q-X261E, X019D-X032Y-X093Q-X276W, X019D-X032Y-X131S-X139R, X019D-X032Y-X131S-X161G, X019D-X032Y-X131S-X168T, X019D-X032Y-X131S-X172F, X019D-X032Y-X131S-X225Q, X019D-X032Y-X131S-X259D, X019D-X032Y-X131S-X259E, X019D-X032Y-X131S-X261D, X019D-X032Y-X131S-X261E, X019D-X032Y-X131S-X276W, X019D-X032Y-X139R-X161G, X019D-X032Y-X139R-X168T, X019D-X032Y-X139R-X172F, X019D-X032Y-X139R-X225Q, X019D-X032Y-X139R-X259D, X019D-X032Y-X139R-X259E, X019D-X032Y-X139R-X261D, X019D-X032Y-X139R-X261E, X019D-X032Y-X139R-X276W, X019D-X032Y-X161G-X168T, X019D-X032Y-X161G-X172F, X019D-X032Y-X161G-X225Q, X019D-X032Y-X161G-X259D, X019D-X032Y-X161G-X259E, X019D-X032Y-X161G-X261D, X019D-X032Y-X161G-X261E, X019D-X032Y-X161G-X276W, X019D-X032Y-X168T-X172F, X019D-X032Y-X168T-X225Q, X019D-X032Y-X168T-X259D, X019D-X032Y-X168T-X259E, X019D-X032Y-X168T-X261D, X019D-X032Y-X168T-X261E, X019D-X032Y-X168T-X276W, X019D-X032Y-X172F-X225Q, X019D-X032Y-X172F-X259D, X019D-X032Y-X172F-X259E, X019D-X032Y-X172F-X261D, X019D-X032Y-X172F-X261E, X019D-X032Y-X172F-X276W, X019D-X032Y-X225Q-X259D, X019D-X032Y-X225Q-X259E, X019D-X032Y-X225Q-X261D, X019D-X032Y-X225Q-X261E, X019D-X032Y-X225Q-X276W, X019D-X032Y-X259D-X261D, X019D-X032Y-X259D-X261E, X019D-X032Y-X259D-X276W, X019D-X032Y-X259E-X261D, X019D-X032Y-X259E-X261E, X019D-X032Y-X259E-X276W, X019D-X032Y-X261D-X276W, X019D-X032Y-X261E-X276W, X019D-X072V-X093Q-X131S, X019D-X072V-X093Q-X139R, X019D-X072V-X093Q-X161G, X019D-X072V-X093Q-X168T, X019D-X072V-X093Q-X172F, X019D-X072V-X093Q-X225Q, X019D-X072V-X093Q-X259D, X019D-X072V-X093Q-X259E, X019D-X072V-X093Q-X261D, X019D-X072V-X093Q-X261E, X019D-X072V-X093Q-X276W, X019D-X072V-X131S-X139R, X019D-X072V-X131S-X161G, X019D-X072V-X131S-X168T, X019D-X072V-X131S-X172F, X019D-X072V-X131S-X225Q, X019D-X072V-X131S-X259D, X019D-X072V-X131S-X259E, X019D-X072V-X131S-X261D, X019D-X072V-X131S-X261E, X019D-X072V-X131S-X276W, X019D-X072V-X139R-X161G, X019D-X072V-X139R-X168T, X019D-X072V-X139R-X172F, X019D-X072V-X139R-X225Q, X019D-X072V-X139R-X259D, X019D-X072V-X139R-X259E, X019D-X072V-X139R-X261D, X019D-X072V-X139R-X261E, X019D-X072V-X139R-X276W, X019D-X072V-X161G-X168T, X019D-X072V-X161G-X172F, X019D-X072V-X161G-X225Q, X019D-X072V-X161G-X259D, X019D-X072V-X161G-X259E, X019D-X072V-X161G-X261D, X019D-X072V-X161G-X261E, X019D-X072V-X161G-X276W, X019D-X072V-X168T-X172F, X019D-X072V-X168T-X225Q, X019D-X072V-X168T-X259D, X019D-X072V-X168T-X259E, X019D-X072V-X168T-X261D, X019D-X072V-X168T-X261E, X019D-X072V-X168T-X276W, X019D-X072V-X172F-X225Q, X019D-X072V-X172F-X259D, X019D-X072V-X172F-X259E, X019D-X072V-X172F-X261D, X019D-X072V-X172F-X261E, X019D-X072V-X172F-X276W, X019D-X072V-X225Q-X259D, X019D-X072V-X225Q-X259E, X019D-X072V-X225Q-X261D, X019D-X072V-X225Q-X261E, X019D-X072V-X225Q-X276W, X019D-X072V-X259D-X261D, X019D-X072V-X259D-X261E, X019D-X072V-X259D-X276W, X019D-X072V-X259E-X261D, X019D-X072V-X259E-X261E, X019D-X072V-X259E-X276W, X019D-X072V-X261D-X276W, X019D-X072V-X261E-X276W, X019D-X093Q-X131S-X139R, X019D-X093Q-X131S-X161G, X019D-X093Q-X131S-X168T, X019D-X093Q-X131S-X172F, X019D-X093Q-X131S-X225Q, X019D-X093Q-X131S-X259D, X019D-X093Q-X131S-X259E, X019D-X093Q-X131S-X261D, X019D-X093Q-X131S-X261E, X019D-X093Q-X131S-X276W, X019D-X093Q-X139R-X161G, X019D-X093Q-X139R-X168T, X019D-X093Q-X139R-X172F, X019D-X093Q-X139R-X225Q, X019D-X093Q-X139R-X259D, X019D-X093Q-X139R-X259E, X019D-X093Q-X139R-X261D, X019D-X093Q-X139R-X261E, X019D-X093Q-X139R-X276W, X019D-X093Q-X161G-X168T, X019D-X093Q-X161G-X172F, X019D-X093Q-X161G-X225Q, X019D-X093Q-X161G-X259D, X019D-X093Q-X161G-X259E, X019D-X093Q-X161G-X261D, X019D-X093Q-X161G-X261E, X019D-X093Q-X161G-X276W, X019D-X093Q-X168T-X172F, X019D-X093Q-X168T-X225Q, X019D-X093Q-X168T-X259D, X019D-X093Q-X168T-X259E, X019D-X093Q-X168T-X261D, X019D-X093Q-X168T-X261E, X019D-X093Q-X168T-X276W, X019D-X093Q-X172F-X225Q, X019D-X093Q-X172F-X259D, X019D-X093Q-X172F-X259E, X019D-X093Q-X172F-X261D, X019D-X093Q-X172F-X261E, X019D-X093Q-X172F-X276W, X019D-X093Q-X225Q-X259D, X019D-X093Q-X225Q-X259E, X019D-X093Q-X225Q-X261D, X019D-X093Q-X225Q-X261E, X019D-X093Q-X225Q-X276W, X019D-X093Q-X259D-X261D, X019D-X093Q-X259D-X261E, X019D-X093Q-X259D-X276W, X019D-X093Q-X259E-X261D, X019D-X093Q-X259E-X261E, X019D-X093Q-X259E-X276W, X019D-X093Q-X261D-X276W, X019D-X093Q-X261E-X276W, X019D-X131S-X139R-X161G, X019D-X131S-X139R-X168T, X019D-X131S-X139R-X172F, X019D-X131S-X139R-X225Q, X019D-X131S-X139R-X259D, X019D-X131S-X139R-X259E, X019D-X131S-X139R-X261D, X019D-X131S-X139R-X261E, X019D-X131S-X139R-X276W, X019D-X131S-X161G-X168T, X019D-X131S-X161G-X172F, X019D-X131S-X161G-X225Q, X019D-X131S-X161G-X259D, X019D-X131S-X161G-X259E, X019D-X131S-X161G-X261D, X019D-X131S-X161G-X261E, X019D-X131S-X161G-X276W, X019D-X131S-X168T-X172F, X019D-X131S-X168T-X225Q, X019D-X131S-X168T-X259D, X019D-X131S-X168T-X259E, X019D-X131S-X168T-X261D, X019D-X131S-X168T-X261E, X019D-X131S-X168T-X276W, X019D-X131S-X172F-X225Q, X019D-X131S-X172F-X259D, X019D-X131S-X172F-X259E, X019D-X131S-X172F-X261D, X019D-X131S-X172F-X261E, X019D-X131S-X172F-X276W, X019D-X131S-X225Q-X259D, X019D-X131S-X225Q-X259E, X019D-X131S-X225Q-X261D, X019D-X131S-X225Q-X261E, X019D-X131S-X225Q-X276W, X019D-X131S-X259D-X261D, X019D-X131S-X259D-X261E, X019D-X131S-X259D-X276W, X019D-X131S-X259E-X261D, X019D-X131S-X259E-X261E, X019D-X131S-X259E-X276W, X019D-X131S-X261D-X276W, X019D-X131S-X261E-X276W, X019D-X139R-X161G-X168T, X019D-X139R-X161G-X172F, X019D-X139R-X161G-X225Q, X019D-X139R-X161G-X259D, X019D-X139R-X161G-X259E, X019D-X139R-X161G-X261D, X019D-X139R-X161G-X261E, X019D-X139R-X161G-X276W, X019D-X139R-X168T-X172F, X019D-X139R-X168T-X225Q, X019D-X139R-X168T-X259D, X019D-X139R-X168T-X259E, X019D-X139R-X168T-X261D, X019D-X139R-X168T-X261E, X019D-X139R-X168T-X276W, X019D-X139R-X172F-X225Q, X019D-X139R-X172F-X259D, X019D-X139R-X172F-X259E, X019D-X139R-X172F-X261D, X019D-X139R-X172F-X261E, X019D-X139R-X172F-X276W, X019D-X139R-X225Q-X259D, X019D-X139R-X225Q-X259E, X019D-X139R-X225Q-X261D, X019D-X139R-X225Q-X261E, X019D-X139R-X225Q-X276W, X019D-X139R-X259D-X261D, X019D-X139R-X259D-X261E, X019D-X139R-X259D-X276W, X019D-X139R-X259E-X261D, X019D-X139R-X259E-X261E, X019D-X139R-X259E-X276W, X019D-X139R-X261D-X276W, X019D-X139R-X261E-X276W, X019D-X161G-X168T-X172F, X019D-X161G-X168T-X225Q, X019D-X161G-X168T-X259D, X019D-X161G-X168T-X259E, X019D-X161G-X168T-X261D, X019D-X161G-X168T-X261E, X019D-X161G-X168T-X276W, X019D-X161G-X172F-X225Q, X019D-X161G-X172F-X259D, X019D-X161G-X172F-X259E, X019D-X161G-X172F-X261D, X019D-X161G-X172F-X261E, X019D-X161G-X172F-X276W, X019D-X161G-X225Q-X259D, X019D-X161G-X225Q-X259E, X019D-X161G-X225Q-X261D, X019D-X161G-X225Q-X261E, X019D-X161G-X225Q-X276W, X019D-X161G-X259D-X261D, X019D-X161G-X259D-X261E, X019D-X161G-X259D-X276W, X019D-X161G-X259E-X261D, X019D-X161G-X259E-X261E, X019D-X161G-X259E-X276W, X019D-X161G-X261D-X276W, X019D-X161G-X261E-X276W, X019D-X168T-X172F-X225Q, X019D-X168T-X172F-X259D, X019D-X168T-X172F-X259E, X019D-X168T-X172F-X261D, X019D-X168T-X172F-X261E, X019D-X168T-X172F-X276W, X019D-X168T-X225Q-X259D, X019D-X168T-X225Q-X259E, X019D-X168T-X225Q-X261D, X019D-X168T-X225Q-X261E, X019D-X168T-X225Q-X276W, X019D-X168T-X259D-X261D, X019D-X168T-X259D-X261E, X019D-X168T-X259D-X276W, X019D-X168T-X259E-X261D, X019D-X168T-X259E-X261E, X019D-X168T-X259E-X276W, X019D-X168T-X261D-X276W, X019D-X168T-X261E-X276W, X019D-X172F-X225Q-X259D, X019D-X172F-X225Q-X259E, X019D-X172F-X225Q-X261D, X019D-X172F-X225Q-X261E, X019D-X172F-X225Q-X276W, X019D-X172F-X259D-X261D, X019D-X172F-X259D-X261E, X019D-X172F-X259D-X276W, X019D-X172F-X259E-X261D, X019D-X172F-X259E-X261E, X019D-X172F-X259E-X276W, X019D-X172F-X261D-X276W, X019D-X172F-X261E-X276W, X019D-X225Q-X259D-X261D, X019D-X225Q-X259D-X261E, X019D-X225Q-X259D-X276W, X019D-X225Q-X259E-X261D, X019D-X225Q-X259E-X261E, X019D-X225Q-X259E-X276W, X019D-X225Q-X261D-X276W, X019D-X225Q-X261E-X276W, X019D-X259D-X261D-X276W, X019D-X259D-X261E-X276W, X019D-X259E-X261D-X276W, X019D-X259E-X261E-X276W, X0225N-X032Y-X072V-X093Q, X0225N-X032Y-X072V-X131S, X0225N-X032Y-X072V-X139R, X0225N-X032Y-X072V-X161G, X0225N-X032Y-X072V-X168T, X0225N-X032Y-X072V-X172F, X0225N-X032Y-X072V-X259D, X0225N-X032Y-X072V-X259E, X0225N-X032Y-X072V-X261D, X0225N-X032Y-X072V-X261E, X0225N-X032Y-X072V-X276W, X0225N-X032Y-X093Q-X131S, X0225N-X032Y-X093Q-X139R, X0225N-X032Y-X093Q-X161G, X0225N-X032Y-X093Q-X168T, X0225N-X032Y-X093Q-X172F, X0225N-X032Y-X093Q-X259D, X0225N-X032Y-X093Q-X259E, X0225N-X032Y-X093Q-X261D, X0225N-X032Y-X093Q-X261E, X0225N-X032Y-X093Q-X276W, X0225N-X032Y-X131S-X139R, X0225N-X032Y-X131S-X161G, X0225N-X032Y-X131S-X168T, X0225N-X032Y-X131S-X172F, X0225N-X032Y-X131S-X259D, X0225N-X032Y-X131S-X259E, X0225N-X032Y-X131S-X261D, X0225N-X032Y-X131S-X261E, X0225N-X032Y-X131S-X276W, X0225N-X032Y-X139R-X161G, X0225N-X032Y-X139R-X168T, X0225N-X032Y-X139R-X172F, X0225N-X032Y-X139R-X259D, X0225N-X032Y-X139R-X259E, X0225N-X032Y-X139R-X261D, X0225N-X032Y-X139R-X261E, X0225N-X032Y-X139R-X276W, X0225N-X032Y-X161G-X168T, X0225N-X032Y-X161G-X172F, X0225N-X032Y-X161G-X259D, X0225N-X032Y-X161G-X259E, X0225N-X032Y-X161G-X261D, X0225N-X032Y-X161G-X261E, X0225N-X032Y-X161G-X276W, X0225N-X032Y-X168T-X172F, X0225N-X032Y-X168T-X259D, X0225N-X032Y-X168T-X259E, X0225N-X032Y-X168T-X261D, X0225N-X032Y-X168T-X261E, X0225N-X032Y-X168T-X276W, X0225N-X032Y-X172F-X259D, X0225N-X032Y-X172F-X259E, X0225N-X032Y-X172F-X261D, X0225N-X032Y-X172F-X261E, X0225N-X032Y-X172F-X276W, X0225N-X032Y-X259D-X261D, X0225N-X032Y-X259D-X261E, X0225N-X032Y-X259D-X276W, X0225N-X032Y-X259E-X261D, X0225N-X032Y-X259E-X261E, X0225N-X032Y-X259E-X276W, X0225N-X032Y-X261D-X276W, X0225N-X032Y-X261E-X276W, X0225N-X072V-X093Q-X131S, X0225N-X072V-X093Q-X139R, X0225N-X072V-X093Q-X161G, X0225N-X072V-X093Q-X168T, X0225N-X072V-X093Q-X172F, X0225N-X072V-X093Q-X259D, X0225N-X072V-X093Q-X259E, X0225N-X072V-X093Q-X261D, X0225N-X072V-X093Q-X261E, X0225N-X072V-X093Q-X276W, X0225N-X072V-X131S-X139R, X0225N-X072V-X131S-X161G, X0225N-X072V-X131S-X168T, X0225N-X072V-X131S-X172F, X0225N-X072V-X131S-X259D, X0225N-X072V-X131S-X259E, X0225N-X072V-X131S-X261D, X0225N-X072V-X131S-X261E, X0225N-X072V-X131S-X276W, X0225N-X072V-X139R-X161G, X0225N-X072V-X139R-X168T, X0225N-X072V-X139R-X172F, X0225N-X072V-X139R-X259D, X0225N-X072V-X139R-X259E, X0225N-X072V-X139R-X261D, X0225N-X072V-X139R-X261E, X0225N-X072V-X139R-X276W, X0225N-X072V-X161G-X168T, X0225N-X072V-X161G-X172F, X0225N-X072V-X161G-X259D, X0225N-X072V-X161G-X259E, X0225N-X072V-X161G-X261D, X0225N-X072V-X161G-X261E, X0225N-X072V-X161G-X276W, X0225N-X072V-X168T-X172F, X0225N-X072V-X168T-X259D, X0225N-X072V-X168T-X259E, X0225N-X072V-X168T-X261D, X0225N-X072V-X168T-X261E, X0225N-X072V-X168T-X276W, X0225N-X072V-X172F-X259D, X0225N-X072V-X172F-X259E, X0225N-X072V-X172F-X261D, X0225N-X072V-X172F-X261E, X0225N-X072V-X172F-X276W, X0225N-X072V-X259D-X261D, X0225N-X072V-X259D-X261E, X0225N-X072V-X259D-X276W, X0225N-X072V-X259E-X261D, X0225N-X072V-X259E-X261E, X0225N-X072V-X259E-X276W, X0225N-X072V-X261D-X276W, X0225N-X072V-X261E-X276W, X0225N-X093Q-X131S-X139R, X0225N-X093Q-X131S-X161G, X0225N-X093Q-X131S-X168T, X0225N-X093Q-X131S-X172F, X0225N-X093Q-X131S-X259D, X0225N-X093Q-X131S-X259E, X0225N-X093Q-X131S-X261D, X0225N-X093Q-X131S-X261E, X0225N-X093Q-X131S-X276W, X0225N-X093Q-X139R-X161G, X0225N-X093Q-X139R-X168T, X0225N-X093Q-X139R-X172F, X0225N-X093Q-X139R-X259D, X0225N-X093Q-X139R-X259E, X0225N-X093Q-X139R-X261D, X0225N-X093Q-X139R-X261E, X0225N-X093Q-X139R-X276W, X0225N-X093Q-X161G-X168T, X0225N-X093Q-X161G-X172F, X0225N-X093Q-X161G-X259D, X0225N-X093Q-X161G-X259E, X0225N-X093Q-X161G-X261D, X0225N-X093Q-X161G-X261E, X0225N-X093Q-X161G-X276W, X0225N-X093Q-X168T-X172F, X0225N-X093Q-X168T-X259D, X0225N-X093Q-X168T-X259E, X0225N-X093Q-X168T-X261D, X0225N-X093Q-X168T-X261E, X0225N-X093Q-X168T-X276W, X0225N-X093Q-X172F-X259D, X0225N-X093Q-X172F-X259E, X0225N-X093Q-X172F-X261D, X0225N-X093Q-X172F-X261E, X0225N-X093Q-X172F-X276W, X0225N-X093Q-X259D-X261D, X0225N-X093Q-X259D-X261E, X0225N-X093Q-X259D-X276W, X0225N-X093Q-X259E-X261D, X0225N-X093Q-X259E-X261E, X0225N-X093Q-X259E-X276W, X0225N-X093Q-X261D-X276W, X0225N-X093Q-X261E-X276W, X0225N-X131S-X139R-X161G, X0225N-X131S-X139R-X168T, X0225N-X131S-X139R-X172F, X0225N-X131S-X139R-X259D, X0225N-X131S-X139R-X259E, X0225N-X131S-X139R-X261D, X0225N-X131S-X139R-X261E, X0225N-X131S-X139R-X276W, X0225N-X131S-X161G-X168T, X0225N-X131S-X161G-X172F, X0225N-X131S-X161G-X259D, X0225N-X131S-X161G-X259E, X0225N-X131S-X161G-X261D, X0225N-X131S-X161G-X261E, X0225N-X131S-X161G-X276W, X0225N-X131S-X168T-X172F, X0225N-X131S-X168T-X259D, X0225N-X131S-X168T-X259E, X0225N-X131S-X168T-X261D, X0225N-X131S-X168T-X261E, X0225N-X131S-X168T-X276W, X0225N-X131S-X172F-X259D, X0225N-X131S-X172F-X259E, X0225N-X131S-X172F-X261D, X0225N-X131S-X172F-X261E, X0225N-X131S-X172F-X276W, X0225N-X131S-X259D-X261D, X0225N-X131S-X259D-X261E, X0225N-X131S-X259D-X276W, X0225N-X131S-X259E-X261D, X0225N-X131S-X259E-X261E, X0225N-X131S-X259E-X276W, X0225N-X131S-X261D-X276W, X0225N-X131S-X261E-X276W, X0225N-X139R-X161G-X168T, X0225N-X139R-X161G-X172F, X0225N-X139R-X161G-X259D, X0225N-X139R-X161G-X259E, X0225N-X139R-X161G-X261D, X0225N-X139R-X161G-X261E, X0225N-X139R-X161G-X276W, X0225N-X139R-X168T-X172F, X0225N-X139R-X168T-X259D, X0225N-X139R-X168T-X259E, X0225N-X139R-X168T-X261D, X0225N-X139R-X168T-X261E, X0225N-X139R-X168T-X276W, X0225N-X139R-X172F-X259D, X0225N-X139R-X172F-X259E, X0225N-X139R-X172F-X261D, X0225N-X139R-X172F-X261E, X0225N-X139R-X172F-X276W, X0225N-X139R-X259D-X261D, X0225N-X139R-X259D-X261E, X0225N-X139R-X259D-X276W, X0225N-X139R-X259E-X261D, X0225N-X139R-X259E-X261E, X0225N-X139R-X259E-X276W, X0225N-X139R-X261D-X276W, X0225N-X139R-X261E-X276W, X0225N-X161G-X168T-X172F, X0225N-X161G-X168T-X259D, X0225N-X161G-X168T-X259E, X0225N-X161G-X168T-X261D, X0225N-X161G-X168T-X261E, X0225N-X161G-X168T-X276W, X0225N-X161G-X172F-X259D, X0225N-X161G-X172F-X259E, X0225N-X161G-X172F-X261D, X0225N-X161G-X172F-X261E, X0225N-X161G-X172F-X276W, X0225N-X161G-X259D-X261D, X0225N-X161G-X259D-X261E, X0225N-X161G-X259D-X276W, X0225N-X161G-X259E-X261D, X0225N-X161G-X259E-X261E, X0225N-X161G-X259E-X276W, X0225N-X161G-X261D-X276W, X0225N-X161G-X261E-X276W, X0225N-X168T-X172F-X259D, X0225N-X168T-X172F-X259E, X0225N-X168T-X172F-X261D, X0225N-X168T-X172F-X261E, X0225N-X168T-X172F-X276W, X0225N-X168T-X259D-X261D, X0225N-X168T-X259D-X261E, X0225N-X168T-X259D-X276W, X0225N-X168T-X259E-X261D, X0225N-X168T-X259E-X261E, X0225N-X168T-X259E-X276W, X0225N-X168T-X261D-X276W, X0225N-X168T-X261E-X276W, X0225N-X172F-X259D-X261D, X0225N-X172F-X259D-X261E, X0225N-X172F-X259D-X276W, X0225N-X172F-X259E-X261D, X0225N-X172F-X259E-X261E, X0225N-X172F-X259E-X276W, X0225N-X172F-X261D-X276W, X0225N-X172F-X261E-X276W, X0225N-X259D-X261D-X276W, X0225N-X259D-X261E-X276W, X0225N-X259E-X261D-X276W, X0225N-X259E-X261E-X276W, X032Y-X072V-X093Q-X131S, X032Y-X072V-X093Q-X139R, X032Y-X072V-X093Q-X161G, X032Y-X072V-X093Q-X168T, X032Y-X072V-X093Q-X172F, X032Y-X072V-X093Q-X225Q, X032Y-X072V-X093Q-X259D, X032Y-X072V-X093Q-X259E, X032Y-X072V-X093Q-X261D, X032Y-X072V-X093Q-X261E, X032Y-X072V-X093Q-X276W, X032Y-X072V-X131S-X139R, X032Y-X072V-X131S-X161G, X032Y-X072V-X131S-X168T, X032Y-X072V-X131S-X172F, X032Y-X072V-X131S-X225Q, X032Y-X072V-X131S-X259D, X032Y-X072V-X131S-X259E, X032Y-X072V-X131S-X261D, X032Y-X072V-X131S-X261E, X032Y-X072V-X131S-X276W, X032Y-X072V-X139R-X161G, X032Y-X072V-X139R-X168T, X032Y-X072V-X139R-X172F, X032Y-X072V-X139R-X225Q, X032Y-X072V-X139R-X259D, X032Y-X072V-X139R-X259E, X032Y-X072V-X139R-X261D, X032Y-X072V-X139R-X261E, X032Y-X072V-X139R-X276W, X032Y-X072V-X161G-X168T, X032Y-X072V-X161G-X172F, X032Y-X072V-X161G-X225Q, X032Y-X072V-X161G-X259D, X032Y-X072V-X161G-X259E, X032Y-X072V-X161G-X261D, X032Y-X072V-X161G-X261E, X032Y-X072V-X161G-X276W, X032Y-X072V-X168T-X172F, X032Y-X072V-X168T-X225Q, X032Y-X072V-X168T-X259D, X032Y-X072V-X168T-X259E, X032Y-X072V-X168T-X261D, X032Y-X072V-X168T-X261E, X032Y-X072V-X168T-X276W, X032Y-X072V-X172F-X225Q, X032Y-X072V-X172F-X259D, X032Y-X072V-X172F-X259E, X032Y-X072V-X172F-X261D, X032Y-X072V-X172F-X261E, X032Y-X072V-X172F-X276W, X032Y-X072V-X225Q-X259D, X032Y-X072V-X225Q-X259E, X032Y-X072V-X225Q-X261D, X032Y-X072V-X225Q-X261E, X032Y-X072V-X225Q-X276W, X032Y-X072V-X259D-X261D, X032Y-X072V-X259D-X261E, X032Y-X072V-X259D-X276W, X032Y-X072V-X259E-X261D, X032Y-X072V-X259E-X261E, X032Y-X072V-X259E-X276W, X032Y-X072V-X261D-X276W, X032Y-X072V-X261E-X276W, X032Y-X093Q-X131S-X139R, X032Y-X093Q-X131S-X161G, X032Y-X093Q-X131S-X168T, X032Y-X093Q-X131S-X172F, X032Y-X093Q-X131S-X225Q, X032Y-X093Q-X131S-X259D, X032Y-X093Q-X131S-X259E, X032Y-X093Q-X131S-X261D, X032Y-X093Q-X131S-X261E, X032Y-X093Q-X131S-X276W, X032Y-X093Q-X139R-X161G, X032Y-X093Q-X139R-X168T, X032Y-X093Q-X139R-X172F, X032Y-X093Q-X139R-X225Q, X032Y-X093Q-X139R-X259D, X032Y-X093Q-X139R-X259E, X032Y-X093Q-X139R-X261D, X032Y-X093Q-X139R-X261E, X032Y-X093Q-X139R-X276W, X032Y-X093Q-X161G-X168T, X032Y-X093Q-X161G-X172F, X032Y-X093Q-X161G-X225Q, X032Y-X093Q-X161G-X259D, X032Y-X093Q-X161G-X259E, X032Y-X093Q-X161G-X261D, X032Y-X093Q-X161G-X261E, X032Y-X093Q-X161G-X276W, X032Y-X093Q-X168T-X172F, X032Y-X093Q-X168T-X225Q, X032Y-X093Q-X168T-X259D, X032Y-X093Q-X168T-X259E, X032Y-X093Q-X168T-X261D, X032Y-X093Q-X168T-X261E, X032Y-X093Q-X168T-X276W, X032Y-X093Q-X172F-X225Q, X032Y-X093Q-X172F-X259D, X032Y-X093Q-X172F-X259E, X032Y-X093Q-X172F-X261D, X032Y-X093Q-X172F-X261E, X032Y-X093Q-X172F-X276W, X032Y-X093Q-X225Q-X259D, X032Y-X093Q-X225Q-X259E, X032Y-X093Q-X225Q-X261D, X032Y-X093Q-X225Q-X261E, X032Y-X093Q-X225Q-X276W, X032Y-X093Q-X259D-X261D, X032Y-X093Q-X259D-X261E, X032Y-X093Q-X259D-X276W, X032Y-X093Q-X259E-X261D, X032Y-X093Q-X259E-X261E, X032Y-X093Q-X259E-X276W, X032Y-X093Q-X261D-X276W, X032Y-X093Q-X261E-X276W, X032Y-X131S-X139R-X161G, X032Y-X131S-X139R-X168T, X032Y-X131S-X139R-X172F, X032Y-X131S-X139R-X225Q, X032Y-X131S-X139R-X259D, X032Y-X131S-X139R-X259E, X032Y-X131S-X139R-X261D, X032Y-X131S-X139R-X261E, X032Y-X131S-X139R-X276W, X032Y-X131S-X161G-X168T, X032Y-X131S-X161G-X172F, X032Y-X131S-X161G-X225Q, X032Y-X131S-X161G-X259D, X032Y-X131S-X161G-X259E, X032Y-X131S-X161G-X261D, X032Y-X131S-X161G-X261E, X032Y-X131S-X161G-X276W, X032Y-X131S-X168T-X172F, X032Y-X131S-X168T-X225Q, X032Y-X131S-X168T-X259D, X032Y-X131S-X168T-X259E, X032Y-X131S-X168T-X261D, X032Y-X131S-X168T-X261E, X032Y-X131S-X168T-X276W, X032Y-X131S-X172F-X225Q, X032Y-X131S-X172F-X259D, X032Y-X131S-X172F-X259E, X032Y-X131S-X172F-X261D, X032Y-X131S-X172F-X261E, X032Y-X131S-X172F-X276W, X032Y-X131S-X225Q-X259D, X032Y-X131S-X225Q-X259E, X032Y-X131S-X225Q-X261D, X032Y-X131S-X225Q-X261E, X032Y-X131S-X225Q-X276W, X032Y-X131S-X259D-X261D, X032Y-X131S-X259D-X261E, X032Y-X131S-X259D-X276W, X032Y-X131S-X259E-X261D, X032Y-X131S-X259E-X261E, X032Y-X131S-X259E-X276W, X032Y-X131S-X261D-X276W, X032Y-X131S-X261E-X276W, X032Y-X139R-X161G-X168T, X032Y-X139R-X161G-X172F, X032Y-X139R-X161G-X225Q, X032Y-X139R-X161G-X259D, X032Y-X139R-X161G-X259E, X032Y-X139R-X161G-X261D, X032Y-X139R-X161G-X261E, X032Y-X139R-X161G-X276W, X032Y-X139R-X168T-X172F, X032Y-X139R-X168T-X225Q, X032Y-X139R-X168T-X259D, X032Y-X139R-X168T-X259E, X032Y-X139R-X168T-X261D, X032Y-X139R-X168T-X261E, X032Y-X139R-X168T-X276W, X032Y-X139R-X172F-X225Q, X032Y-X139R-X172F-X259D, X032Y-X139R-X172F-X259E, X032Y-X139R-X172F-X261D, X032Y-X139R-X172F-X261E, X032Y-X139R-X172F-X276W, X032Y-X139R-X225Q-X259D, X032Y-X139R-X225Q-X259E, X032Y-X139R-X225Q-X261D, X032Y-X139R-X225Q-X261E, X032Y-X139R-X225Q-X276W, X032Y-X139R-X259D-X261D, X032Y-X139R-X259D-X261E, X032Y-X139R-X259D-X276W, X032Y-X139R-X259E-X261D, X032Y-X139R-X259E-X261E, X032Y-X139R-X259E-X276W, X032Y-X139R-X261D-X276W, X032Y-X139R-X261E-X276W, X032Y-X161G-X168T-X172F, X032Y-X161G-X168T-X225Q, X032Y-X161G-X168T-X259D, X032Y-X161G-X168T-X259E, X032Y-X161G-X168T-X261D, X032Y-X161G-X168T-X261E, X032Y-X161G-X168T-X276W, X032Y-X161G-X172F-X225Q, X032Y-X161G-X172F-X259D, X032Y-X161G-X172F-X259E, X032Y-X161G-X172F-X261D, X032Y-X161G-X172F-X261E, X032Y-X161G-X172F-X276W, X032Y-X161G-X225Q-X259D, X032Y-X161G-X225Q-X259E, X032Y-X161G-X225Q-X261D, X032Y-X161G-X225Q-X261E, X032Y-X161G-X225Q-X276W, X032Y-X161G-X259D-X261D, X032Y-X161G-X259D-X261E, X032Y-X161G-X259D-X276W, X032Y-X161G-X259E-X261D, X032Y-X161G-X259E-X261E, X032Y-X161G-X259E-X276W, X032Y-X161G-X261D-X276W, X032Y-X161G-X261E-X276W, X032Y-X168T-X172F-X225Q, X032Y-X168T-X172F-X259D, X032Y-X168T-X172F-X259E, X032Y-X168T-X172F-X261D, X032Y-X168T-X172F-X261E, X032Y-X168T-X172F-X276W, X032Y-X168T-X225Q-X259D, X032Y-X168T-X225Q-X259E, X032Y-X168T-X225Q-X261D, X032Y-X168T-X225Q-X261E, X032Y-X168T-X225Q-X276W, X032Y-X168T-X259D-X261D, X032Y-X168T-X259D-X261E, X032Y-X168T-X259D-X276W, X032Y-X168T-X259E-X261D, X032Y-X168T-X259E-X261E, X032Y-X168T-X259E-X276W, X032Y-X168T-X261D-X276W, X032Y-X168T-X261E-X276W, X032Y-X172F-X225Q-X259D, X032Y-X172F-X225Q-X259E, X032Y-X172F-X225Q-X261D, X032Y-X172F-X225Q-X261E, X032Y-X172F-X225Q-X276W, X032Y-X172F-X259D-X261D, X032Y-X172F-X259D-X261E, X032Y-X172F-X259D-X276W, X032Y-X172F-X259E-X261D, X032Y-X172F-X259E-X261E, X032Y-X172F-X259E-X276W, X032Y-X172F-X261D-X276W, X032Y-X172F-X261E-X276W, X032Y-X225Q-X259D-X261D, X032Y-X225Q-X259D-X261E, X032Y-X225Q-X259D-X276W, X032Y-X225Q-X259E-X261D, X032Y-X225Q-X259E-X261E, X032Y-X225Q-X259E-X276W, X032Y-X225Q-X261D-X276W, X032Y-X225Q-X261E-X276W, X032Y-X259D-X261D-X276W, X032Y-X259D-X261E-X276W, X032Y-X259E-X261D-X276W, X032Y-X259E-X261E-X276W, X072V-X093Q-X131S-X139R, X072V-X093Q-X131S-X161G, X072V-X093Q-X131S-X168T, X072V-X093Q-X131S-X172F, X072V-X093Q-X131S-X225Q, X072V-X093Q-X131S-X259D, X072V-X093Q-X131S-X259E, X072V-X093Q-X131S-X261D, X072V-X093Q-X131S-X261E, X072V-X093Q-X131S-X276W, X072V-X093Q-X139R-X161G, X072V-X093Q-X139R-X168T, X072V-X093Q-X139R-X172F, X072V-X093Q-X139R-X225Q, X072V-X093Q-X139R-X259D, X072V-X093Q-X139R-X259E, X072V-X093Q-X139R-X261D, X072V-X093Q-X139R-X261E, X072V-X093Q-X139R-X276W, X072V-X093Q-X161G-X168T, X072V-X093Q-X161G-X172F, X072V-X093Q-X161G-X225Q, X072V-X093Q-X161G-X259D, X072V-X093Q-X161G-X259E, X072V-X093Q-X161G-X261D, X072V-X093Q-X161G-X261E, X072V-X093Q-X161G-X276W, X072V-X093Q-X168T-X172F, X072V-X093Q-X168T-X225Q, X072V-X093Q-X168T-X259D, X072V-X093Q-X168T-X259E, X072V-X093Q-X168T-X261D, X072V-X093Q-X168T-X261E, X072V-X093Q-X168T-X276W, X072V-X093Q-X172F-X225Q, X072V-X093Q-X172F-X259D, X072V-X093Q-X172F-X259E, X072V-X093Q-X172F-X261D, X072V-X093Q-X172F-X261E, X072V-X093Q-X172F-X276W, X072V-X093Q-X225Q-X259D, X072V-X093Q-X225Q-X259E, X072V-X093Q-X225Q-X261D, X072V-X093Q-X225Q-X261E, X072V-X093Q-X225Q-X276W, X072V-X093Q-X259D-X261D, X072V-X093Q-X259D-X261E, X072V-X093Q-X259D-X276W, X072V-X093Q-X259E-X261D, X072V-X093Q-X259E-X261E, X072V-X093Q-X259E-X276W, X072V-X093Q-X261D-X276W, X072V-X093Q-X261E-X276W, X072V-X131S-X139R-X161G, X072V-X131S-X139R-X168T, X072V-X131S-X139R-X172F, X072V-X131S-X139R-X225Q, X072V-X131S-X139R-X259D, X072V-X131S-X139R-X259E, X072V-X131S-X139R-X261D, X072V-X131S-X139R-X261E, X072V-X131S-X139R-X276W, X072V-X131S-X161G-X168T, X072V-X131S-X161G-X172F, X072V-X131S-X161G-X225Q, X072V-X131S-X161G-X259D, X072V-X131S-X161G-X259E, X072V-X131S-X161G-X261D, X072V-X131S-X161G-X261E, X072V-X131S-X161G-X276W, X072V-X131S-X168T-X172F, X072V-X131S-X168T-X225Q, X072V-X131S-X168T-X259D, X072V-X131S-X168T-X259E, X072V-X131S-X168T-X261D, X072V-X131S-X168T-X261E, X072V-X131S-X168T-X276W, X072V-X131S-X172F-X225Q, X072V-X131S-X172F-X259D, X072V-X131S-X172F-X259E, X072V-X131S-X172F-X261D, X072V-X131S-X172F-X261E, X072V-X131S-X172F-X276W, X072V-X131S-X225Q-X259D, X072V-X131S-X225Q-X259E, X072V-X131S-X225Q-X261D, X072V-X131S-X225Q-X261E, X072V-X131S-X225Q-X276W, X072V-X131S-X259D-X261D, X072V-X131S-X259D-X261E, X072V-X131S-X259D-X276W, X072V-X131S-X259E-X261D, X072V-X131S-X259E-X261E, X072V-X131S-X259E-X276W, X072V-X131S-X261D-X276W, X072V-X131S-X261E-X276W, X072V-X139R-X161G-X168T, X072V-X139R-X161G-X172F, X072V-X139R-X161G-X225Q, X072V-X139R-X161G-X259D, X072V-X139R-X161G-X259E, X072V-X139R-X161G-X261D, X072V-X139R-X161G-X261E, X072V-X139R-X161G-X276W, X072V-X139R-X168T-X172F, X072V-X139R-X168T-X225Q, X072V-X139R-X168T-X259D, X072V-X139R-X168T-X259E, X072V-X139R-X168T-X261D, X072V-X139R-X168T-X261E, X072V-X139R-X168T-X276W, X072V-X139R-X172F-X225Q, X072V-X139R-X172F-X259D, X072V-X139R-X172F-X259E, X072V-X139R-X172F-X261D, X072V-X139R-X172F-X261E, X072V-X139R-X172F-X276W, X072V-X139R-X225Q-X259D, X072V-X139R-X225Q-X259E, X072V-X139R-X225Q-X261D, X072V-X139R-X225Q-X261E, X072V-X139R-X225Q-X276W, X072V-X139R-X259D-X261D, X072V-X139R-X259D-X261E, X072V-X139R-X259D-X276W, X072V-X139R-X259E-X261D, X072V-X139R-X259E-X261E, X072V-X139R-X259E-X276W, X072V-X139R-X261D-X276W, X072V-X139R-X261E-X276W, X072V-X161G-X168T-X172F, X072V-X161G-X168T-X225Q, X072V-X161G-X168T-X259D, X072V-X161G-X168T-X259E, X072V-X161G-X168T-X261D, X072V-X161G-X168T-X261E, X072V-X161G-X168T-X276W, X072V-X161G-X172F-X225Q, X072V-X161G-X172F-X259D, X072V-X161G-X172F-X259E, X072V-X161G-X172F-X261D, X072V-X161G-X172F-X261E, X072V-X161G-X172F-X276W, X072V-X161G-X225Q-X259D, X072V-X161G-X225Q-X259E, X072V-X161G-X225Q-X261D, X072V-X161G-X225Q-X261E, X072V-X161G-X225Q-X276W, X072V-X161G-X259D-X261D, X072V-X161G-X259D-X261E, X072V-X161G-X259D-X276W, X072V-X161G-X259E-X261D, X072V-X161G-X259E-X261E, X072V-X161G-X259E-X276W, X072V-X161G-X261D-X276W, X072V-X161G-X261E-X276W, X072V-X168T-X172F-X225Q, X072V-X168T-X172F-X259D, X072V-X168T-X172F-X259E, X072V-X168T-X172F-X261D, X072V-X168T-X172F-X261E, X072V-X168T-X172F-X276W, X072V-X168T-X225Q-X259D, X072V-X168T-X225Q-X259E, X072V-X168T-X225Q-X261D, X072V-X168T-X225Q-X261E, X072V-X168T-X225Q-X276W, X072V-X168T-X259D-X261D, X072V-X168T-X259D-X261E, X072V-X168T-X259D-X276W, X072V-X168T-X259E-X261D, X072V-X168T-X259E-X261E, X072V-X168T-X259E-X276W, X072V-X168T-X261D-X276W, X072V-X168T-X261E-X276W, X072V-X172F-X225Q-X259D, X072V-X172F-X225Q-X259E, X072V-X172F-X225Q-X261D, X072V-X172F-X225Q-X261E, X072V-X172F-X225Q-X276W, X072V-X172F-X259D-X261D, X072V-X172F-X259D-X261E, X072V-X172F-X259D-X276W, X072V-X172F-X259E-X261D, X072V-X172F-X259E-X261E, X072V-X172F-X259E-X276W, X072V-X172F-X261D-X276W, X072V-X172F-X261E-X276W, X072V-X225Q-X259D-X261D, X072V-X225Q-X259D-X261E, X072V-X225Q-X259D-X276W, X072V-X225Q-X259E-X261D, X072V-X225Q-X259E-X261E, X072V-X225Q-X259E-X276W, X072V-X225Q-X261D-X276W, X072V-X225Q-X261E-X276W, X072V-X259D-X261D-X276W, X072V-X259D-X261E-X276W, X072V-X259E-X261D-X276W, X072V-X259E-X261E-X276W, X093Q-X131S-X139R-X161G, X093Q-X131S-X139R-X168T, X093Q-X131S-X139R-X172F, X093Q-X131S-X139R-X225Q, X093Q-X131S-X139R-X259D, X093Q-X131S-X139R-X259E, X093Q-X131S-X139R-X261D, X093Q-X131S-X139R-X261E, X093Q-X131S-X139R-X276W, X093Q-X131S-X161G-X168T, X093Q-X131S-X161G-X172F, X093Q-X131S-X161G-X225Q, X093Q-X131S-X161G-X259D, X093Q-X131S-X161G-X259E, X093Q-X131S-X161G-X261D, X093Q-X131S-X161G-X261E, X093Q-X131S-X161G-X276W, X093Q-X131S-X168T-X172F, X093Q-X131S-X168T-X225Q, X093Q-X131S-X168T-X259D, X093Q-X131S-X168T-X259E, X093Q-X131S-X168T-X261D, X093Q-X131S-X168T-X261E, X093Q-X131S-X168T-X276W, X093Q-X131S-X172F-X225Q, X093Q-X131S-X172F-X259D, X093Q-X131S-X172F-X259E, X093Q-X131S-X172F-X261D, X093Q-X131S-X172F-X261E, X093Q-X131S-X172F-X276W, X093Q-X131S-X225Q-X259D, X093Q-X131S-X225Q-X259E, X093Q-X131S-X225Q-X261D, X093Q-X131S-X225Q-X261E, X093Q-X131S-X225Q-X276W, X093Q-X131S-X259D-X261D, X093Q-X131S-X259D-X261E, X093Q-X131S-X259D-X276W, X093Q-X131S-X259E-X261D, X093Q-X131S-X259E-X261E, X093Q-X131S-X259E-X276W, X093Q-X131S-X261D-X276W, X093Q-X131S-X261E-X276W, X093Q-X139R-X161G-X168T, X093Q-X139R-X161G-X172F, X093Q-X139R-X161G-X225Q, X093Q-X139R-X161G-X259D, X093Q-X139R-X161G-X259E, X093Q-X139R-X161G-X261D, X093Q-X139R-X161G-X261E, X093Q-X139R-X161G-X276W, X093Q-X139R-X168T-X172F, X093Q-X139R-X168T-X225Q, X093Q-X139R-X168T-X259D, X093Q-X139R-X168T-X259E, X093Q-X139R-X168T-X261D, X093Q-X139R-X168T-X261E, X093Q-X139R-X168T-X276W, X093Q-X139R-X172F-X225Q, X093Q-X139R-X172F-X259D, X093Q-X139R-X172F-X259E, X093Q-X139R-X172F-X261D, X093Q-X139R-X172F-X261E, X093Q-X139R-X172F-X276W, X093Q-X139R-X225Q-X259D, X093Q-X139R-X225Q-X259E, X093Q-X139R-X225Q-X261D, X093Q-X139R-X225Q-X261E, X093Q-X139R-X225Q-X276W, X093Q-X139R-X259D-X261D, X093Q-X139R-X259D-X261E, X093Q-X139R-X259D-X276W, X093Q-X139R-X259E-X261D, X093Q-X139R-X259E-X261E, X093Q-X139R-X259E-X276W, X093Q-X139R-X261D-X276W, X093Q-X139R-X261E-X276W, X093Q-X161G-X168T-X172F, X093Q-X161G-X168T-X225Q, X093Q-X161G-X168T-X259D, X093Q-X161G-X168T-X259E, X093Q-X161G-X168T-X261D, X093Q-X161G-X168T-X261E, X093Q-X161G-X168T-X276W, X093Q-X161G-X172F-X225Q, X093Q-X161G-X172F-X259D, X093Q-X161G-X172F-X259E, X093Q-X161G-X172F-X261D, X093Q-X161G-X172F-X261E, X093Q-X161G-X172F-X276W, X093Q-X161G-X225Q-X259D, X093Q-X161G-X225Q-X259E, X093Q-X161G-X225Q-X261D, X093Q-X161G-X225Q-X261E, X093Q-X161G-X225Q-X276W, X093Q-X161G-X259D-X261D, X093Q-X161G-X259D-X261E, X093Q-X161G-X259D-X276W, X093Q-X161G-X259E-X261D, X093Q-X161G-X259E-X261E, X093Q-X161G-X259E-X276W, X093Q-X161G-X261D-X276W, X093Q-X161G-X261E-X276W, X093Q-X168T-X172F-X225Q, X093Q-X168T-X172F-X259D, X093Q-X168T-X172F-X259E, X093Q-X168T-X172F-X261D, X093Q-X168T-X172F-X261E, X093Q-X168T-X172F-X276W, X093Q-X168T-X225Q-X259D, X093Q-X168T-X225Q-X259E, X093Q-X168T-X225Q-X261D, X093Q-X168T-X225Q-X261E, X093Q-X168T-X225Q-X276W, X093Q-X168T-X259D-X261D, X093Q-X168T-X259D-X261E, X093Q-X168T-X259D-X276W, X093Q-X168T-X259E-X261D, X093Q-X168T-X259E-X261E, X093Q-X168T-X259E-X276W, X093Q-X168T-X261D-X276W, X093Q-X168T-X261E-X276W, X093Q-X172F-X225Q-X259D, X093Q-X172F-X225Q-X259E, X093Q-X172F-X225Q-X261D, X093Q-X172F-X225Q-X261E, X093Q-X172F-X225Q-X276W, X093Q-X172F-X259D-X261D, X093Q-X172F-X259D-X261E, X093Q-X172F-X259D-X276W, X093Q-X172F-X259E-X261D, X093Q-X172F-X259E-X261E, X093Q-X172F-X259E-X276W, X093Q-X172F-X261D-X276W, X093Q-X172F-X261E-X276W, X093Q-X225Q-X259D-X261D, X093Q-X225Q-X259D-X261E, X093Q-X225Q-X259D-X276W, X093Q-X225Q-X259E-X261D, X093Q-X225Q-X259E-X261E, X093Q-X225Q-X259E-X276W, X093Q-X225Q-X261D-X276W, X093Q-X225Q-X261E-X276W, X093Q-X259D-X261D-X276W, X093Q-X259D-X261E-X276W, X093Q-X259E-X261D-X276W, X093Q-X259E-X261E-X276W, X131S-X139R-X161G-X168T, X131S-X139R-X161G-X172F, X131S-X139R-X161G-X225Q, X131S-X139R-X161G-X259D, X131S-X139R-X161G-X259E, X131S-X139R-X161G-X261D, X131S-X139R-X161G-X261E, X131S-X139R-X161G-X276W, X131S-X139R-X168T-X172F, X131S-X139R-X168T-X225Q, X131S-X139R-X168T-X259D, X131S-X139R-X168T-X259E, X131S-X139R-X168T-X261D, X131S-X139R-X168T-X261E, X131S-X139R-X168T-X276W, X131S-X139R-X172F-X225Q, X131S-X139R-X172F-X259D, X131S-X139R-X172F-X259E, X131S-X139R-X172F-X261D, X131S-X139R-X172F-X261E, X131S-X139R-X172F-X276W, X131S-X139R-X225Q-X259D, X131S-X139R-X225Q-X259E, X131S-X139R-X225Q-X261D, X131S-X139R-X225Q-X261E, X131S-X139R-X225Q-X276W, X131S-X139R-X259D-X261D, X131S-X139R-X259D-X261E, X131S-X139R-X259D-X276W, X131S-X139R-X259E-X261D, X131S-X139R-X259E-X261E, X131S-X139R-X259E-X276W, X131S-X139R-X261D-X276W, X131S-X139R-X261E-X276W, X131S-X161G-X168T-X172F, X131S-X161G-X168T-X225Q, X131S-X161G-X168T-X259D, X131S-X161G-X168T-X259E, X131S-X161G-X168T-X261D, X131S-X161G-X168T-X261E, X131S-X161G-X168T-X276W, X131S-X161G-X172F-X225Q, X131S-X161G-X172F-X259D, X131S-X161G-X172F-X259E, X131S-X161G-X172F-X261D, X131S-X161G-X172F-X261E, X131S-X161G-X172F-X276W, X131S-X161G-X225Q-X259D, X131S-X161G-X225Q-X259E, X131S-X161G-X225Q-X261D, X131S-X161G-X225Q-X261E, X131S-X161G-X225Q-X276W, X131S-X161G-X259D-X261D, X131S-X161G-X259D-X261E, X131S-X161G-X259D-X276W, X131S-X161G-X259E-X261D, X131S-X161G-X259E-X261E, X131S-X161G-X259E-X276W, X131S-X161G-X261D-X276W, X131S-X161G-X261E-X276W, X131S-X168T-X172F-X225Q, X131S-X168T-X172F-X259D, X131S-X168T-X172F-X259E, X131S-X168T-X172F-X261D, X131S-X168T-X172F-X261E, X131S-X168T-X172F-X276W, X131S-X168T-X225Q-X259D, X131S-X168T-X225Q-X259E, X131S-X168T-X225Q-X261D, X131S-X168T-X225Q-X261E, X131S-X168T-X225Q-X276W, X131S-X168T-X259D-X261D, X131S-X168T-X259D-X261E, X131S-X168T-X259D-X276W, X131S-X168T-X259E-X261D, X131S-X168T-X259E-X261E, X131S-X168T-X259E-X276W, X131S-X168T-X261D-X276W, X131S-X168T-X261E-X276W, X131S-X172F-X225Q-X259D, X131S-X172F-X225Q-X259E, X131S-X172F-X225Q-X261D, X131S-X172F-X225Q-X261E, X131S-X172F-X225Q-X276W, X131S-X172F-X259D-X261D, X131S-X172F-X259D-X261E, X131S-X172F-X259D-X276W, X131S-X172F-X259E-X261D, X131S-X172F-X259E-X261E, X131S-X172F-X259E-X276W, X131S-X172F-X261D-X276W, X131S-X172F-X261E-X276W, X131S-X225Q-X259D-X261D, X131S-X225Q-X259D-X261E, X131S-X225Q-X259D-X276W, X131S-X225Q-X259E-X261D, X131S-X225Q-X259E-X261E, X131S-X225Q-X259E-X276W, X131S-X225Q-X261D-X276W, X131S-X225Q-X261E-X276W, X131S-X259D-X261D-X276W, X131S-X259D-X261E-X276W, X131S-X259E-X261D-X276W, X131S-X259E-X261E-X276W, X139R-X161G-X168T-X172F, X139R-X161G-X168T-X225Q, X139R-X161G-X168T-X259D, X139R-X161G-X168T-X259E, X139R-X161G-X168T-X261D, X139R-X161G-X168T-X261E, X139R-X161G-X168T-X276W, X139R-X161G-X172F-X225Q, X139R-X161G-X172F-X259D, X139R-X161G-X172F-X259E, X139R-X161G-X172F-X261D, X139R-X161G-X172F-X261E, X139R-X161G-X172F-X276W, X139R-X161G-X225Q-X259D, X139R-X161G-X225Q-X259E, X139R-X161G-X225Q-X261D, X139R-X161G-X225Q-X261E, X139R-X161G-X225Q-X276W, X139R-X161G-X259D-X261D, X139R-X161G-X259D-X261E, X139R-X161G-X259D-X276W, X139R-X161G-X259E-X261D, X139R-X161G-X259E-X261E, X139R-X161G-X259E-X276W, X139R-X161G-X261D-X276W, X139R-X161G-X261E-X276W, X139R-X168T-X172F-X225Q, X139R-X168T-X172F-X259D, X139R-X168T-X172F-X259E, X139R-X168T-X172F-X261D, X139R-X168T-X172F-X261E, X139R-X168T-X172F-X276W, X139R-X168T-X225Q-X259D, X139R-X168T-X225Q-X259E, X139R-X168T-X225Q-X261D, X139R-X168T-X225Q-X261E, X139R-X168T-X225Q-X276W, X139R-X168T-X259D-X261D, X139R-X168T-X259D-X261E, X139R-X168T-X259D-X276W, X139R-X168T-X259E-X261D, X139R-X168T-X259E-X261E, X139R-X168T-X259E-X276W, X139R-X168T-X261D-X276W, X139R-X168T-X261E-X276W, X139R-X172F-X225Q-X259D, X139R-X172F-X225Q-X259E, X139R-X172F-X225Q-X261D, X139R-X172F-X225Q-X261E, X139R-X172F-X225Q-X276W, X139R-X172F-X259D-X261D, X139R-X172F-X259D-X261E, X139R-X172F-X259D-X276W, X139R-X172F-X259E-X261D, X139R-X172F-X259E-X261E, X139R-X172F-X259E-X276W, X139R-X172F-X261D-X276W, X139R-X172F-X261E-X276W, X139R-X225Q-X259D-X261D, X139R-X225Q-X259D-X261E, X139R-X225Q-X259D-X276W, X139R-X225Q-X259E-X261D, X139R-X225Q-X259E-X261E, X139R-X225Q-X259E-X276W, X139R-X225Q-X261D-X276W, X139R-X225Q-X261E-X276W, X139R-X259D-X261D-X276W, X139R-X259D-X261E-X276W, X139R-X259E-X261D-X276W, X139R-X259E-X261E-X276W, X161G-X168T-X172F-X225Q, X161G-X168T-X172F-X259D, X161G-X168T-X172F-X259E, X161G-X168T-X172F-X261D, X161G-X168T-X172F-X261E, X161G-X168T-X172F-X276W, X161G-X168T-X225Q-X259D, X161G-X168T-X225Q-X259E, X161G-X168T-X225Q-X261D, X161G-X168T-X225Q-X261E, X161G-X168T-X225Q-X276W, X161G-X168T-X259D-X261D, X161G-X168T-X259D-X261E, X161G-X168T-X259D-X276W, X161G-X168T-X259E-X261D, X161G-X168T-X259E-X261E, X161G-X168T-X259E-X276WX161G-X168T-X261D-X276W, X161G-X168T-X261E-X276W, X161G-X172F-X225Q-X259D, X161G-X172F-X225Q-X259E, X161G-X172F-X225Q-X261D, X161G-X172F-X225Q-X261E, X161G-X172F-X225Q-X276W, X161G-X172F-X259D-X261D, X161G-X172F-X259D-X261E, X161G-X172F-X259D-X276W, X161G-X172F-X259E-X261D, X161G-X172F-X259E-X261E, X161G-X172F-X259E-X276W, X161G-X172F-X261D-X276W, X161G-X172F-X261E-X276W, X161G-X225Q-X259D-X261D, X161G-X225Q-X259D-X261E, X161G-X225Q-X259D-X276W, X161G-X225Q-X259E-X261D, X161G-X225Q-X259E-X261E, X161G-X225Q-X259E-X276W, X161G-X225Q-X261D-X276W, X161G-X225Q-X261E-X276W, X161G-X259D-X261D-X276W, X161G-X259D-X261E-X276W, X161G-X259E-X261D-X276W, X161G-X259E-X261E-X276W, X168T-X172F-X225Q-X259D, X168T-X172F-X225Q-X259E, X168T-X172F-X225Q-X261D, X168T-X172F-X225Q-X261E, X168T-X172F-X225Q-X276W, X168T-X172F-X259D-X261D, X168T-X172F-X259D-X261E, X168T-X172F-X259D-X276W, X168T-X172F-X259E-X261D, X168T-X172F-X259E-X261E, X168T-X172F-X259E-X276W, X168T-X172F-X261D-X276W, X168T-X172F-X261E-X276W, X168T-X225Q-X259D-X261D, X168T-X225Q-X259D-X261E, X168T-X225Q-X259D-X276W, X168T-X225Q-X259E-X261D, X168T-X225Q-X259E-X261E, X168T-X225Q-X259E-X276W, X168T-X225Q-X261D-X276W, X168T-X225Q-X261E-X276W X168T-X259D-X261D-X276W, X168T-X259D-X261E-X276W, X168T-X259E-X261D-X276W, X168T-X259E-X261E-X276W, X172F-X225Q-X259D-X261D, X172F-X225Q-X259D-X261E, X172F-X225Q-X259D-X276W, X172F-X225Q-X259E-X261D, X172F-X225Q-X259E-X261E, X172F-X225Q-X259E-X276W, X172F-X225Q-X261D-X276W, X172F-X225Q-X261E-X276W, X172F-X259D-X261D-X276W, X172F-X259D-X261E-X276W, X172F-X259E-X261D-X276W, X172F-X259E-X261E-X276W, X225Q-X259D-X261D-X276W, X225Q-X259D-X261E-X276W, X225Q-X259E-X261D-X276W and X225Q-X259E-X261E-X276W.

### EXAMPLES

It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

### Example 1

### Assays

### Protein Determination Assays

HPLC detection and quantitation-Protein concentration determination was performed using a high-performance liquid chromatography (HPLC) method measuring integrated peak area. Samples were obtained from filtered culture supernatants and prepared as 3-fold dilutions in 10 mM HEPES buffer, pH 8. HPLC was carried out on an Agilent 1200 Series HPLC system equipped with a Poroshell 300SB-C8 column (2.1×75 mm) and Poroshell 300SB-C8 guard column (2.1×12.5 mm) using a gradient elution composed of water and acetonitrile solvents, each supplemented with 0.1% TFA. Samples were eluted at 65 °C, at a flow rate of 0.5 mL/min. Proteins were detected by measuring absorbance at 225 nm, and peaks were integrated using ChemStation OpenLab software (Agilent Technologies). The protein concentration of samples was determined based on a standard curve of a parent protein.

Bradford protein quantitation-Protein concentration measurement was performed using the Bradford assay (Thermo Scientific Coomassie Protein Assay Kit #23200). Samples were obtained from filtered culture supernatants and prepared as 3-fold dilutions in 10 mM HEPES buffer, pH 8. The protein concentration of samples was determined based on a standard curve of a parent protein. Background protein was subtracted utilizing the Bacillus subtilis host not expressing a recombinant mannanase.

### Mannanase Activity Assay

The mannanase activity was determined by measuring the hydrolysis of locust bean gum (LBG) galactomannan (Sigma-Aldrich, #G0753) substrate in solution (approximately 0.5% (w/v) LBG substrate). To prepare a working substrate solution, the LBG powder was dissolved in a heated solution of 50 mM Tris-HCl buffer, pH 7.5, under stirring. Upon cooling to room temperature, the solution was centrifuged, and the clear supernatant was used as the substrate solution. Enzymes were diluted into enzyme dilution buffer (100 mM HEPES, pH 8, 0.005% TWEEN®80) and aliquots of the diluted enzyme solutions were added to the wells of a microtiter plate (e.g. Corning 3358) containing the LBG substrate solution. The plates were sealed and incubated at 40 °C with agitation at 900 rpm for 10 min (e.g. in an iEMS incubator/shaker, Thermo Fisher). After incubation, the released reducing sugars were quantified using the BCA reagent assay (Catalog No. 23225, Thermo Scientific Pierce). Specifically, aliquots from each well of the LBG assay plate were added to a PCR plate containing BCA working reagent solution (prepared according to the manufacturer's instructions); the sample to working reagent ratio was 1:9 (v/v). The plates were sealed and incubated in a thermocycler (e.g. Applied Biosystems GeneAmp PCR System 9700) at 95 °C for 2.5 min. After the plate cooled to 25 °C, the reaction solution was transferred to a fresh flat-bottom clear polystyrene MTP (e.g. Falcon 353075) and absorbance was measured at 562 nm in a plate reader spectrophotometer (e.g. SpectraMax Plus 384, Molecular Devices). The absorbance value of a sample not containing mannanase (blank) was subtracted from the absorbance values of the mannanase-containing samples. The resulting absorbance was taken as a measure of mannanase activity.

### Detergents Used in Assays

Two detergents were used in the examples described below. Chinese National Standard HDL (CNS) was purchased from the China Cleaning Industry Association. The composition information of CNS is described in “Determination of detergency and cycle of washing property for laundry detergents”, National Standard of the People's republic of China, ICS 71.100.40, Y43 (GB/T 13174-2021), www.chinesestandard.net/PDF/BOOK.aspx/GBT13174-2021) and is shown on Table 2. Model HDL detergent formulation A composition is shown on Table 3.

TABLE 2
Composition of CNS HDL detergent.

| Raw Material | % w/w active matter |
|---|---|
| LAS | 8 |
| AES | 2 |
| AEO (EO = 9) | 4 |
| Triethanolamine | 0.5 |
| Sodium citrate | 0.5 |
| NaOH | Adjust pH to 8.5-9.0 |
| water | Up to 100 |

TABLE 3
Composition of Model HDL detergent formulation A.

| Raw material | % Active matter |
|---|---|
| Sodium Dodecylbenzene Sulfonate | 7.3 |
| Sodium Laureth Sulfate | 6.3 |
| Potassium Cocoate | 1.05 |
| Ethoxylated nonionic surfactant | 3.0 |
| Sodium Citrate | 2.65 |
| Ethanol | 1.0 |
| Propylene Glycol | 0.8 |
| Sorbitol | 0.8 |
| Triethanolamine | 0.5 |
| Nonionic polyester | 0.3 |
| Polycarboxylate | 1.38 |
| Polyvinylpyrrolidone | 0.08 |
| Methylisothiazolinone (2.5%) + | 0.01 |
| Benzisothiazolinone (2.5%) | |
| Adjust pH with NaOH or citric acid | 8.0-8.5 |
| Demin. Water | add to 100 |

### Stability Assays

The stability of the mannanase variants was tested under the stress condition described in the examples and by measuring the residual mannanase activity of samples after incubation under those conditions. To measure the initial (unstressed) activity, the enzyme samples were diluted in 100 mM HEPES, pH 8, 0.005% TWEEN®80 and added to the detergent condition as described in the Examples followed by immediate storage at −80 °C. To measure the stressed activity, the enzyme samples were diluted in 100 mM HEPES, pH 8, 0.005% TWEEN®80 and added to the detergent condition and incubated as described in the Examples followed by storage at −80 °C. Samples were then assayed for activity on LBG substrate using the assay described in the “Mannanase Activity Assay” section above. The percent (%) residual activities were calculated by taking a ratio of the stressed to unstressed activity and multiplying by 100.

### Cleaning Performance of Mannanase Variants Using Microswatch Assays

Mannanase samples were tested for cleaning performance on locust bean gum (LBG) microswatches (CFT C-S-73, Center for Testmaterials, Vlaardingen, The Netherlands), and the results for mannanase variants was reported relative to performance of a parent or reference enzyme. Cleaning performance was measured using a high throughput assay developed to measure galactomannan removal from technical soils. The assay measures the release of LBG from the technical soils containing LBG. The BCA reaction using a commercially available reagent (Catalog No. 23225, Thermo Scientific Pierce) was used to measure reducing ends of oligosaccharides in solution in the presence of enzyme, compared to a blank control. This measurement correlates with the cleaning performance of the enzyme. As the mannanase hydrolyzes galactomannans, oligosaccharides of varying lengths with reducing ends are presumably released from the cotton swatch. The bicinchoninic acid in the BCA reagent then allows for the highly sensitive colorimetric detection of Cu¹⁺ formed by the reduction of Cu²⁺. Two 5 mm diameter LBG microswatches were placed into each well of a flat-bottom 96-well assay plate. Enzymes were diluted with a buffer containing 10 mM NaCl, 0.1 mM CaCl2, and 0.005% (v/v) TWEEN®80. 90 μL microswatch assay buffer (6 g/L model HDL detergent formulation A, 5 mM HEPES pH 8.0, 200 ppm 3:1 Ca2+/Mg2+ water hardness) and 10 μL aliquots of diluted enzymes were added into each well of the 96-well microswatch assay plate for a combined volume of 100 μL. Plates were sealed and incubated at 25 °C with agitation at 1150 rpm for 20 min (e.g. in an iEMS incubator shaker, Thermo Fisher). After the incubation, the released reducing sugars were quantified using the BCA reagent assay as described in the “Mannanase Activity Assay” section above. The resulting absorbance was taken as a measure of cleaning performance.

The performance index (PI) of an enzyme compares the performance of the variant (measured value) with the parent or reference enzyme (theoretical value or measured value) tested at the same protein concentration. Theoretical values for the cleaning performance of the parent or reference enzyme at the relevant protein concentrations were calculated using the parameters extracted from a Langmuir fit of measured values for a standard curve of the parent or reference enzyme.

All mannanase variants described in this application show cleaning performance on par or superior to parent or reference enzyme.

### Example 2

### Variant and Strain Construction and Sample Preparation

Synthetic genes encoding mannanase variants based on Paenibacillus sp. A1 mannanase PspMan4 (SEQ ID NO: 1) were designed for expression in Bacillus subtilis. These genes were cloned and expressed in Bacillus subtilis using methods known in the art. To generate samples of mannanase variants for characterization, growth of the Bacillus subtilis strains was performed by standard methods in a cultivation medium (enriched semi-defined medium based on MOPS buffer, urea as the major nitrogen source, glucose as the main carbon source, and supplemented with soytone) and supernatants were filtered through a 0.2 μm filter plate (PALL). The filtered culture supernatants were used for the measurement of enzyme activity, stability and cleaning performance according to the assays described in Example 1.

### Example 3

### Evaluation of Stability of Mannanase Variants in Detergent Formulations

The stability of mannanase variants was tested using the method described in Example 1 Stability Assay under the stress conditions described for each table below where results are reported as percent (%) residual activity. The mutations of mannanase variants are reported as relative to parent enzyme, PspMan4 (SEQ ID NO: 1) or PspMan138 (SEQ ID NO: 2) as noted on data Tables. PspMan138 is a variant of PspMan4 with the following mutations: P019E-S030T-T038E-S059V-L060Q-K063R-N067D-N097D-V1031-Y129M-F167Y-Q184L-G225C-T228V-Y235L-K244L-S258D-N261R-ins298Q.

The stability of mannanase variants as compared to PspMan4 parent was tested in a 25% (v/v) aqueous solution of Model HDL detergent formulation A described in Example 1 using Stress Condition A (30 °C for 5 minutes). The stability results under these conditions are reported as residual activity using the calculation described in Example 1, and the values are reported in Table 4.

TABLE 4
Stability of PspMan4 Variants in model HDL detergent
formulation A under stress condition A.

| | | Condition A |
|---|---|---|
| | Mutations relative to | % Residual |
| Sample ID | PspMan4 | Activity |
| PspMan4 | None | 40% |
| TL0674 | F032Y | 60% |
| TL0678 | I072V | 60% |
| TL0694 | V172F | 50% |
| TL0698 | G259D | 90% |
| TL0699 | N261D | 70% |
| TL0700 | N261E | 90% |
| TL0705 | F276W | 100% |

The stability of mannanase variants as compared to PspMan138 parent was tested in a 25% (v/v) aqueous solution of Model HDL detergent formulation A described in Example 1 using Stress Condition B (55 °C incubation for 20 minutes) or Stress Condition C (55 °C incubation for 60 minutes). The residual activity results are reported in Table 5.

TABLE 5
Stability of PspMan138 Variants in model HDL detergent
formulation A under stress conditions B and C.

| | | % Residual Activity | |
|---|---|---|---|
| | Mutations relative | Condition | Condition |
| Sample ID | to PspMan138 | B | C |
| PspMan138 | None | 20% | 0% |
| TL0020 | F032Y | 30% | ND |
| TL0074 | T131S | 30% | 10% |
| TL0088 | V172F | 30% | ND |
| TL0145 | G259D | 90% | 40% |
| TL0148 | R261E | 100% | 40% |
| TL0150 | R261D | 60% | 10% |
| TL0158 | S275E | 60% | ND |
| TL0159 | F276W | 100% | 100% |
| TL0167 | K293L | 50% | ND |
| TL0174 | N034D | 40% | ND |
| TL0178 | S257E | 60% | ND |
| TL0179 | T284E | 40% | ND |
| TL0237 | K045E | 60% | 10% |
| TL0238 | T062E | 30% | ND |
| TL0273 | G259D-F276W | 100% | 100% |
| TL0274 | R261E-F276W | 100% | 100% |
| TL0275 | R261D-F276W | 100% | 100% |
| TL0276 | G259D-R261E-F276W | 100% | 100% |
| TL0317 | Y061W-R261E | ND | 30% |
| TL0319 | Y061W-G259D-R261E-F276W | ND | 100% |
| TL0320 | Y061W-T062E-G259D-R261E-F276W | ND | 100% |
| TL0321 | Y061W-T062E-R261E | ND | 60% |
| TL0324 | G259Q-R261E-F276W | ND | 100% |
| TL0331 | Y061W-V228T-G259D-R261E-F276W | ND | 100% |
| TL0332 | V228T-G259D-R261E-F276W | ND | 100% |
| TL0337 | F032Y-G259D-R261E-F276W | ND | 100% |
| TL0338 | F032Y-Y061W-Y167F-P168S-G259D- | ND | 100% |
| | R261E-F276W | | |
| TL0339 | F032Y-Y061W-G259D-R261E-F276W | ND | 100% |
| TL0340 | F032Y-Y061W-T062E-G259D-R261E- | ND | 100% |
| | F276W | | |
| TL0373 | T062E-F276W | ND | 100% |
| TL0374 | T062E-G259D | ND | 50% |
| TL0375 | T062E-R261D | ND | 20% |
| TL0376 | T062E-R261D-F276W | ND | 100% |
| TL0377 | T062E-G259D-F276W | ND | 100% |
| TL0381 | F032Y-R261D-F276W | ND | 100% |
| TL0382 | F032Y-G259D-F276W | ND | 100% |
| TL0385 | F032Y-G259D | ND | 50% |
| TL0391 | F032Y-T062E-R261D-F276W | ND | 100% |
| TL0392 | F032Y-T062E-G259D | ND | 50% |
| TL0393 | F032Y-T062E-G259D-F276W | ND | 100% |
| TL0605 | T062E-G259D-R261E-F276W | ND | 100% |
| TL0606 | V059S-Q060L-G259D-R261E-F276W | ND | 100% |
| TL0607 | V059S-Q060L-Y061W-G259D-R261E- | ND | 100% |
| | F276W | | |
| TL0608 | V059S-Q060L-T062E-G259D-R261E- | ND | 100% |
| | F276W | | |
| TL0612 | T062E-V228T-G259D-R261E-F276W | ND | 100% |
| TL0613 | F032Y-T062E-G259D-R261E-F276W | ND | 100% |
| TL0614 | F032Y-T062E-G259D-R261E-F276W- | ND | 100% |
| | T284E | | |
| TL0735 | R261E-T284E | ND | 70% |
| TL0743 | T062E-R261E | ND | 70% |
| TL0907 | F032Y-V172F-G259D | ND | 60% |
| TL0957 | T131S-F276W | ND | 100% |
| TL0987 | E019D-F276W | ND | 100% |
| TL0988 | E019D-A068S-F276W | ND | 100% |
| TL0989 | E019D-T131S-F276W | ND | 100% |
| TL0990 | E019D-A068S-T131S-F276W | ND | 100% |

ND—Not determined.

The stability of mannanase variants compared to parent PspMan138 was also tested in an aqueous solution of Chinese National Standard (CNS) detergent including 660 ppm PREFERENZ® P300 protease, using the following stress conditions; Stress Condition D: 85% (v/v) detergent, 37 °C incubation for 48 hours; Stress Condition E: 50% (v/v) detergent, 50 °C. incubation for 24 hours; Stress Condition F: 50% (v/v) detergent, 55 °C incubation for 48 hours. The stability results under these conditions are reported as % residual activity using the calculation described in Example 1, and the values are reported in Table 6.

TABLE 6
Stability of PspMan138 Variants in presence of protease in Chinese
National Standard detergent under stress conditions D, E, and F.

| | | % Residual Activity | | |
|---|---|---|---|---|
| | Mutations relative | Condition | Condition | Condition |
| Sample ID | to PspMan138 | D | E | F |
| PspMan138 | None | 10% | 0% | 0% |
| TL0145 | G259D | 30% | ND | ND |
| TL0148 | R261E | 40% | ND | ND |
| TL0159 | F276W | 60% | 10% | ND |
| TL0273 | G259D-F276W | 60% | 40% | ND |
| TL0274 | R261E-F276W | 60% | ND | ND |
| TL0275 | R261D-F276W | 50% | ND | ND |
| TL0276 | G259D-R261E-F276W | 40% | ND | ND |
| TL0317 | Y061W-R261E | 40% | ND | ND |
| TL0319 | Y061W-G259D-R261E-F276W | 60% | ND | ND |
| TL0320 | Y061W-T062E-G259D-R261E-F276W | 60% | ND | ND |
| TL0321 | Y061W-T062E-R261E | 40% | ND | ND |
| TL0324 | G259Q-R261E-F276W | 60% | ND | ND |
| TL0331 | Y061W-V228T-G259D-R261E-F276W | 60% | ND | ND |
| TL0332 | V228T-G259D-R261E-F276W | 60% | ND | ND |
| TL0337 | F032Y-G259D-R261E-F276W | 50% | ND | ND |
| TL0338 | F032Y-Y061W-Y167F-P168S-G259D- | 70% | ND | ND |
| | R261E-F276W | | | |
| TL0339 | F032Y-Y061W-G259D-R261E-F276W | 60% | ND | ND |
| TL0340 | F032Y-Y061W-T062E-G259D-R261E- | 60% | ND | ND |
| | F276W | | | |
| TL0373 | T062E-F276W | 60% | ND | ND |
| TL0374 | T062E-G259D | 40% | ND | ND |
| TL0376 | T062E-R261D-F276W | 70% | ND | ND |
| TL0377 | T062E-G259D-F276W | 60% | ND | ND |
| TL0381 | F032Y-R261D-F276W | 70% | ND | ND |
| TL0382 | F032Y-G259D-F276W | 70% | ND | ND |
| TL0385 | F032Y-G259D | 40% | ND | ND |
| TL0391 | F032Y-T062E-R261D-F276W | 60% | ND | ND |
| TL0392 | F032Y-T062E-G259D | 60% | ND | ND |
| TL0393 | F032Y-T062E-G259D-F276W | 70% | ND | ND |
| TL0605 | T062E-G259D-R261E-F276W | 70% | ND | ND |
| TL0606 | V059S-Q060L-G259D-R261E-F276W | 60% | ND | ND |
| TL0607 | V059S-Q060L-Y061W-G259D-R261E- | 70% | ND | ND |
| | F276W | | | |
| TL0612 | T062E-V228T-G259D-R261E-F276W | 60% | ND | ND |
| TL0613 | F032Y-T062E-G259D-R261E-F276W | 60% | ND | ND |
| TL0614 | F032Y-T062E-G259D-R261E-F276W- | 90% | 60% | ND |
| | T284E | | | |
| TL0957 | T131S-F276W | 80% | ND | ND |
| TL0987 | E019D-F276W | 90% | ND | ND |
| TL0988 | E019D-A068S-F276W | 80% | ND | ND |
| TL0989 | E019D-T131S-F276W | 100% | ND | ND |
| TL0990 | E019D-A068S-T131S-F276W | 100% | ND | ND |
| TL1047 | A161G-F276W | 80% | ND | ND |
| TL1073 | C225N-F276W | 100% | ND | ND |
| TL1074 | C225P-F276W | 100% | ND | ND |
| TL1075 | C225Q-F276W | 80% | ND | ND |
| TL1082 | F032Y-T062E-G259D-F276W-T284E | ND | 70% | ND |
| TL1083 | F032Y-T062E-V228T-G259D-F276W- | ND | 70% | ND |
| | T284E | | | |
| TL1085 | F032Y-T062E-I072V-G259D-F276W- | ND | 70% | ND |
| | T284E | | | |
| TL1086 | F032Y-T062E-A068S-G259D-F276W- | ND | 70% | ND |
| | T284E | | | |
| TL1087 | F032Y-Y061W-T062E-G259D-F276W- | ND | 70% | ND |
| | T284E | | | |
| TL1088 | F032Y-V059S-Q060L-T062E-G259D- | ND | 70% | ND |
| | F276W-T284E | | | |
| TL1090 | N010T-F032Y-T062E-G259D-F276W- | ND | 70% | ND |
| | T284E | | | |
| TL1091 | F032Y-T062E-Y167F-P168S-V228T- | ND | 80% | 50% |
| | G259D-F276W-T284E | | | |
| TL1092 | F032Y-T062E-I072V-V228T-G259D- | ND | 70% | ND |
| | F276W-T284E | | | |
| TL1093 | F032Y-T062E-I072V-Y167F-P168S- | | 80% | ND |
| | G259D-F276W-T284E | | | |
| TL1094 | F032Y-T062E-A068S-V228T-G259D- | ND | 70% | ND |
| | F276W-T284E | | | |
| TL1097 | F032Y-Y061W-T062E-V228T-G259D- | ND | 60% | ND |
| | F276W-T284E | | | |
| TL1098 | F032Y-Y061W-T062E-Y167F-P168S- | ND | 70% | ND |
| | G259D-F276W-T284E | | | |
| TL1099 | F032Y-Y061W-T062E-I072V-G259D- | ND | 80% | ND |
| | F276W-T284E | | | |
| TL1100 | F032Y-Y061W-T062E-A068S-G259D- | ND | 70% | ND |
| | F276W-T284E | | | |
| TL1101 | F032Y-V059S-Q060L-T062E-V228T- | ND | 70% | ND |
| | G259D-F276W-T284E | | | |
| TL1102 | F032Y-V059S-Q060L-T062E-Y167F- | ND | 80% | ND |
| | P168S-G259D-F276W-T284E | | | |
| TL1103 | F032Y-V059S-Q060L-T062E-I072V- | ND | 70% | ND |
| | G259D-F276W-T284E | | | |
| TL1104 | F032Y-V059S-Q060L-T062E-A068S- | ND | 70% | ND |
| | G259D-F276W-T284E | | | |
| TL1105 | F032Y-V059S-Q060L-Y061W-T062E- | ND | 70% | ND |
| | G259D-F276W-T284E | | | |
| TL1114 | N010T-F032Y-T062E-I072V-G259D- | ND | 70% | ND |
| | F276W-T284E | | | |
| TL1115 | N010T-F032Y-T062E-A068S-G259D- | ND | 70% | ND |
| | F276W-T284E | | | |
| TL1116 | N010T-F032Y-Y061W-T062E-G259D- | ND | 70% | ND |
| | F276W-T284E | | | |
| TL1117 | N010T-F032Y-V059S-Q060L-T062E- | ND | 70% | ND |
| | G259D-F276W-T284E | | | |
| TL1119 | F032Y-T062E-I072V-Y167F-P168S- | ND | 80% | ND |
| | V228T-G259D-F276W-T284E | | | |
| TL1120 | F032Y-T062E-A068S-Y167F-P168S- | ND | 80% | ND |
| | V228T-G259D-F276W-T284E | | | |
| TL1122 | F032Y-T062E-A068S-I072V-Y167F- | ND | 90% | ND |
| | P168S-G259D-F276W-T284E | | | |
| TL1123 | F032Y-Y061W-T062E-Y167F-P168S- | ND | 80% | 60% |
| | V228T-G259D-F276W-T284E | | | |
| TL1124 | F032Y-Y061W-T062E-I072V-V228T- | ND | 70% | ND |
| | G259D-F276W-T284E | | | |
| TL1125 | F032Y-Y061W-T062E-I072V-Y167F- | ND | 90% | ND |
| | P168S-G259D-F276W-T284E | | | |
| TL1126 | F032Y-Y061W-T062E-A068S-V228T- | ND | 70% | ND |
| | G259D-F276W-T284E | | | |
| TL1127 | F032Y-Y061W-T062E-A068S-Y167F- | ND | 80% | ND |
| | P168S-G259D-F276W-T284E | | | |
| TL1128 | F032Y-Y061W-T062E-A068S-I072V- | ND | 70% | ND |
| | G259D-F276W-T284E | | | |
| TL1129 | F032Y-V059S-Q060L-T062E-Y167F- | ND | 80% | ND |
| | P168S-V228T-G259D-F276W-T284E | | | |
| TL1130 | F032Y-V059S-Q060L-T062E-I072V- | ND | 70% | ND |
| | V228T-G259D-F276W-T284E | | | |
| TL1131 | F032Y-V059S-Q060L-T062E-I072V- | ND | 90% | ND |
| | Y167F-P168S-G259D-F276W-T284E | | | |
| TL1132 | F032Y-V059S-Q060L-T062E-A068S- | ND | 70% | ND |
| | V228T-G259D-F276W-T284E | | | |
| TL1133 | F032Y-V059S-Q060L-T062E-A068S- | ND | 80% | ND |
| | Y167F-P168S-G259D-F276W-T284E | | | |
| TL1134 | F032Y-V059S-Q060L-T062E-A068S- | ND | 70% | ND |
| | I072V-G259D-F276W-T284E | | | |
| TL1135 | F032Y-V059S-Q060L-Y061W-T062E- | ND | 70% | ND |
| | V228T-G259D-F276W-T284E | | | |
| TL1136 | F032Y-V059S-Q060L-Y061W-T062E- | ND | 80% | ND |
| | Y167F-P168S-G259D-F276W-T284E | | | |
| TL1137 | F032Y-V059S-Q060L-Y061W-T062E- | ND | 70% | ND |
| | I072V-G259D-F276W-T284E | | | |
| TL1138 | F032Y-V059S-Q060L-Y061W-T062E- | ND | 70% | ND |
| | A068S-G259D-F276W-T284E | | | |
| TL1155 | N010T-F032Y-T062E-I072V-V228T- | ND | 70% | ND |
| | G259D-F276W-T284E | | | |
| TL1157 | N010T-F032Y-T062E-A068S-V228T- | ND | 60% | ND |
| | G259D-F276W-T284E | | | |
| TL1159 | N010T-F032Y-T062E-A068S-I072V- | ND | 70% | ND |
| | G259D-F276W-T284E | | | |
| TL1160 | N010T-F032Y-Y061W-T062E-V228T- | ND | 60% | ND |
| | G259D-F276W-T284E | | | |
| TL1161 | N010T-F032Y-Y061W-T062E-Y167F- | ND | 70% | ND |
| | P168S-G259D-F276W-T284E | | | |
| TL1162 | N010T-F032Y-Y061W-T062E-I072V- | ND | 70% | ND |
| | G259D-F276W-T284E | | | |
| TL1165 | N010T-F032Y-V059S-Q060L-T062E- | ND | 70% | ND |
| | Y167F-P168S-G259D-F276W-T284E | | | |
| TL1166 | N010T-F032Y-V059S-Q060L-T062E- | ND | 60% | ND |
| | I072V-G259D-F276W-T284E | | | |
| TL1175 | F032Y-T062E-A068S-I072V-Y167F- | ND | 70% | ND |
| | P168S-V228T-G259D-F276W-T284E | | | |
| TL1176 | F032Y-Y061W-T062E-I072V-Y167F- | ND | 70% | ND |
| | P168S-V228T-G259D-F276W-T284E | | | |
| TL1177 | F032Y-Y061W-T062E-A068S-Y167F- | ND | 80% | 60% |
| | P168S-V228T-G259D-F276W-T284E | | | |
| TL1178 | F032Y-Y061W-T062E-A068S-I072V- | ND | 70% | ND |
| | V228T-G259D-F276W-T284E | | | |
| TL1179 | F032Y-Y061W-T062E-A068S-I072V- | ND | 80% | ND |
| | Y167F-P168S-G259D-F276W-T284E | | | |
| TL1180 | F032Y-V059S-Q060L-T062E-I072V- | ND | 70% | ND |
| | Y167F-P168S-V228T-G259D-F276W- | | | |
| | T284E | | | |
| TL1181 | F032Y-V059S-Q060L-T062E-A068S- | ND | 70% | ND |
| | Y167F-P168S-V228T-G259D-F276W- | | | |
| | T284E | | | |
| TL1182 | F032Y-V059S-Q060L-T062E-A068S- | ND | 60% | ND |
| | I072V-V228T-G259D-F276W-T284E | | | |
| TL1183 | F032Y-V059S-Q060L-T062E-A068S- | ND | 80% | ND |
| | I072V-Y167F-P168S-G259D-F276W- | | | |
| | T284E | | | |
| TL1184 | F032Y-V059S-Q060L-Y061W-T062E- | ND | 70% | ND |
| | Y167F-P168S-V228T-G259D-F276W- | | | |
| | T284E | | | |
| TL1185 | F032Y-V059S-Q060L-Y061W-T062E- | ND | 60% | ND |
| | I072V-V228T-G259D-F276W-T284E | | | |
| TL1186 | F032Y-V059S-Q060L-Y061W-T062E- | ND | 80% | ND |
| | I072V-Y167F-P168S-G259D-F276W- | | | |
| | T284E | | | |
| TL1187 | F032Y-V059S-Q060L-Y061W-T062E- | ND | 60% | ND |
| | A068S-V228T-G259D-F276W-T284E | | | |
| TL1188 | F032Y-V059S-Q060L-Y061W-T062E- | ND | 70% | ND |
| | A068S-Y167F-P168S-G259D-F276W- | | | |
| | T284E | | | |
| TL1189 | F032Y-V059S-Q060L-Y061W-T062E- | ND | 60% | ND |
| | A068S-I072V-G259D-F276W-T284E | | | |
| TL1210 | N010T-F032Y-T062E-I072V-Y167F- | ND | 70% | ND |
| | P168S-V228T-G259D-F276W-T284E | | | |
| TL1211 | N010T-F032Y-T062E-A068S-Y167F- | ND | 70% | ND |
| | P168S-V228T-G259D-F276W-T284E | | | |
| TL1212 | N010T-F032Y-T062E-A068S-I072V- | ND | 70% | ND |
| | V228T-G259D-F276W-T284E | | | |
| TL1213 | N010T-F032Y-T062E-A068S-I072V- | ND | 80% | ND |
| | Y167F-P168S-G259D-F276W-T284E | | | |
| TL1214 | N010T-F032Y-Y061W-T062E-Y167F- | ND | 70% | 50% |
| | P168S-V228T-G259D-F276W-T284E | | | |
| TL1215 | N010T-F032Y-Y061W-T062E-I072V- | ND | 60% | ND |
| | V228T-G259D-F276W-T284E | | | |
| TL1216 | N010T-F032Y-Y061W-T062E-I072V- | ND | 70% | ND |
| | Y167F-P168S-G259D-F276W-T284E | | | |
| TL1218 | N010T-F032Y-Y061W-T062E-A068S- | ND | 70% | 70% |
| | Y167F-P168S-G259D-F276W-T284E | | | |
| TL1219 | N010T-F032Y-Y061W-T062E-A068S- | ND | 70% | 50% |
| | I072V-G259D-F276W-T284E | | | |
| TL1220 | N010T-F032Y-V059S-Q060L-T062E- | ND | 70% | ND |
| | Y167F-P168S-V228T-G259D-F276W- | | | |
| | T284E | | | |
| TL1221 | N010T-F032Y-V059S-Q060L-T062E- | ND | 60% | ND |
| | I072V-V228T-G259D-F276W-T284E | | | |
| TL1222 | N010T-F032Y-V059S-Q060L-T062E- | ND | 70% | ND |
| | I072V-Y167F-P168S-G259D-F276W- | | | |
| | T284E | | | |
| TL1223 | N010T-F032Y-V059S-Q060L-T062E- | ND | 60% | ND |
| | A068S-V228T-G259D-F276W-T284E | | | |
| TL1225 | N010T-F032Y-V059S-Q060L-T062E- | ND | 70% | ND |
| | A068S-I072V-G259D-F276W-T284E | | | |
| TL1226 | N010T-F032Y-V059S-Q060L-Y061W- | ND | 60% | ND |
| | T062E-V228T-G259D-F276W-T284E | | | |
| TL1227 | N010T-F032Y-V059S-Q060L-Y061W- | ND | 70% | ND |
| | T062E-Y167F-P168S-G259D-F276W- | | | |
| | T284E | | | |
| TL1229 | N010T-F032Y-V059S-Q060L-Y061W- | ND | 60% | ND |
| | T062E-A068S-G259D-F276W-T284E | | | |
| TL1245 | F032Y-Y061W-T062E-A068S-I072V- | ND | 80% | ND |
| | Y167F-P168S-V228T-G259D-F276W- | | | |
| | T284E | | | |
| TL1246 | F032Y-V059S-Q060L-T062E-A068S- | ND | 80% | ND |
| | I072V-Y167F-P168S-V228T-G259D- | | | |
| | F276W-T284E | | | |
| TL1247 | F032Y-V059S-Q060L-Y061W-T062E- | ND | 70% | 40% |
| | I072V-Y167F-P168S-V228T-G259D- | | | |
| | F276W-T284E | | | |
| TL1248 | F032Y-V059S-Q060L-Y061W-T062E- | ND | 70% | ND |
| | A068S-Y167F-P168S-V228T-G259D- | | | |
| | F276W-T284E | | | |
| TL1249 | F032Y-V059S-Q060L-Y061W-T062E- | ND | 60% | ND |
| | A068S-I072V-V228T-G259D-F276W- | | | |
| | T284E | | | |
| TL1250 | F032Y-V059S-Q060L-Y061W-T062E- | ND | 70% | ND |
| | A068S-I072V-Y167F-P168S-G259D- | | | |
| | F276W-T284E | | | |
| TL1266 | N010T-F032Y-T062E-A068S-I072V- | ND | 70% | ND |
| | Y167F-P168S-V228T-G259D-F276W- | | | |
| | T284E | | | |
| TL1267 | N010T-F032Y-Y061W-T062E-I072V- | ND | 70% | ND |
| | Y167F-P168S-V228T-G259D-F276W- | | | |
| | T284E | | | |
| TL1268 | N010T-F032Y-Y061W-T062E-A068S- | ND | 70% | ND |
| | Y167F-P168S-V228T-G259D-F276W- | | | |
| | T284E | | | |
| TL1269 | N010T-F032Y-Y061W-T062E-A068S- | ND | 60% | ND |
| | I072V-V228T-G259D-F276W-T284E | | | |
| TL1271 | N010T-F032Y-V059S-Q060L-T062E- | ND | 70% | ND |
| | I072V-Y167F-P168S-V228T-G259D- | | | |
| | F276W-T284E | | | |
| TL1272 | N010T-F032Y-V059S-Q060L-T062E- | ND | 70% | 50% |
| | A068S-Y167F-P168S-V228T-G259D- | | | |
| | F276W-T284E | | | |
| TL1273 | N010T-F032Y-V059S-Q060L-T062E- | ND | 70% | ND |
| | A068S-I072V-V228T-G259D-F276W- | | | |
| | T284E | | | |
| TL1274 | N010T-F032Y-V059S-Q060L-T062E- | ND | 80% | ND |
| | A068S-I072V-Y167F-P168S-G259D- | | | |
| | F276W-T284E | | | |
| TL1275 | N010T-F032Y-V059S-Q060L-Y061W- | ND | 70% | ND |
| | T062E-Y167F-P168S-V228T-G259D- | | | |
| | F276W-T284E | | | |
| TL1277 | N010T-F032Y-V059S-Q060L-Y061W- | ND | 70% | ND |
| | T062E-I072V-Y167F-P168S-G259D- | | | |
| | F276W-T284E | | | |
| TL1279 | N010T-F032Y-V059S-Q060L-Y061W- | ND | 70% | ND |
| | T062E-A068S-Y167F-P168S-G259D- | | | |
| | F276W-T284E | | | |
| TL1280 | N010T-F032Y-V059S-Q060L-Y061W- | ND | 70% | ND |
| | T062E-A068S-I072V-G259D-F276W- | | | |
| | T284E | | | |
| TL1308 | N010T-F032Y-Y061W-T062E-A068S- | ND | 80% | 70% |
| | I072V-Y167F-P168S-V228T-G259D- | | | |
| | F276W-T284E | | | |
| TL1309 | N010T-F032Y-V059S-Q060L-T062E- | ND | 80% | ND |
| | A068S-I072V-Y167F-P168S-V228T- | | | |
| | G259D-F276W-T284E | | | |
| TL1310 | N010T-F032Y-V059S-Q060L-Y061W- | ND | 70% | 40% |
| | T062E-I072V-Y167F-P168S-V228T- | | | |
| | G259D-F276W-T284E | | | |
| TL1311 | N010T-F032Y-V059S-Q060L-Y061W- | ND | 80% | ND |
| | T062E-A068S-Y167F-P168S-V228T- | | | |
| | G259D-F276W-T284E | | | |
| TL1312 | N010T-F032Y-V059S-Q060L-Y061W- | ND | 70% | ND |
| | T062E-A068S-I072V-V228T-G259D- | | | |
| | F276W-T284E | | | |
| TL1313 | N010T-F032Y-V059S-Q060L-Y061W- | ND | 80% | ND |
| | T062E-A068S-I072V-Y167F-P168S- | | | |
| | G259D-F276W-T284E | | | |
| TL1330 | N010T-F032Y-V059S-Q060L-Y061W- | ND | 80% | ND |
| | T062E-A068S-I072V-Y167F-P168S- | | | |
| | V228T-G259D-F276W-T284E | | | |
| TL1432 | P168A-G259D-F276W | ND | 70% | ND |
| TL1433 | P168T-G259D-F276W | ND | 70% | ND |
| TL1601 | F032Y-Y061W-T062E-G259D-R261E- | ND | 70% | ND |
| | F276W-T284E | | | |
| TL1603 | F032Y-T062E-A068S-G259D-R261E- | ND | 70% | ND |
| | F276W-T284E | | | |
| TL1614 | F032Y-T062E-T131S-G259D-R261E- | ND | 70% | ND |
| | F276W-T284E | | | |
| TL1617 | F032Y-T062E-N150D-G259D-R261E- | ND | 70% | ND |
| | F276W-T284E | | | |
| TL1623 | F032Y-T062E-P168S-G259D-R261E- | ND | 70% | ND |
| | F276W-T284E | | | |
| TL1629 | F032Y-T062E-L235K-G259D-R261E- | ND | 70% | ND |
| | F276W-T284E | | | |
| TL1656 | G259E-F276W | ND | 40% | ND |
| TL1716 | N010T-F276W | ND | 20% | ND |
| TL1743 | K093Q-F276W | ND | 20% | ND |
| TL1749 | D139R-F276W | ND | 20% | ND |
| TL1750 | A136P-F276W | ND | 30% | ND |
| TL1795 | N010T-F032Y-Y061W-T062E-P168A- | ND | ND | 80% |
| | V228T-G259D-F276W-T284E | | | |
| TL1796 | N010T-F032Y-Y061W-T062E-Y167F- | ND | ND | 60% |
| | P168S-V228T-G259E-F276W-T284E | | | |
| TL1797 | F032Y-Y061W-T062E-T131S-Y167F- | ND | ND | 70% |
| | P168S-V228T-G259D-F276W-T284E | | | |
| TL1800 | N010T-F032Y-Y061W-T062E-A136P- | ND | ND | 70% |
| | Y167F-P168S-V228T-G259D-F276W- | | | |
| | T284E | | | |
| TL1805 | N010T-F032Y-Y061W-T062E-K093Q- | ND | ND | 60% |
| | Y167F-P168S-V228T-G259D-F276W- | | | |
| | T284E | | | |
| TL1806 | N010T-F032Y-Y061W-T062E-Y167F- | ND | ND | 60% |
| | P168S-V228T-L235K-G259D-F276W- | | | |
| | T284E | | | |
| TL1807 | N010T-F032Y-Y061W-T062E-D139R- | ND | ND | 70% |
| | Y167F-P168S-V228T-G259D-F276W- | | | |
| | T284E | | | |
| TL1812 | N010T-F032Y-Y061W-T062E-N150D- | ND | ND | 60% |
| | Y167F-P168S-V228T-G259D-F276W- | | | |
| | T284E | | | |
| TL1884 | N010T-F032Y-Y061W-T062E-Y167F- | ND | ND | 50% |
| | P168A-V228T-G259D-F276W-T284E | | | |
| TL1885 | N010T-F032Y-Y061W-T062E-T131S- | ND | ND | 50% |
| | Y167F-P168S-V228T-G259D-F276W- | | | |
| | T284E | | | |
| TL1898 | N010T-F032Y-Y061W-T062E-P168S- | ND | ND | 50% |
| | V228T-G259D-F276W-T284E | | | |
| TL1903 | N010T-F032Y-Y061W-T062E-A068S- | ND | ND | 60% |
| | Y167F-P168A-G259D-F276W-T284E | | | |
| TL1906 | N010T-F032Y-Y061W-T062E-A068S- | ND | ND | 70% |
| | Y167F-P168A-G259E-F276W-T284E | | | |

ND—Not determined.

What is claimed is:

1. A mannanase variant comprising an amino acid substitution at a position selected from the group consisting of 32, 72, 161 and 172, wherein the amino acid positions of the variant are numbered by correspondence with the amino acid sequence of SEQ ID NO: 1, and wherein the variant has at least 80% identity to the amino acid sequence of SEQ ID NO: 1.

2. A mannanase variant comprising an amino acid substitution, wherein said variant comprises an amino acid substitution selected from the group consisting of 19D, 32Y, 34D, 72V, 93Q, 131S, 136P, 139R, 161G, 172F, 225N/Q, 259D, 261D/E and 276W, wherein the amino acid positions of the variant are numbered by correspondence with the amino acid sequence of SEQ ID NO: 1, and wherein the variant has at least 80% identity to the amino acid sequence of SEQ ID NO: 1.

3. A mannanase variant comprising two or more amino acid substitutions selected from the group consisting of 19D, 32Y, 72V, 93Q, 131S, 136P, 139R, 161G, 168T, 172F, 225N/Q, 259D/E, 261D/E and 276W, wherein the amino acid positions of the variant are numbered by correspondence with the amino acid sequence of SEQ ID NO: 1, and wherein the variant has at least 80% identity to the amino acid sequence of SEQ ID NO: 1.

4. The mannanase variant of claim 3, wherein the variant comprises amino acid substitutions selected from the group consisting of 19D-276W, 32Y-259D, 93Q-276W, 131S-276W, 136P-276W, 139R-276W, 161G-276W, 225N/Q-276W, 259D/E-276W, and 261D/E-276W.

5. The mannanase variant of claim 3, wherein the variant comprises amino acid substitutions selected from the group consisting of 19D-131S-276W, 32Y-261D-276W, 32Y-259D-276W, 32Y-172F-259D, 168T-259D-276W, 259D-261E-276W, and 259Q-261E-276W.

6. A mannanase variant comprising amino acid substitutions selected from the group consisting of Y061W-G259D-R261E-F276W, Y061W-T062E-G259D-R261E-F276W, Y061W-V228T-G259D-R261E-F276W, V228T-G259D-R261E-F276W, F032Y-G259D-R261E-F276W, F032Y-Y061W-Y167F-P168S-G259D-R261E-F276W, F032Y-Y061W-G259D-R261E-F276W, F032Y-Y061W-T062E-G259D-R261E-F276W, F032Y-T062E-R261D-F276W, F032Y-T062E-G259D-F276W, T062E-G259D-R261E-F276W, V059S-Q060L-G259D-R261E-F276W, V059S-Q060L-Y061W-G259D-R261E-F276W, V059S-Q060L-T062E-G259D-R261E-F276W, T062E-V228T-G259D-R261E-F276W, F032Y-T062E-G259D-R261E-F276W, F032Y-T062E-G259D-R261E-F276W-T284E, E019D-A068S-T131S-F276W, F032Y-T062E-G259D-F276W-T284E, F032Y-T062E-V228T-G259D-F276W-T284E, F032Y-T062E-I072V-G259D-F276W-T284E, F032Y-T062E-A068S-G259D-F276W-T284E, F032Y-Y061W-T062E-G259D-F276W-T284E, F032Y-V059S-Q060L-T062E-G259D-F276W-T284E, N010T-F032Y-T062E-G259D-F276W-T284E, F032Y-T062E-Y167F-P168S-V228T-G259D-F276W-T284E, F032Y-T062E-I072V-V228T-G259D-F276W-T284E, F032Y-T062E-I072V-Y167F-P168S-G259D-F276W-T284E, F032Y-T062E-A068S-V228T-G259D-F276W-T284E, F032Y-Y061W-T062E-V228T-G259D-F276W-T284E, F032Y-Y061W-T062E-Y167F-P168S-G259D-F276W-T284E, F032Y-Y061W-T062E-1072V-G259D-F276W-T284E, F032Y-Y061W-T062E-A068S-G259D-F276W-T284E, F032Y-V059S-Q060L-T062E-V228T-G259D-F276W-T284E, F032Y-V059S, Q060L-T062E-Y167F-P168S-G259D-F276W-T284E, F032Y-V059S-Q060L-T062E-I072V-G259D-F276W-T284E, F032Y-V059S-Q060L-T062E-A068S-G259D-F276W-T284E, F032Y-V059S-Q060L-Y061W-T062E-G259D-F276W-T284E, N010T-F032Y-T062E-I072V-G259D-F276W-T284E, N010T-F032Y-T062E-A068S-G259D-F276W-T284E, N010T-F032Y-Y061W-T062E-G259D-F276W-T284E, N010T-F032Y-V059S-Q060L-T062E-G259D-F276W-T284E, F032Y-T062E-I072V-Y167F-P168S-V228T-G259D-F276W-T284E, F032Y-T062E-A068S-Y167F-P168S-V228T-G259D-F276W-T284E, F032Y-T062E-A068S-I072V-Y167F-P168S-G259D-F276W-T284E, F032Y-Y061W-T062E-Y167F-P168S-V228T-G259D-F276W-T284E, F032Y-Y061W-T062E-I072V-V228T-G259D-F276W-T284E, F032Y-Y061W-T062E-1072V-Y167F-P168S-G259D-F276W-T284E, F032Y-Y061W-T062E-A068S-V228T-G259D-F276W-T284E, F032Y-Y061W-T062E-A068S-Y167F-P168S-G259D-F276W-T284E, F032Y-Y061W-T062E-A068S-I072V-G259D-F276W-T284E, F032Y-V059S-Q060L-T062E-Y167F-P168S-V228T-G259D-F276W-T284E, F032Y-V059S-Q060L-T062E-I072V-V228T-G259D-F276W-T284E, F032Y-V059S-Q060L-T062E-1072V-Y167F-P168S-G259D-F276W-T284E, F032Y-V059S-Q060L-T062E-A068S-V228T-G259D-F276W-T284E, F032Y-V059S-Q060L-T062E-A068S-Y167F-P168S-G259D-F276W-T284E, F032Y-V059S-Q060L-T062E-A068S-1072V-G259D-F276W-T284E, F032Y-V059S-Q060L-Y061W-T062E-V228T-G259D-F276W-T284E, F032Y-V059S-Q060L-Y061W-T062E-Y167F-P168S-G259D-F276W-T284E, F032Y-V059S-Q060L-Y061W-T062E-A068S-G259D-F276W-T284E, N010T-F032Y-T062E-I072V-V228T-G259D-F276W-T284E, N010T-F032Y-T062E-A068S-V228T-G259D-F276W-T284E, N010T-F032Y-T062E-A068S-I072V-G259D-F276W-T284E, N010T-F032Y-Y061W-T062E-V228T-G259D-F276W-T284E, N010T-F032Y-Y061W-T062E-Y167F-P168S-G259D-F276W-T284E, N010T-F032Y-Y061W-T062E-I072V-G259D-F276W-T284E, N010T-F032Y-V059S-Q060L-T062E-Y167F-P168S-G259D-F276W-T284E, N010T-F032Y-V059S-Q060L-T062E-I072V-G259D-F276W-T284E, F032Y-T062E-A068S-1072V-Y167F-P168S-V228T-G259D-F276W-T284E, F032Y-Y061W-T062E-1072V-Y167F-P168S-V228T-G259D-F276W-T284E, F032Y-Y061W-T062E-A068S-Y167F-P168S-V228T-G259D-F276W-T284E, F032Y-Y061W-T062E-A068S-I072V-V228T-G259D-F276W-T284E, F032Y-Y061W-T062E-A068S-I072V-Y167F-P168S-G259D-F276W-T284E, F032Y-V059S-Q060L-T062E-I072V-Y167F-P168S-V228T-G259D-F276W-T284E, F032Y-V059S-Q060L-T062E-A068S-Y167F-P168S-V228T-G259D-F276W-T284E, F032Y-V059S-Q060L-T062E-A068S-1072V-V228T-G259D-F276W-T284E, F032Y-V059S-Q060L-T062E-A068S-I072V-Y167F-P168S-G259D-F276W-T284E, F032Y-V059S-Q060L-Y061W-T062E-Y167F-P168S-V228T-G259D-F276W-T284E, F032Y-V059S-Q060L-Y061W-T062E-I072V-V228T-G259D-F276W-T284E, F032Y-V059S-Q060L-Y061W-T062E-I072V-Y167F-P168S-G259D-F276W-T284E, F032Y-V059S-Q060L-Y061W-T062E-A068S-V228T-G259D-F276W-T284E, F032Y-V059S-Q060L-Y061W-T062E-A068S-Y167F-P168S-G259D-F276W-T284E, F032Y-V059S-Q060L-Y061W-T062E-A068S-I072V-G259D-F276W-T284E, N010T-F032Y-T062E-I072V-Y167F-P168S-V228T-G259D-F276W-T284E, N010T-F032Y-T062E-A068S-Y167F-P168S-V228T-G259D-F276W-T284E, N010T-F032Y-T062E-A068S-I072V-V228T-G259D-F276W-T284E, N010T-F032Y-T062E-A068S-I072V-Y167F-P168S-G259D-F276W-T284E, N010T-F032Y-Y061W-T062E-Y167F-P168S-V228T-G259D-F276W-T284E, N010T-F032Y-Y061W-T062E-I072V-V228T-G259D-F276W-T284E, N010T-F032Y-Y061W-T062E-I072V-Y167F-P168S-G259D-F276W-T284E, N010T-F032Y-Y061W-T062E-A068S-Y167F-P168S-G259D-F276W-T284E, N010T-F032Y-Y061W-T062E-A068S-I072V-G259D-F276W-T284E, N010T-F032Y-V059S-Q060L-T062E-1072V-V228T-G259D-F276W-T284E, N010T-F032Y-V059S-Q060L-T062E-1072V-Y167F-P168S-G259D-F276W-T284E, N010T-F032Y-V059S-Q060L-T062E-A068S-V228T-G259D-F276W-T284E, N010T-F032Y-V059S-Q060L-T062E-A068S-1072V-G259D-F276W-T284E, N010T-F032Y-V059S-Q060L-Y061W-T062E-V228T-G259D-F276W-T284E, N010T-F032Y-V059S-Q060L-Y061W-T062E-Y167F-P168S-G259D-F276W-T284E, N010T-F032Y-V059S-Q060L-Y061W-T062E-A068S-G259D-F276W-T284E, F032Y-Y061W-T062E-A068S-I072V-Y167F-P168S-V228T-G259D-F276W-T284E, F032Y-V059S-Q060L-T062E-A068S-I072V-Y167F-P168S-V228T-G259D-F276W-T284E, F032Y-V059S-Q060L-Y061W-T062E-I072V-Y167F-P168S-V228T-G259D-F276W-T284E, F032Y-V059S-Q060L-Y061W-T062E-A068S-Y167F-P168S-V228T-G259D-F276W-T284E, F032Y-V059S-Q060L-Y061W-T062E-A068S-1072V-V228T-G259D-F276W-T284E, F032Y-V059S-Q060L-Y061W-T062E-A068S-1072V-Y167F-P168S-G259D-F276W-T284E, N010T-F032Y-T062E-A068S-I072V-Y167F-P168S-V228T-G259D-F276W-T284E, N010T-F032Y-Y061W-T062E-I072V-Y167F-P168S-V228T-G259D-F276W-T284E, N010T-F032Y-Y061W-T062E-A068S-Y167F-P168S-V228T-G259D-F276W-T284E, N010T-F032Y-Y061W-T062E-A068S-I072V-V228T-G259D-F276W-T284E, N010T-F032Y-V059S-Q060L-T062E-1072V-Y167F-P168S-V228T-G259D-F276W-T284E, N010T-F032Y-V059S-Q060L-T062E-A068S-Y167F-P168S-V228T-G259D-F276W-T284E, N010T-F032Y-V059S-Q060L-T062E-A068S-I072V-V228T-G259D-F276W-T284E, N010T-F032Y-V059S-Q060L-T062E-A068S-1072V-Y167F-P168S-G259D-F276W-T284E, N010T-F032Y-V059S-Q060L-Y061W-T062E-Y167F-P168S-V228T-G259D-F276W-T284E, N010T-F032Y-V059S-Q060L-Y061W-T062E-1072V-Y167F-P168S-G259D-F276W-T284E, N010T-F032Y-V059S-Q060L-Y061W-T062E-A068S-Y167F-P168S-G259D-F276W-T284E, N010T-F032Y-V059S-Q060L-Y061W-T062E-A068S-1072V-G259D-F276W-T284E, N010T-F032Y-Y061W-T062E-A068S-1072V-Y167F-P168S-V228T-G259D-F276W-T284E, N010T-F032Y-V059S-Q060L-T062E-A068S-I072V-Y167F-P168S-V228T-G259D-F276W-T284E, N010T-F032Y-V059S-Q060L-Y061W-T062E-I072V-Y167F-P168S-V228T-G259D-F276W-T284E, N010T-F032Y-V059S-Q060L-Y061W-T062E-A068S-Y167F-P168S-V228T-G259D-F276W-T284E, N010T-F032Y-V059S-Q060L-Y061W-T062E-A068S-I072V-V228T-G259D-F276W-T284E, N010T-F032Y-V059S-Q060L-Y061W-T062E-A068S-I072V-Y167F-P168S-G259D-F276W-T284E and N010T-F032Y-V059S-Q060L-Y061W-T062E-A068S-I072V-Y167F-P168S-V228T-G259D-F276W-T284E, F032Y-Y061W-T062E-G259D-R261E-G259D-R261E-F276W-T284E, F032Y-T062E-N150D-G259D-R261E-F276W-T284E, F032Y-T062E-P168S-G259D-R261E-F276W-T284E, F032Y-T062E-L235K-G259D-R261E-F276W-T284E, N010T-F032Y-Y061W-T062E-P168A-V228T-G259D-F276W-T284E, N010T-F032Y-Y061W-T062E-Y167F-P168S-V228T-G259E-F276W-T284E, F032Y-Y061W-T062E-T131S-Y167F-P168S-V228T-G259D-F276W-T284E, N010T-F032Y-Y061W-T062E-A136P-Y167F-P168S-V228T-G259D-F276W-T284E, N010T-F032Y-Y061W-T062E-K093Q-Y167F-P168S-V228T-G259D-F276W-T284E, N010T-F032Y-Y061W-T062E-Y167F-P168S-V228T-L235K-G259D-F276W-T284E, N010T-F032Y-Y061W-T062E-D139R-Y167F-P168S-V228T-G259D-F276W-T284E, N010T-F032Y-Y061W-T062E-N150D-Y167F-P168S-V228T-G259D-F276W-T284E, N010T-F032Y-Y061W-T062E-Y167F-P168A-V228T-G259D-F276W-T284E, N010T-F032Y-Y061W-T062E-T131S-Y167F-P168S-V228T-G259D-F276W-T284E, N010T-F032Y-Y061W-T062E-P168S-V228T-G259D-F276W-T284E, N010T-F032Y-Y061W-T062E-A068S-Y167F-P168A-G259D-F276W-T284E, and N010T-F032Y-Y061W-T062E-A068S-Y167F-P168A-G259E-F276W-T284E, wherein the amino acid positions of the variant are numbered by correspondence with the amino acid sequence of SEQ ID NO: 1, and wherein the variant has at least 80% identity to the amino acid sequence of SEQ ID NO: 1.

7. The mannanase variant of claim 1, wherein the variant comprises an amino acid sequence having at least 85%, 90%, 91%, 92%, 93%, 94%, 95%, 96%, 97%, 98%, or 99% sequence identity to the amino acid sequence of SEQ ID NO: 1.

8. The mannanase variant according to claim 1, wherein said variant is derived from a parent or reference polypeptide with 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1.

9. The mannanase variant of claim 1, wherein said variant has improved stability when compared to a parent or reference mannanase.

10. The mannanase of claim 9, wherein said variant has equal or improved cleaning performance in a detergent when compared to a parent or reference mannanase.

11. The mannanase variant of claim 1, wherein the mannanase variant has mannanase activity.

12. A polynucleotide comprising a nucleic acid sequence encoding a variant of claim 1, wherein said polynucleotide is, optionally, isolated.

13. An enzyme composition comprising one or more mannanase variant according to claim 1.

14. The enzyme composition according to claim 13, wherein said composition is an enzyme granule.

15. The enzyme composition according to claim 13, further comprising one or more other enzymes selected from acyl transferases, amylases, alpha-amylases, beta-amylases, alpha-galactosidases, arabinases, arabinosidases, aryl esterases, beta-galactosidases, beta-glucanases, carrageenases, catalases, cellulases, chondroitinases, cutinases, dispersins, endo-glucanases, endo-beta-mannanases, exo-beta-mannanases, esterases, exo-mannanases, galactanases, glucoamylases, hemicellulases, hexosaminidase, hyaluronidases, keratinases, laccases, lactases, ligninases, lipases, lipolytic enzymes, lipoxygenases, lysozyme, mannanases, metalloproteases, nucleases, oxidases, oxidoreductases, pectate lyases, pectin acetyl esterases, pectinases, pentosanases, perhydrolases, peroxidases, PETases, phenoloxidases, phosphatases, phospholipases, phytases, polyesterases, polygalacturonases, proteases, pullulanases, reductases, rhamnogalacturonases, subtilisins, tannases, transglutaminases, xylan acetyl-esterases, xylanases, and xylosidases; and combinations thereof.

16. A cleaning composition comprising the mannanase variant of claim 1.

17. The cleaning composition of claim 16, wherein the cleaning composition is a detergent composition selected from the group consisting of a laundry detergent, a fabric softening detergent, a dishwashing detergent, a medical instrument cleaning detergent, and a hard-surface cleaning detergent.

18. A method of cleaning comprising, contacting a surface or an item in need of cleaning with an effective amount of a mannanase variant of claim 1 or the enzyme composition of claim 13; and optionally further comprising the step of rinsing said surface or item after contacting said surface or item with said variant or enzyme composition.

19. The method of claim 18, wherein said item is a medical instrument, dishware or fabric.
